SEQUENCE LISTING (184 sequences)

SEQ ID NO: 1 - protein, 265 aa, Arabidopsis thaliana
MAQTMLLTSGVTAGHFLRNKSPLAQPKVHHLFLSGNSPVALPSRRQSFVPLALFKPKTKAAPKKVEKPKSKVEDGIFGTSGGIGFTKANELFVGRVAMIGFAASLLGEALTGKGILAQLNLETGIPIYEAEPLLLFFILFTLLGAIGALGDRGKFVDDPPTGLEKAVIPPGKNVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 2 - protein, 268 aa, Oryza sativa
MAQSMLVSGANGTVAAASTSRLQPVRPTPFSRLVLSQPSSSLGRAVSVKTVALFGRSKTKAAPARKAEPKPKFKTEDGIFGTSGGIGFTKENELFVGRVAMLGFAASILGEAITGKGILAQLNLETGIPIYEAEPLLLFFILFTLLGAIGALGDRGSFVDDQPVTGLDKAVIAPGKGFRSALGLSEGGPLFGFTKANELFVGRLAQLGIAFSIIGEIITGKGALAQLNIETGVPINEIEPLVLFNVVFFFIAAINPGTGKFVSDDDEE

SEQ ID NO: 3 - protein, 248 aa, Oryza sativa
MAMPMMVVSGLGTAPRSSPMVQLQRMKKHLVVVAAFKSRTKASPKVDKSNKNKSIVEDGIFGTSGGIGFTKENELFVGRVAMLGFAASLLGEAVTGKGILAQLNLETGIPIYEAEPLLLFFILFTLLGAIGALGDRGRFVDDATGLERAVIPPGKGFRAALGLSEGGPLFGFTKANELFVGRLAQLGIAFSLIGEIITGKGALAQLNIETGVPINEIEPLLLFNILFFFFAAINPGTGKFVTDDNDDQ

SEQ ID NO: 4 - protein, 248 aa, Oryza sativa
MAMPMMVVSDLGTAPRSSPMVQLQRMKKHLVVVAAFKSRTKASPKVDKSNKNKSIVEDGIFGTSGGIGFTKENELFVGRVAMLGFAASLLGEAVTGKGILAQLNLETGIPIYEAEPLLLFFILFTLLGAIGALGDRGRFVDDATGLERAVIPPGKGFRAALGLSEGGPLFGFTKANELFVGRLAQLGIAFSLIGEIITGKGALAQLNIETGVPINEIEPLLLFNILFFFFAAINPGTGKFVTDDNDDQ

SEQ ID NO: 5 - protein, 254 aa, Oryza sativa
MALQQSMAMPMMVVSDLGTAPRSSPMVQLQRMKKHLVVVAAFKSRTKASPKVDKSNKNKSIVEDGIFGTSGGIGFTKENELFVGRVAMLGFAASLLGEAVTGKGILAQLNLETGIPIYEAEPLLLFFILFTLLGAIGALGDRGRFVDDATGLERAVIPPGKGFRAALGLSEGGPLFGFTKANELFVGRLAHVGIAFSLIGEIITGKGALAQLNIETGVPINEIEPLLLFNILFFFFAAINPGTGKFVTDDNDDQ

SEQ ID NO: 6 - protein, 254 aa, Oryza sativa
MALQQSMAMPMMVVSDLGTAPRSSPMVQLQRMKKHLVVVAAFKSRTKASPKVDKSNKNKSIVEDGIFGTSGGIGFTKENELFVGRVAMLGFAASLLGEAVTGKGILAQLNLETGIPIYEAEPLLLFFILFTLLGAIGALGDRGRFVDDATGLERAVIPPGKGFRAALGLSEGGPLFGFTKANELFVGRLAQLGIAFSLIGEIITGKGALAQLNIETGVPINEIEPLLLFNILFFFFAAINPGTGKFVTDDNDDQ

SEQ ID NO: 7 - protein, 269 aa, Vigna unguiculata
MAQTMLLMSSVSSSHSLGLKKDLFLQLRPKFSQLSFNPLPSSTPFPSSPRTFTTLALFKSKTKAPPAKTKVVKPKQKVEDGIFGTSGGFGFTKQNELFVGRVAMLGFAASLLGEGVTGKGILAQLNLETGIPIYEAEPLLLFFILFTLLGAIGGLGDRGKFVDDEEPNTGGVIPGGKGFREALGLGSGPLFGFTKANELFVGRLAQLGFVFSLIGEIVTGKGALAQLNIETGVPINEIEPLVLFNVLFFFVAALNPGTGKFVTDEGDDE

SEQ ID NO: 8 - protein, 273 aa, Glycine max
MAQTMLLMSSVSSSYSVDLKKDLFLQLQSQSLRPKFSQLSFNPLPPSTSSFSSPRTFTTLALFKSKTKAAPAKTKVTKPKQKVEDGIFGTSGGFGFTKQNELFVGRVAMLGFAASLLGEGITGKGILAQLNLETGIPIYEAEPLLLFFILFTLLGAIGGLGDRGKFVDDEPTTGGVIPSGKGFREALGLGSGPLFGFTKANELFVGRLAQLGFVFSLIGEIITGKGALAQLNIETGVPINEIEPLVLFNVLFFFIAALNPGTGKFVTDDGEDD

SEQ ID NO: 9 - protein, 273 aa, Glycine max
MAQTMLLMSSVSSSYSVDLKKDLFLQLQSQSLRPKFSQLSFNPLPSSTSSFSSPRTFTTLALFKSKTKAAPAKTKVTKPKQKVEDGIFGTSGGFGFTKQNELFVGRVAMLGFAASLLGEGITGKGILAQLNLETGIPLYEAEPLLLFFILFTLLGAIGGLGDRGKFVDDEPTTGGVIPPGKGFREALGLGSGPLFGFTKANELFVGRLAQLGFVFSLIGEIITGKGALAQLNIETGVPINEIEPLVLFNVLFFFIAALNPGTGKFVTDEGEDD

SEQ ID NO: 10 - protein, 273 aa, Glycine max
MAQTMLLMSSVSSSYSVDLKKDLFLQLQSQSLRPKFSQLSFNPLPPSTSSFSSPRTFTTLALFKSKTKAAPAKTKVTKPKQKVEDGIFGTSGGFGFTKQNELFVGRVAMLGFAASLLGEGITGKGILAQLNLETGIPIYEAEPLLLFFILFTLLGAIGGLGDRGKFVDDEPTTGGVIPSGKGFREALGLGSGPLFGFTKANELFVGRLAQLGFVSSLIGEIITGKGALAQLNIETGVPINEIEPLVLFNVLFFFIAALNPGTGKFVTGDGEDD

SEQ ID NO: 11 - protein, 273 aa, Glycine max
MAQTMLLMSSVSSSYSVDLKKDLFLQLQSQSLRPKFSQLSFNPLPSSTSSFSSPRTFTTLALFKSKTKAAPAKTKVTKPKQKVEDGIFGTSGGFGFTKQNELFVGRVAMLGFAASLLGEGITGKGILAQLNLETGIPLYEAEPLLLFFILFTLLGAIGGLGDRGKFVDDEPTTGGVIPPGKGFREALGLGSGPLFGFTKANEPFVGRLAQLGFVFSLIGEIITGKGALAQLNIETGVPINEIEPLVLFNVLFFFIAALNPGTGKFVTDEGEDD

SEQ ID NO: 12 - protein, 272 aa, Zea mays
MAAQSMLMSTSVNGGRALPSLQAVRPAPYPRLPLPSSSSSSAGYRHSKSVSVKTLALFGKSKVKTAPAKKAAAPKPKPKVEDGIFGTSGGIGFTKENELFVGRVAMLGFAASLLGEAITGKGILAQLNLETGIPIYEAEPLLLFFILFTLLGAIGALGDRGRFVDEEVTGLDKAVIQPGKGFRGALGLSEGGPLFGFTKSNELFVGRMAQLGVAFSIIGEIITGKGALAQLNIETGVPINEIEPLVIFNVLFFFIAAINPGNGRFIIGEEEE

SEQ ID NO: 13 - protein, 271 aa, Zea mays
MAAQSMLMSTSVNGGRALPSLQAVRPAPYPRLPLPSSSSSSGYRHSKSVSVKTLALFGKSKVKTAPAKKAAAPKPKPKVEDGIFGTSGGIGFTKENELFVGRVAMLGFAASLLGEAITGKGILAQLNLETGIPIYEAEPLLLFFILFTLLGAIGALGDRGRFVDEEVTGLDKAVIQPGKGFRGALGLSEGGPLFGFTKSNELFVGRMAQLGVAFSIIGEIITGKGALAQLNIETGVPINEIEPLVIFNVLFFFIAAINPGNGRFIIGEEEE

SEQ ID NO: 14 - protein, 265 aa, Zea mays
MSTSVNGGRALPSLQAVRPAPYPRLPLPSSSSSSAGYRHSKSVSVKTLALFGKSKVKTAPAKKAAAPKPKPKVEDGIFGTSGGIGFTKENELFVGRVAMLGFAASLLGEAITGKGILAQLNLETGIPIYEAEPLLLFFILFTLLGAIGALGDRGRFVDEEVTGLDKAVIQPGKGFRGALGLSEGGPLFGFTKSNELFVGRMAQLGVAFSIIGEIITGKGALAQLNIETGVPINEIEPLVIFNVLFFFIAAINPGNGRFIIGEEEE

SEQ ID NO: 15 - protein, 274 aa, Nicotiana tabacum
MAQTMLLTANAKVDLRSKESLVERLKPKPLSSFFLPSLPLKYPSASSSSSSHFASTTVALFKSKAKAPAKKVVPKPKEKVEDGIFGTSGGIGFTKQNELFVGRVAMIGFAASLLGEAVTGKGILAQLNLETGIPIYEAEPLLLFFILFNLLGAIGALGDRGKFIDDPAPATGLDKAVIPPGKGFKAALGLREGGPLFGFTKANELFVGRLAQLGIAFSIIGEIITGKGALAQLNFETGVPINEIEPLLLFNIVFFFIAAINPGTGKFVTDEEEE

SEQ ID NO: 16 - protein, 274 aa, Nicotiana tabacum
MAQTMLLTANAKVDLRSKESLVERLKPKPLSSFFLPSLPLKYPSASASASSHFTSTTVALFKSKAKAPAKKVVPKPKEKVEDGIFGTSGGIGFTKQNELFVGRVAMIGFAASLLGEAITGKGILAQLNLETGIPIYEAEPLLLFFILFNLLGAIGALEDRGKFIDDPAPPTGLDKAVIPPGKGFKSALGLSEGGPLFEFTKANELFVGRLAQLGIAFSIIGEIITGKGALAQLNFETGVPINEIEPLLLFNIVFFFVAAINPGTGKFVTDEEEE

SEQ ID NO: 17 - protein, 274 aa, Nicotiana benthamiana
MAQTMLLTANAKVDLRNKEPLVERLKPKPLSSFFLPSLPLKYPSASSSSSSHFTSTTVALFKSKAKAPAKKVVPKPKEKVEDGIFGTSGGIGFTKQNELFVGRVAMIGFAASLLGEAITGKGILAQLNLETGIPIYEAEPLLLFFILFNLLGAIGALGDRGKFIDDPVPATGLDKAVIPPGKGFKSALGLSEGGPLFGFTKANELFVGRLAQLGIAFSIIGEIITGKGALAQLNFETGVPINEIEPLLLFNIVFFFVAAINPGTGKFVTDEEEE

SEQ ID NO: 18 - protein, 277 aa, Nicotiana benthamiana
MAQTMLLTANAKVDLRSKESLVERLKPKPLSSFFLPSLSLKYPSASSSSSSSSSHFTSTTVALFKSKAKAPAKKVVPKPKEKVEDGIFGTSGGIGFTKQNELFVGRVAMIGFAASLLGEAITGKGILAQLNLETGIPIYEAEPLLLFFILFNLLGAIGALGDRGKFIDDPTPPTGLDKAVIPPGKGFKSALGLSEGGPLFGFTKANELFVGRLAQLGIAFSIIGEIITGKGALAQLNFETGVPINEIEPLLLFNIVFFFVAAINPGTGKFVTDEEEE

SEQ ID NO: 19 - protein, 273 aa, Hevea brasiliensis
MAQTILFMSGVSTKHVVNLKRDPLLQFQVERLRPKPFSRLFFNPLPSNSSFSSKTFTTLALFKSKTKAPLKKAAEPKPKVEDGIFGTSGGIGFTKQNELFVGRVAMLGFAASLLGEAITGKGILAQLNLETGIPIYEAEPLLLFFILFTLLGAIGALGDRGKFVDDPPTGIEGAVIPPGKGFRSALGLKEGGPLFGFTKANELFVGRLAQLGIAFSLIGEIITGKGALAQLNIETGIPVNEIEPLVLFNVIFFFIAALNPGTGKFVTDEDEDE

SEQ ID NO: 20 - protein, 272 aa, Manihot esculenta
MAQTTMFMSGVSTKHVVNLKRDPLLQFQVERLRPKPLSPIFYNPLPSCSASSSKTFTTVALFRSKTKAPVKKVAEPKPKVEDGIFGTSGGIGFTKQNELFVGRVAMLGFASLLGEAITGKGILSQLNLETGIPIYEAEPLLLFFILFTLLGAIGALGDRGKFVDDPPTGLEGAVIPPGKSFRSALGLKEGGPLFGFTKANELFVGRLAQLGIAFSLIGEIITGKGALAQLNIETGIPVSEIEPLVLFNVLFFFVAALNPGTGKFVTDEDEDE

SEQ ID NO: 21 - protein, 294 aa, synthetic construct
  REGION 1..294: note = Synthetic Construct
  VARIANT 1..74: note = Xaa = Any Amino Acid; except where there are gaps in the alignment shown in Fig.10A-10E, in which case Xaa = Any Amino Acid or none
  VARIANT 149: note = Xaa = Glutamate
  VARIANT 76: note = Xaa = Any Amino Acid; except where there are gaps in the alignment shown in Fig.10A-10E, in which case Xaa = Any Amino Acid or none
  VARIANT 78..82: note = Xaa = Any Amino Acid; except where there are gaps in the alignment shown in Fig.10A-10E, in which case Xaa = Any Amino Acid or none
  VARIANT 84..95: note = Xaa = Any Amino Acid; except where there are gaps in the alignment shown in Fig.10A-10E, in which case Xaa = Any Amino Acid or none
  VARIANT 255: note = Xaa = Glutamate
  VARIANT 97..99: note = Xaa = Any Amino Acid; except where there are gaps in the alignment shown in Fig.10A-10E, in which case Xaa = Any Amino Acid or none
  VARIANT 110: note = Xaa = Any Amino Acid; except where there are gaps in the alignment shown in Fig.10A-10E, in which case Xaa = Any Amino Acid or none
  VARIANT 115: note = Xaa = Any Amino Acid; except where there are gaps in the alignment shown in Fig.10A-10E, in which case Xaa = Any Amino Acid or none
  VARIANT 126: note = Xaa = Any Amino Acid; except where there are gaps in the alignment shown in Fig.10A-10E, in which case Xaa = Any Amino Acid or none
  VARIANT 130: note = Xaa = Any Amino Acid; except where there are gaps in the alignment shown in Fig.10A-10E, in which case Xaa = Any Amino Acid or none
  VARIANT 132: note = Xaa = Any Amino Acid; except where there are gaps in the alignment shown in Fig.10A-10E, in which case Xaa = Any Amino Acid or none
  VARIANT 136..137: note = Xaa = Any Amino Acid; except where there are gaps in the alignment shown in Fig.10A-10E, in which case Xaa = Any Amino Acid or none
  VARIANT 144: note = Xaa = Any Amino Acid; except where there are gaps in the alignment shown in Fig.10A-10E, in which case Xaa = Any Amino Acid or none
  VARIANT 154: note = Xaa = Any Amino Acid; except where there are gaps in the alignment shown in Fig.10A-10E, in which case Xaa = Any Amino Acid or none
  VARIANT 168: note = Xaa = Any Amino Acid; except where there are gaps in the alignment shown in Fig.10A-10E, in which case Xaa = Any Amino Acid or none
  VARIANT 175: note = Xaa = Any Amino Acid; except where there are gaps in the alignment shown in Fig.10A-10E, in which case Xaa = Any Amino Acid or none
  VARIANT 177: note = Xaa = Any Amino Acid; except where there are gaps in the alignment shown in Fig.10A-10E, in which case Xaa = Any Amino Acid or none
  VARIANT 181: note = Xaa = Any Amino Acid; except where there are gaps in the alignment shown in Fig.10A-10E, in which case Xaa = Any Amino Acid or none
  VARIANT 183: note = Xaa = Any Amino Acid; except where there are gaps in the alignment shown in Fig.10A-10E, in which case Xaa = Any Amino Acid or none
  VARIANT 185..195: note = Xaa = Any Amino Acid; except where there are gaps in the alignment shown in Fig.10A-10E, in which case Xaa = Any Amino Acid or none
  VARIANT 198..199: note = Xaa = Any Amino Acid; except where there are gaps in the alignment shown in Fig.10A-10E, in which case Xaa = Any Amino Acid or none
  VARIANT 202..205: note = Xaa = Any Amino Acid; except where there are gaps in the alignment shown in Fig.10A-10E, in which case Xaa = Any Amino Acid or none
  VARIANT 210..212: note = Xaa = Any Amino Acid; except where there are gaps in the alignment shown in Fig.10A-10E, in which case Xaa = Any Amino Acid or none
  VARIANT 217: note = Xaa = Any Amino Acid; except where there are gaps in the alignment shown in Fig.10A-10E, in which case Xaa = Any Amino Acid or none
  VARIANT 221: note = Xaa = Any Amino Acid; except where there are gaps in the alignment shown in Fig.10A-10E, in which case Xaa = Any Amino Acid or none
  VARIANT 224: note = Xaa = Any Amino Acid; except where there are gaps in the alignment shown in Fig.10A-10E, in which case Xaa = Any Amino Acid or none
  VARIANT 229: note = Xaa = Any Amino Acid; except where there are gaps in the alignment shown in Fig.10A-10E, in which case Xaa = Any Amino Acid or none
  VARIANT 231..232: note = Xaa = Any Amino Acid; except where there are gaps in the alignment shown in Fig.10A-10E, in which case Xaa = Any Amino Acid or none
  VARIANT 234..236: note = Xaa = Any Amino Acid; except where there are gaps in the alignment shown in Fig.10A-10E, in which case Xaa = Any Amino Acid or none
  VARIANT 238: note = Xaa = Any Amino Acid; except where there are gaps in the alignment shown in Fig.10A-10E, in which case Xaa = Any Amino Acid or none
  VARIANT 243: note = Xaa = Any Amino Acid; except where there are gaps in the alignment shown in Fig.10A-10E, in which case Xaa = Any Amino Acid or none
  VARIANT 254: note = Xaa = Any Amino Acid; except where there are gaps in the alignment shown in Fig.10A-10E, in which case Xaa = Any Amino Acid or none
  VARIANT 258: note = Xaa = Any Amino Acid; except where there are gaps in the alignment shown in Fig.10A-10E, in which case Xaa = Any Amino Acid or none
  VARIANT 260..262: note = Xaa = Any Amino Acid; except where there are gaps in the alignment shown in Fig.10A-10E, in which case Xaa = Any Amino Acid or none
  VARIANT 267..269: note = Xaa = Any Amino Acid; except where there are gaps in the alignment shown in Fig.10A-10E, in which case Xaa = Any Amino Acid or none
  VARIANT 271..272: note = Xaa = Any Amino Acid; except where there are gaps in the alignment shown in Fig.10A-10E, in which case Xaa = Any Amino Acid or none
  VARIANT 276: note = Xaa = Any Amino Acid; except where there are gaps in the alignment shown in Fig.10A-10E, in which case Xaa = Any Amino Acid or none
  VARIANT 279: note = Xaa = Any Amino Acid; except where there are gaps in the alignment shown in Fig.10A-10E, in which case Xaa = Any Amino Acid or none
  VARIANT 283: note = Xaa = Any Amino Acid; except where there are gaps in the alignment shown in Fig.10A-10E, in which case Xaa = Any Amino Acid or none
  VARIANT 285: note = Xaa = Any Amino Acid; except where there are gaps in the alignment shown in Fig.10A-10E, in which case Xaa = Any Amino Acid or none
  VARIANT 287..294: note = Xaa = Any Amino Acid; except where there are gaps in the alignment shown in Fig.10A-10E, in which case Xaa = Any Amino Acid or none
XXXXXXXXXXXXXXXXXXXXXXXXXXXXXXXXXXXXXXXXXXXXXXXXXXXXXXXXXXXXXXXXXXXXXXXXXXAXFXXXXXKXXXXXXXXXXXXKXXXEDGIFGTSGGXGFTKXNELFVGRVAMXGFAXSXLGEXXTGKGILXQLNLXTGIPXYEAEPLLLFFILFXLLGAIGXLXDRGXFXDXXXXXXXXXXXVIXXGKXXXXALGLXXXGPLFXFTKXNEXFVGRXAXXGXXXSXIGEIXTGKGALAQLNXXTGXPXXXIEPLXXXNXXFFFXAAXNPGXGXFXXXXXXXX

SEQ ID NO: 22 - protein, 267 aa, Eutrema salsugineum
MAQTMMLTSGVSANQFLRNKNPLAQPKVHHLFLSGNSPVVIPSRRQSLVPLALFKPKTKAAPKKVEKVKPKVEDGIFGTSGGIGFTKANELFVGRVAMIGFAASLLGEAVTGKGILAQLNLETGIPIYEAEPLLLFFILFTLLGAIGALGDRGKFVDDPPTPTGLEKAVIAPGKNVRSALGLKEQGPLFGFTKANELFVGRLAQLGIAFSLIGEIITGKGALAQLNIETGIPIQDIEPLVLLNVAFFFFAAINPGNGKFITDDGEDM

SEQ ID NO: 23 - protein, 265 aa, Raphanus sativus
MAQTMLLTSGISANHFLRNKNPLAQPKVHHLFLSGNSPVTLPSRRPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 24 - protein, 265 aa, Brassica rapa
MAQTMLLTSGISANHFLRNKNPLAQPKVHHLFLSGNSPVTLPSRRPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFAAAINPGNGKFITDDGEES

SEQ ID NO: 25 - protein, 265 aa, Brassica oleracea
  REGION 1..265: note = var. oleracea
MDQTMLLTSGISANHFLRNKNPLAQPKVHHLFLSGNSPVTLPSRIPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFAAAINPGNGKFITDDGEES

SEQ ID NO: 26 - protein, 265 aa, Brassica napus
MAQTMLLTSGISANHFLRNKNPLAQPKVHHLFLSGNSPVTLPSRIPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFAAAINPGNGKFITDDGEES

SEQ ID NO: 27 - protein, 265 aa, Raphanus sativus
MAQTMLLTSGVSANTFLRNKNPLAQPKVHHLFLSGNSHVILPSRRPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 28 - protein, 265 aa, Brassica oleracea
MAQTMLLTSGVSANQFLRNKNPLAQPKVHHFFLSGNSHVVLPCRRPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 29 - protein, 265 aa, Brassica rapa
MAQTMLLTSGVSANQFLRNKNPLAQPKVHHFFLSGNSHVVLPSRRPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEER

SEQ ID NO: 30 - protein, 265 aa, Brassica oleracea
  REGION 1..265: note = var. oleracea
MAQTMLLTSGVSANQFLRNKNPLAQPKVHHFFLSGNSHVVLPSRRPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 31 - protein, 265 aa, Arabidopsis thaliana
MAQTMLLTSGVSAGQFLRNKSPLAQPKVHHLFLSGNSPVVLPSRRQSFVPLALFKPKTKAAPKKVEKPKKKVEDGIFGTSGGIGFTKANELFVGRVAMIGFAASLLGEALTGKGILAQLNLETGIPIYEAEPLLLFFILFTLLGAIGALGDRGQFVDDPPTGLEKAVIPPGKNVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 32 - protein, 265 aa, Arabidopsis thaliana; identical to SEQ ID NO: 1

SEQ ID NO: 33 - protein, 265 aa, Arabidopsis thaliana
MAQTFLLTSGVTAGHFLRNKSPLAQPKVHHLFLSGNSPVALPSRRQSFVPLALFKPKTKAAPKKVEKPKSKVEDGIFGTSGGIGFTKANELFVGRVAMIGFAASLLGEALTGKGILAQLNLETGIPIYEAEPLLLFFILFTLLGAIGALGDRGKFVDDPPTGLEKAVIPPGKNVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 34 - protein, 265 aa, Capsella rubella
MAQTMLLTSGVSAGQFLRNKNPLAQPKVHQLFLSGTSPVALPSRRQSFVPLALFKPKTKAAPKKVEKVKPKVEDGIFGTSGGIGFTKANELFVGRVAMIGFAASLLGEGITGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 35 - protein, 265 aa, Camelina sativa
MAQTMLLTSGVSAGQFLRNKNPLAQPKVHHLFLSGTSPVALPSRRQLFVPLALFKPKTKAAPKKVVKPKEKVEDGIFGTSGGIGFTKANELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 36 - protein, 265 aa, Camelina sativa
MAQTMLLTSGVSAGQFLRNKNPLAQPKVHHLFLSGTSPVALPSRRQSFVPLALFKPKTKAAPKKVVKPKDKVEDGIFGTSGGIGFTKANELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 37 - protein, 265 aa, Camelina sativa
MAQTMLLTSGVSAGQFLRNKNPLAQPKVHHLFLSGTSPVALPSRRQSFVPLALFKPKTKAAPKKVVKPKEKVEDGIFGTSGGIGFTKANELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 38 - protein, 270 aa, Cucurbita pepo
  REGION 1..270: note = subsp. Pepo
MAAQTMLLTSGACAGHKKEFASIFTRTLRPNTQFTHLLFNPSPSHSVSLPARGFTTLAVFKSRTKVPPKKVAKPKVEDGIFGTSGGIGFTKQNELFVGRVAMIGFAASLLGEGITGKGILAQLNLETGVPIYEAEPLLLFFILFTLLGAIGALGDRGKFIDDPETAGLERAVIPPGKGFRSALGLKEGGPLFGFTKANELFVGRLAQLGFAFSLIGEIITGKGALAQLNIETGVPINEIEPLVLLNVVFFFIAALNPGTGKFVTDEEDDD

SEQ ID NO: 39 - protein, 270 aa, Cucurbita moschata
MAAQTMLLTSGACAGHKIDFASIFTPTLRPNAQVTRLLFNPSPPHSVSLPARGFTTLAAFKSRTKVPPKKVSKPKVEDGIFGTSGGIGFTKQNELFVGRVAMIGFAASLLGEGITGKGILAQLNLETGVPIYEAEPLLLFFILFTLLGAIGALGDRGKFIDDPETAGLERAVIPPGKSFRSALGLKEGGPLFGFTKANELFVGRLAQLGFAFSLIGEIITGKGALAQLNIETGVPINEIEPLVLLNVVFFFIAALNPGTGKFVTDEEDDD

SEQ ID NO: 40 - protein, 270 aa, Cucurbita maxima
MAAQTMLLTSGACAGHKREFASIFTQTLRPNAQVTRLLFNPSPSHSVTLPARGFTTLAAFKSRTKVPPKKVSKPKVEDGIFGTSGGIGFTKQNELFVGRVAMIGFAASLLGEGITGKGILAQLNLETGVPIYEAEPLLLFFILFTLLGAIGALGDRGKFIDDPETAGLERAVIPPGKSFRSALGLKEGGPLFGFTKANELFVGRLAQLGFAFSLIGEIITGKGALAQLNIETGVPINEIEPLVLLNVVFFFIAALNPGTGKFVTDEEDDD

SEQ ID NO: 41 - protein, 273 aa, Momordica charantia
MAAQTMLLTSGVCASHGVGAKREAAALAQRLRPNAPLARLFFDPSPLPCSPARGLTTLAVFKSRTKATPKKVVKPKQKVEDGIFGTSGGIGFTKQNELFVGRVAMIGFAASLLGEGITGKGILAQLNLETGVPIYEAEPLLLFFILFTLLGAIGALGDRGKFIDDPETAGLERAVIPPGKGFRSALGLKEGGPLFGFTKANELFVGRLAQLGFAFSLIGEIITGKGALAQLNIETGVPINEIEPLVLLNVVFFFIAALNPGTGKFVTDEEEDE

SEQ ID NO: 42 - protein, 272 aa, Cucumis sativus
MAAQTMLLTSGVCVGHGVCLKRELSLRPNYTQFTRLFFNPLPSHSVSLPPRGFTTLAVFKSRTKAPPKKVEKPKQKVEDGIFGTSGGIGFTKQNELFVGRVAMIGFAASILGEAITGKGILAQLNLETGIPIYEAEPLLLFFILFTLLGAIGALGDRGKFIDDPEPATGLERAVIPPGKSFRSALGLKEGGPLFGFTKANELFVGRLAQLGFAFSLIGEIITGKGALAQLNIETGVPINEIEPLVLLNVVFFFIAAVNPGTGKFVTDEEDDE

SEQ ID NO: 43 - protein, 272 aa, Cucumis melo
MAAQTMLLTSGVCVGHGVCLKRELSLRPNNNQFTRLFFNPLPNHSVSLPARGFTPLAVFKSRTKAPPKKVEKPKQKVEDGIFGTSGGIGFTKQNELFVGRVAMIGFAASLLGEAITGKGILAQLNLETGIPIYEAEPLLLFFILFTLLGAIGALGDRGKFIDDPEPATGLERAVIPPGKSFRSALGLKEGGPLFGFTKANELFVGRLAQLGFAFSLIGEIITGKGALAQLNIETGVPINEIEPLVLLNVVFFFIAAVNPGTGKFVTDEEDDE

SEQ ID NO: 44 - protein, 275 aa, Cucurbita moschata
MAAQTMVLSSGVCASHGVALKREIASLTQRLRPNAQFSRLFFNPLPSSSVSLPARGFRTLAIFKSKAKVPAKKVVKPKEKVEDGIFGTSGGIGFTKQNELFVGRVAMIGFAASLLGEGITGKGILAQLNLETGVPIYEAEPLLLFFILFTLLGAIGALGDRGKFIDDPEPATGLERAVIPPGKGLRSALGLKEGGPLFGFTKANELFVGRLAQLGFAFSLIGEIITGKGALAQLNIETGVPINEIEPLVLLNVLFFFIAAVNPGTGKFVTDEEED

SEQ ID NO: 45 - protein, 275 aa, Cucurbita maxima
MAAQTMVLSSGVCASHGVALKREIASLTRRLRPNAQSSRLFFNPLPSSSVSLPARGFRTLAIFKSKAKVPAKKVVKPKEKVEDGIFGTSGGIGFTKQNELFVGRVAMIGFAASLLGEGITGKGILAQLNLETGVPIYEAEPLLLFFILFTLLGAIGALGDRGKFIDDPEPATGLERAVIPPGKGLRSALGLKEGGPLFGFTKANELFVGRLAQLGFAFSLIGEIITGKGALAQLNIETGVPINEIEPLVLLNVLFFFIAAVNPGTGKFVTDEEED

SEQ ID NO: 46 - protein, 277 aa, Abrus precatorius
MAQTMVLMSSVPASNSMDLKGNSLLQLRSQRLRPKFSRHFFNPLPSTFSFPSSSSTFTTIAIYKTKTKASPTSKVVKSKPKSKVEDGIFGTSGGFGFTKQNELFVGRVAMLGFAASLLGEAITGKGILAQLNLETGIPIYEAEPLLLFFIIFTLLGAIGALGDRGQFVDDPPTPLDKPFIPPGKGFRAALGLREGGPLFGFTKANELFVGRLAQMGFAFSLIGEIITGKGALAQLNIETGVPINEIEPLVLFSIVFFFFAALNPGTGHFVTDEGEEV

SEQ ID NO: 47 - protein, 269 aa, Phaseolus vulgaris
MAQTMFLMSSVSSSHSLDLKKDLFLQLRPKFSQLSFSPLPSSASFSSSPRTFTTLALFKSKTKAPPAKTKVTKSKPKVEDGVFGTSGGFGFTKQNELFVGRVAMLGFAASLLGEAVTGKGILSQLNLETGIPIYEAEPLLLFFILFTLLGAIGGLGDRGKFVDDDEPNTAGVIPSGKGFREAIGLGSGPLFGFTKANELFVGRLAQLGFVFSLIGEIITGKGALAQLNIETGVPINEIEPLVLFNVLFFFVAALNPGTGKFVTDEGDDE

SEQ ID NO: 48 - protein, 265 aa, Vigna radiata
  REGION 1..265: note = var. radiata
MAQTMLLMSSVSSSHSLGLKKDLFQQLRPKFSQLSFNPLPSSSSFSSSPRTFTTLALFKSKTKAPPAKVVKPKQKVEDGIFGTSGGFGFTKQNELFVGRVAMLGFAASLLGEAVTGKGILSQLNLETGIPIYEAEPLLLFFILFTLLGAIGGLGDRGKFVDDEPNTGGVIPSGKGFREALGLGSGRLFGFTKANELFVGRLAQLGFVFSLIGEIITGKGALAQLNIETGVPINEIEPLVLFNVLFFFVAALNPGTGKFVTDEEDD

SEQ ID NO: 49 - protein, 265 aa, Vigna angularis
MAQTMLLMSSVSSSHSLGLKKELFQQLRPKFSQLSFNPLPSSSSFSSSPRTFTTLALFKSKTKAPPAKTKVTKPKVEDGVFGTSGGFGFTKQNELFVGRVAMLGFAASLLGEAVTGKGILSQLNLETGIPIYEAEPLLLFFILFTLLGAIGGLGDRGKFVDDEPNTGGVIPAGKGFREALGLGSGPLFGFTKANELFVGRLAQLGFVFSLIGEIITGKGALAQLNIETGVPINEIEPLVLFNVLFFFIAALNPGTGKFVTDEEDD

SEQ ID NO: 50 - protein, 270 aa, Cajanus cajan
MAQTMLLMSSVSSSYSVALNKDVFLQLQAQRLRPKFSDLSFNPLPSNSLFSSRPTFTTLALFKSKTKAPPAKTKVTKPKVEDGIFGTSGGFGFTKQNELFVGRVAMLGFAASLLGEAVTGKGILAQLNLETGIPIYEAEPLLLFFILFTLLGAIGGLGDRGRFVDDEPTTAGVIPPGKGFREALGLGQGPLFGFTKSNELFVGRLAQLGFVFSLIGEIITGKGALAQLNIETGVPINEIEPLVLFNVLFFFIAALNPGTGKFVTDEEADD

SEQ ID NO: 51 - protein, 273 aa, Abrus precatorius
MAQTMVLMSSVSSSYSVDLKKYPLLQLQSQRLRPKFSHVSFNPLPSNSLPFSSRTFTTLALFKSKTKSAPPKTKVTKPKPKVEDGVFGTSGGFGFTKQNELFVGRVAMLGFAASLLGEGLTGKGILAQLNLETGIPIYEAEPLLLFFILFTLLGAIGALGDRGKFVDDDPPAGAVIPPGKGVRGALGLSEGGPLFGFTKANELFVGRLAQLGFAFSLIGEIITGKGALAQLNIETGVPINEIEPLVLFNVLFFFIAALNPGTGKFLTDEEEDD

SEQ ID NO: 52 - protein, 271 aa, Mucuna pruriens
MAQTMLLMSSVSSSYSVDLKKDLFLQLQSQRLRPKFSHISFNPLPSNSFFSSPRTFTTQALFKSKTKSPPPKTKVTKPKPKVEDGVFGTSGGFGFTKQNELFVGRVAMLGFAASLLGEGITGKGILAQLNLETGIPIYEAEPLLLFFILFTLLGAIGALGDRGQFVDDEPTTGGVIPPGKGLREALGLGQGPLFGFTKANELFVGRLAQLGFAFSLIGEIITGKGALAQLNIETGVPINEIEPLVLFNVLFFFVAALNPGTGKFVTDEEED

SEQ ID NO: 53 - protein, 273 aa, Glycine soja; identical to SEQ ID NO: 8

SEQ ID NO: 54 - protein, 271 aa, Arachis ipaensis
MMAQTMLLMSSVSTTYSMDLKKDTLLQLQNQRLRPKFSHLPFNPIPSNSLSFSPPTFTTLALFKSKTKAPPKVVKPKPKVEDGIFGTSGGFGFTKQNELFVGRVAMIGFAASILGEAITGKGILAQLNLETGVPIYEAEPLLLFFILFTLLGAIGALGDRGKFVDDDEPNTGGVIPPGKGFRSALGLKEGGPLFGFTKANELFVGRLAQLGFAFSLIGEIITGKGALAQLNIETGVPINEIEPLVLFNVAFFFIAALNPGTGKFVTDEEED

SEQ ID NO: 55 - protein, 271 aa, Arachis hypogaea
MMAQTMLLMSSVSTTYSMDLKKDTLLQLQNQRLRPKFSHLPFNPIPSNSLSFSPPTFTTLALFKSKTKAPPKVVKPKPKVEDGIFGTSGGFGFTKQNELFVGRVAMLGFAASILGEAITGKGILAQLNLETGIPIYEAEPLLLFFILFTLLGAIGALGDRGRFVDDDEPNTGGVIPPGKGFRSALGLKEGGPLFGFTKANELFVGRLAQLGFAFSLIGEIITGKGALAQLNIETGVPINEIEPLVLFNVAFFFIAALNPGTGKFVTDEEED

SEQ ID NO: 56 - protein, 270 aa, Pisum sativum
MAQTMLLMSSVSSTYSVPLNKDPLLQLQCQRLKPRFSDISFSPLSSNSKSFSSRTFKTLALFKSKTKAPAKVVPKQKPKVEDGVFGTSGGFGFTKQNELFVGRVAMIGFAASILGEGITGKGILSQLNLETGIPIYEAEPLLLFFIIFTLLGAIGALGDRGKFVDDEPTTGGVIPPGKGFRQALGLSEGGPLFGFTKSNELFVGRLAQLGFAFSLIGEIITGKGALAQLNIETGVPINEIEPLVLFNVAFFFIAALNPGTGKFVTDEEED

SEQ ID NO: 57 - protein, 269 aa, Cicer arietinum
MAQTMLLMSSVSSTYSVDLKKDPLLQLQCQRLRPKFSDISFNPLNSNSKSFSSRTFTTLALFKSKAKAPPKVVKPKPKVEDGVFGTSGGFGFTKQNELFVGRVAMIGFAASILGEAVTGKGILAQLNLETGIPIYEAEPLLLFFIIFTLLGAIGALGDRGKFVDDEPNTGGVIPPGKGFRSALGLSEGGPLFGFTKSNELFVGRLAQLGFAFSLIGEIITGKGALAQLNIETGVPINEIEPLVLFNVAFFFVAALNPGTGQFVKDEDDD

SEQ ID NO: 58 - protein, 270 aa, Medicago truncatula
MAQTMLLMSSVSSTYSVDLKKDPLLQLQCQRLRPRFSDVSFNPLPSNSKCFSSRTFTTLALFKSKTKAPAKVVKKPKPKVEDGIFGTSGGFGFTKQNELFVGRVAMIGFAASILGEALTGKGILAQLNLETGIPIYEAEPLLLFFIIFTLLGAIGALGDRGKFVDDEPNTGGVIPPGKGFRSALGLSEGGPLFGFTKSNELFVGRLAQLGFVFSLIGEIITGKGALAQLNIETGVPITEIEPLVLFNVIFFFIAALNPGTGTFVTDDEED

SEQ ID NO: 59 - protein, 269 aa, Trifolium pratense
MAQTMLLMSSVSSTYSVELKKDPLLQLQCQRLRPRISDVSFNPLSSNSKRLSSRTFTTLALFKSKTKAPAKVVKPKPKVEDGIFGTSGGFGFTKQNELFVGRVAMIGFAASILGEALTGKGILAQLNLETGIPIYEAEPLLLFFIIFTLLGAIGALGDRGKFVDDEPTTGGVIPPGKGFRAALGLREGGPLFGFTKSNELFVGRLAQLGFVFSLIGEIITGKGALAQLNIETGVPITEIEPLVLFNVIFFFVAALNPGTGTFVKDEDED

SEQ ID NO: 60 - protein, 269 aa, Trifolium subterraneum
MAQTMLLMSSVSSTYSVELKKDPLLQLQCQRLRSRISDVSFNPLSSNSKSLSSRTFTTLALFKSKTKAPAKVVKPKPKVEDGIFGTSGGFGFTKQNELFVGRVAMIGFAASILGEALTGKGILAQLNLETGIPIYEAEPLLLFFIIFTLLGAIGALGDRGKFVDDEPTTGGVIPPGKGFRAALGLREGGPLFGFTKSNELFVGRLAQLGFVFSLIGEIITGKGALAQLNVETGVPITEIEPLVLFNVIFFFIAALNPGTGTFVTDEEED

SEQ ID NO: 61 - protein, 273 aa, Lupinus angustifolius
MAQSIMLMSSVSSSYSVSLKKDPLLQLQSQKLRPKFSHLSFNPLPNNSLHFSSHTFTTLALFKSKTKAPPKKVEKPKQKVEDGIFGTSGGIGFTKQNELFVGRVAMIGFAASLLGEGLTGKGILSQLNLETGVPIYEAEPLLLFFILFTLLGAIGALGDRGKFVDDEPATGLDKAIIPPGKGLRGVLGLQEGGPLFGFTKSNELFVGRLAQLGFAFSLIGEIITGKGALAQLNIETGVPVNEIEPLVLFNVLFFFIAALNPGTGKFVTDEEED

SEQ ID NO: 62 - protein, 273 aa, Lotus japonicus
MAQTMLLMSSVSSSYSVDLKKDPLLHLQSQRLRPKFSQLSFNPLPSNSSLFSSRTFTTLALFKSKAKAPPPKVVKQKPKVEDGVFGTSGGIGFTKQNELFVGRVAMIGFAASLLGEALTGKGILAQLNLETGIPIYEAEPLLLFFILFTLLGAIGALGDRGKFVDDESPTGLDKAVIPPGKGLRGALGLSEGGPLFGFTKSNELFVGRLAQLGFAFSLIGEIITGKGALAQLNIETGIPINEIEPLVLFNVAFFFIAALNPGTGKFVSDDGED

SEQ ID NO: 63 - protein, 274 aa, Spinacia oleracea
MAQAMLLMMPGVSTTNTIDLKRNALLKLQIQKIKPKSSTSNLFFSPLPSSSSSSSTVFKTLALFKSKAKAPKKVEKPKLKVEDGLFGTSGGIGFTKENELFVGRVAMIGFAASLLGEGITGKGILSQLNLETGIPIYEAEPLLLFFILFTLLGAIGALGDRGRFVDEPTTGLEKAVIPPGKDVRSALGLKTKGPLFGFTKSNELFVGRLAQLGFAFSLIGEIITGKGALAQLNIETGVPINEIEPLVLLNVVFFFIAAINPGTGKFITDDEEED

SEQ ID NO: 64 - protein, 271 aa, Beta vulgaris
  REGION 1..271: note = subsp. Vulgaris
MAQAMLLMPSVSTTNTIDLKRNALLKIQIQKIKPKSSSSHLFFSPLSSSSSSSSTFKTLALFKPKTKAPVKKVEKPKLKVEDGLFGTSGGIGFTKENELFVGRVAMIGFAASLLGEGLTGKGILAQLNLETGIPIYEAEPLLLFFILFTLLGAIGALGDRGRFVDDPPTGLEKAVIPPGKNVRSALGLREGGPLFGFTKSNELFVGRLAQLGFAFSLIGEIITGKGALAQLNIETGVPVNEIEPLVLLNVLFFFIAAINPGTGKFITDEEE

SEQ ID NO: 65 - protein, 273 aa, Chenopodium quinoa
MAQAMLLMPNVSTNTVDLKRNSLLKLQIEKIKPKSSSSHLFFSPLPSLSSSSSSTVLKTFALFKSKAKAPVKKVEKPKLKVEDGLFGTSGGIGFTKENELFVGRVAMIGFAASLLGEAVTGKGILAQLNLETGIPIYEAEPLLLFFILFTLLGAIGALGDRGRFVDDPPTGLEKAVIPPGKGVRSALGLKEGGPLFGFTKSNELFVGRLAQLGFAFSLIGEIITGKGALSQLNIETGVPINEIEPLVLLNVVFFFIAAINPGTGKFITDEEED

SEQ ID NO: 66 - protein, 273 aa, Chenopodium quinoa
MAQAMLLMPNVSTNTVDLKRNSLLKLQIEKIKPKSSSSHLFFSPLPSLSSSSSSNVLKTFALFKSKAKAPAKKVEKPKLKVEDGLFGTSGGIGFTKENELFVGRVAMIGFAASLLGEGITGKGILAQLNLETGIPIYEAEPLLLFFILFTLLGAIGALGDRGRFVDDPPTGLEKAVIPPGKNVRSALGLKEGGPLFGFTKSNELFVGRLAQLGFAFSLIGEIITGKGALAQLNIETGVPINEIEPLVLLNVVFFFIAAINPGTGKFITDEEED

SEQ ID NO: 67 - protein, 266 aa, Eucalyptus grandis
MAQTMMLMPSVSGHSVDLRTPRLRPTSSSFSRSLLYNPLPSPAASSHRSSPVFALFKSKTKAPPKKVVKEKPKVEDGVFGTSGGIGFTKENELFVGRVAMIGFAASLLGEAITGKGILAQLNLETGVPIYEAEPLLLFFILFTLLGAIGALGDRGRFVDDSAGIEGAVIPPGKGFLSALGLKEGGPLFGFTKANELFVGRLAQLGFAFSLIGEIITGKGALAQLNIETGVPISEIEPLVLFNVLFFFIAALNPGTGKFLTDEDLED

SEQ ID NO: 68 - protein, 276 aa, Rosa chinensis
MAQTMLLMSSSSVAKTSVVDLKSSDPLLQLQVQRLRPKSFSQLVFRPLPSSTTSLRSSGVVALFKSKTKAPASKKAPSPKPKVESGIFGTSGGIGFTKENELFVGRVAMIGFAASLLGEAITGKGILSQLNLETGVPIYEAEPLLLFFILFTLLGAIGALGDRGSFVDDPPTGIEGAVIPPGKGIRGALGLKEGGPLFGFTKANELFVGRLAQLGFAFSLIGEIITGKGALAQLNIETGVPINEIEPLVLLNVVFFFLAAVNPGTGKFITDEGEEE

SEQ ID NO: 69 - protein, 273 aa, Fragaria vesca
  REGION 1..273: note = subsp. Vesca
MAQTMLLMSSSSVAKTNVVDLKSSDPVLQLQVQRLRPKSFSQLVFRPLPSSTTSLRSSGVVALFKSKTKAPVKKASPPKPKVESGIFGTSGGIGFTKENELFVGRVAMIGFAASLLGEAITGKGILQQLNLETGIPIYEAEPLLLFFILFTLLGAIGALGDRGQFVDDTPGIEGAVIPPGKGLKSALGLKEGGPLFGFTKANELFVGRLAQLGFAFSLIGEIITGKGALAQLNIETGVPINEIEPLVLLNVVFFFLAAVNPGTGKFITDEDEE

SEQ ID NO: 70 - protein, 279 aa, Prunus persica
MAQTMLLMSSSVSSSHVVDLKSSDPLLQLQVQRLRPKSFSQLVFRPLPSSSSFASSSSPAIPTIVALFKSKTKAPVKKAPAPKPKVEDGIFGTSGGIGFTKQNELFVGRVAMIGFAASLLGEAITGKGILAQLNLETGVPIYEAEPLLLFFILFTLLGAIGALGDRGSFVDDPPAGIERAVIPPGKGFRSALGLKEGGPLFGFTKANELFVGRLAQLGFAFSLIGEIITGKGALAQLNIETGVPINEIEPLVLLNVVFFFIAALNPGTGKFVTDEGEEE

SEQ ID NO: 71 - protein, 279 aa, Prunus mume
MAQTMLLMSSSVSSRHAVDLKSSDPLLQLQVQRLRPKSFSQLVFRPLPSSSSFASSSSPAIPTVVALFKSKTKAPVKKASPPKPKVEDGIFGTSGGIGFTKQNELFVGRVAMIGFAASLLGEAVTGKGILAQLNLETGVPIYEAEPLLLFFILFTLLGAIGALGDRGSFVDDPPTGIEGAVIRPGKGFRSALGLKEGGPLFGFTKANELFVGRLAQLGFAFSLIGEIITGKGALAQLNIETGVPINEIEPLVLLNVVFFFIAALNPGTGKFVTDEGEEE

SEQ ID NO: 72 - protein, 279 aa, Prunus avium
MAQTMLLMSSSVSSSHVVDLKSSDPLLQLRVQRLRPKSFSQLVFRPLPSSSSFASSSSPAISTVVALFKSKTKAPVKKATPAKPKVEDGIFGTSGGIGFTKQNELFVGRVAMIGFAASLLGEAVTGKGILAQLNLETGVPIYEAEPLLLFFILFTLLGAIGALGDRGSFVDDPPTGIEGAVIRPGKGFRSALGLKEGGPLFGFTKANELFVGRLAQLGFAFSLIGEIITGKGALAQLNIETGVPINEIEPLVLLNVVFFFIAALNPGTGKFVTDEGEEE

SEQ ID NO: 73 - protein, 279 aa, Prunus yedoensis
  REGION 1..279: note = var. nudiflora
MAQTMLLMSSSVSSSHVVDLKSSDPLLQLRVQRLRPKSFSQLVFGPLPSSSSFASSSSPAISTVVALFKSKTKAPVKKASPAKPKVEDGIFGTSGGIGFTKQNELFVGRVAMIGFAASLLGEAVTGKGILAQLNLETGVPIYEAEPLLLFFILFTLLGAIGALGDRGSFVDDPPTGIEGAVIRPGKGFRSVLGLKEGGPLFGFTKANELFVGRLAQLGFAFSLIGEIITGKGALAQLNIETGVPINEIEPLVLLNVVFFFIAALNPGTGKFITDEGEEE

SEQ ID NO: 74 - protein, 279 aa, Pyrus x bretschneideri
MAQTMLLMSSSVSSGHVVDLKSSDPLLQVQAQRLRPKSFSQLVCRPLPSSSFAASSSSTRILTIVALFKSKTKAPAKKAPPPKPKVEDGIFGTSGGIGFTKQNELFVGRVAMIGFAASLLGEAITGKGILSQLNLETGIPIYEAEPLLLFFILFTLLGAIGALGDRGRFVDDPPAGIEGAVIPPGKGLKSALGLKEGGPLFGFTKANELFVGRLAQLGFAFSLIGEIITGKGALAQLNIETGVPISEIEPLVLLNVVFFFIAAVNPGTGKFITDVDEED

SEQ ID NO: 75 - protein, 278 aa, Malus domestica
MAQTMLLMSSSVSSGHVVDLKSSDPLLQVQAQRLRPKSFSQLVFRPLPSSSSASSSSTRISTVVALFKSKTKAPAKKAPPPKPKVEDGIFGTSGGIGFTKQNELFVGRVAMIGFAASLLGEAITGKGILSQLNLETGIPIYEAEPLLLFFILFTLLGAIGALGDRGRFVDDPPAGIEGAVIPPGKGLKSALGLKEGGPLFGFTKANELFVGRLAQLGFAFSLIGEIITGKGALAQLNIETGVPISEIEPLVLLNVVFFFIAAVNPGTGKFITDVDEED

SEQ ID NO: 76 - protein, 278 aa, Malus domestica
MAQTMLLMSSSVSSGHVVDLKSSDPLLQVQAQRLRPKSFSQLVFRALPSSSSASSSSTRISTVVALFKSKTKAPAKKAPPPKPKVEDGIFGTSGGIGFTKQNELFVGRVAMIGFAASLLGEAITGKGILSQLNLETGIPIYEAEPLLLFFILFTLLGAIGALGDRGRFVDDPPAGIEGAVIPPGKGLKSALGLKEGGPLFGFTKANELFVGRLAQLGFAFSLIGEIITGKGALAQLNIETGVPISEIEPLVLLNVVFFFIAAVNPGTGKFITDVDEED

SEQ ID NO: 77 - protein, 278 aa, Malus domestica
  VARIANT 261: note = Xaa = Any Amino Acid
  VARIANT 266: note = Xaa = Any Amino Acid
MLLMSSSVSSGHVVDLKSSDRLLQVQVQRLRPKSFSQLVFRPLPSSSFASSSSTVVALFKSKTKAPAKKAPSPKPKVEDGIFGTSGGIGFTKQNELFVGRVAMIGFAASLLGEAITGKGILSQLNLETGVPIYEAEPLLLFFILFTLLGAIGALGDRGKFVDDPPTGIEGAVIPPGKGFKSALGLNEGGPLFGFTKANELFVGRLAQLGFAFSLIGEIITGKGALAQLNIETGVPINEIEPLVLLNVVFFFIAAVNPGTGXFITDXMKKTRERETIHH

SEQ ID NO: 78 - protein, 274 aa, Pyrus x bretschneideri
MAQTMLLMSSSVSSGHVVDLKSSDPLLKVQVQRLRPKSFSQLVFRPLPSSSFASSSSTVVALFKSKTKAPAKKAPSPKPKVEDGIFGTSGGIGFTKQNELFVGRVAMIGFAASLLGEAITGKGILSQLNLETGVPIYEAEPLLLFFILFTLLGAIGALGDRGRFVDDPPTGIEGAVIRPGKGLKSALGLNEGGPLFGFTKANELFVGRLAQLGFAFSLIGEIITGKGALAQLNIETGVPINEIEPLVLLNVVFFFIAAVNPGTGKFITDVDEED

SEQ ID NO: 79 - protein, 274 aa, Malus domestica
MAQTMLLMSSSVSSGHVVDLKSSDRLLQVQVQRLRPKSFSQLVFRPLPSSSFASSSSTVVALFKSKTKAPAKKAPSPKPKVEDGIFGTSGGIGFTKQNELFVGRVAMIGFAASLLGEAITGKGILSQLNLETGVPIYEAEPLLLFFILFTLLGAIGALGDRGKFVDDPPTGIEGAVIPPGKGFKSALGLNEGGPLFGFTKANELFVGRLAQLGFAFSLIGEIITGKGALAQLNIETGVPINEIEPLVLLNVVFFFIAAVNPGTGKFITDVDEED

SEQ ID NO: 80 - protein, 280 aa, Morus notabilis
MAQTMLLSSSSVSTSHALDLQKHPLLHLQVQSLKPKPFSHLFFSPLSSRNPSLASSSSRGISTVFALFKSKTKAAPTKKVVKEKPKVEDGIFGTSGGIGFTKQNELFVGRVAMIGFAAALLGEAITGKGILSQLNLETGIPIYEAEPLLLFFILFTLLGAIGALGDRGRFVDDPPTGIEGAVIPPGKSLRSALGLKEGGPLFGFTKANELFVGRLAQLGFAFALIGEIITGKGALAQLNVETGVPINEIEPLVLLNVLFFFIAAVNPGTGKFITDENEED

SEQ ID NO: 81 - protein, 275 aa, Trema orientale
MAQTMLLSSSSVSNCHVVDLKRDPLLQIRVQRLKTTKPFSQSLTFNPLTSSSKSPAISTVFALFKSKTKAAPKKVVKSKPKVEDGVFGTSGGIGFTKQNELFVGRVAMIGFAASILGEAITGKGILAQLNLETGIPIYEAEPLLLFFILFTLLGAIGALGDRGQFVDDPPTGIEGAVIPPGKGFRSALGLKEGGPLFGFTKANELFVGRLAQLGFAFSLIGEIITGKGALAQLNIETGVPINEIEPLVLFNVLFFFIAAVNPGTGKFVTDVDEED

SEQ ID NO: 82 - protein, 275 aa, Parasponia andersonii
MAQTMLLSSSSVSNCHVVDLKRDPLLQIRVQRLKTTKPFSQSFTFNPLTSSSKSPAMSTVFALFKSKTKAAPKKVVKSKPKVEDGVFGTSGGIGFTKQNELFVGRVAMVGFAASILGEAITGKGILAQLNLETGIPIYEAEPLLLFFILFTLLGAIGALGDRGQFVDDPPTGIEGAVIPPGKGFRSALGLKEGGPLFGFTKANELFVGRLAQLGFAFSLIGEIVTGKGALAQLNIETGVPINEIEPLVLFNVLFFFIAAVNPGTGKFVTDVDEED

SEQ ID NO: 83 - protein, 277 aa, Ziziphus jujuba
MAQTMLLMSSSSVSSSVDLKKRSDPLLQFQIQRLRPTKPLSHLFFKSLPSSSSSSPPVSTVVALFKSKTKAPPKKAVKAPKPKVEDGVFGTSGGIGFTKQNELFVGRVAMLGFAASILGEAITGKGILAQLNLETGIPIYEAEPLLLFFILFTLLGAIGALGDRGQFVDDPPTGIEGAVIPPGKSFRSALGLKEGGPLFGFTKANELFVGRLAQLGFAFSLIGEIITGKGALAQLNIETGVPINEIEPLVLFNVIFFFFAALNPGTGKFVTDVDEED

SEQ ID NO: 84 - protein, 271 aa, Aquilegia coerulea
MAQTMMLMSSVAGHGLDLKRVPSLQTLKPKPFAHFLLPPSSQRYQLAKYSSSTTTIALFKSRTKAPAKTKQAPKPKPKVEDGIFGTSGGIGFTKQNELFVGRVAMLGFAASLLGEGITGKGILAQLNIETGIPIYEAEPLLLFFILFTLLGAIGALGDRGRFVDDPPTGLEKAVIPPGKGIKSALGLKEGGPLFGFTKANELFVGRLAQLGFVFSLIGEIITGKGALAQLNIETGVPLNELEPLVLFNVIFFFFAALNPGNGKFVTDEDEE

SEQ ID NO: 85 - protein, 273 aa, Macleaya cordata
MAQTMMLMSNVGTAGGHGLNLKRESLLQRLKPKPFSHLLLPPLSTHQLSSTSSSTSTTPFALFKSKTKAPVKKAPAPKPKVEDGIFGTSGGIGFTKQNELFVGRVAMLGFAASLLGEAITGKGILSQLNLETGIPIYEAEPLLLFFILFTLLGAIGALGDRGKFVDDPPTGLERAVIPPGKSFRSALGLKEGGPIFGFTKSNELFVGRLAQLGIAFSIIGEIITGKGALAQLNIETGVPINEIEPLVLFNVVFFFIAALNPGNGKFVTDDEEE

SEQ ID NO: 86 - protein, 272 aa, Papaver somniferum
MAQSMMLMSNVAGQGLNFKREALVDRLKPKPFSHFLLPSSLSSPSTSSSSKAFTIFALFKSKTKAPAKKAAPAPKPKVEDGIFGTSGGIGFTKQNELFVGRVAMLGFAASLLGEAITGKGILSQLNLETGIPIYEAEPLLLFFILFTLLGAIGALGDRGKFVDDAPATGIDRAVIPPGKGFRAALGLKEGGPVFGFTKSNELFVGRLAQLGVAFSIIGEIITGKGALAQLNIETGVPIGEIEPLVLFNVVFFFIAALNPGSGKFVTDDGEEE

SEQ ID NO: 87 - protein, 272 aa, Papaver somniferum
MAQSMMLMSNVAGQGLNFKREALVDRLKPKPFSHFLLPSSLSSPSTSSSSKTFTTFALFKSKTKAPAKKAAPAPKPKVEDGIFGTSGGIGFTKQNELFVGRVAMLGFAASLLGEAITGKGILSQLNLETGIPIYEAEPLLLFFILFTLLGAIGALGDRGKFVDDAPATGIDRAVIPPGKGFRAALGLKEGGPVFGFTKSNELFVGRLAQLGVAFSIIGEIITGKGALAQLNIETGVPIGEIEPLVLFNVVFFFIAALNPGSGKFVTDDGEEE

SEQ ID NO: 88 - protein, 272 aa, Daucus carota
  REGION 1..272: note = subsp. Sativus
MAQTMLLMSSSTCVSGHAADNLKSPALPLLQRLKPKPFSHSLLPHLANISKSSSTFTTYALFKSKAKAPAKKVVAPKPKVEDGIFGTSGGIGFTKQNELFVGRVAMIGFAASLLGEAVTGKGILAQLNLETGIPIYEAEPLLLFFILFTLLGAIGALGDRGRFVDDPPTGLEKAVIKPGKGFRGALGLSEGGPLFGFTKSNELFVGRLAQLGIAFSLIGEIITGKGALAQLNIETGIPISDIEPLVLLNVVFFFVAALNPGTGKFITDEEED

SEQ ID NO: 89 - protein, 259 aa, Actinidia chinensis
  REGION 1..259: note = var. chinensis
MAQTMFLTSSVCGHALVQRLRPKSFSHISQSPIPTSSSIKPNTIVALFKSKTKAPLPAKKAPKTASKVEDGIFGTSGGIGFTKQNELFVGRVAMIGFAASLLGEAITGKGILAQLNLETGIPIYEAEPLLLFFILFTLLGAIGALGDRGRFVDDPPTGLEKAVIPPGKGVRSALGLKEGGPLFGFTKANELFVGRLAQLGIAFSLIGEIITGKGALAQLNIETGVPISEIEPLLLFNVAFFFFAALNPGTGKFVTDEEE

SEQ ID NO: 90 - protein, 260 aa, Actinidia chinensis
  REGION 1..260: note = var. chinensis
MAQTMFLTSSVCGHALVQRLRPKSFSHISQSPIPTSSSSIKTNTIVALFKSKTKAPPPAKKAPKVASKVEDGIFGTSGGIGFTKQNELFVGRVAMIGFAASLLGEAITGKGILAQLNLETGIPIYEAEPLLLFFILFTLLGAIGALGDRGRFVDDPPTGLEKAVIPPGKGIRSALGLKEGGPLFGFTKANELFVGRLAQLGIAFSLIGEIITGKGALAQLNIETGVPISEIEPLLLFNVAFFFFAALNPGTGKFLTDEEE

SEQ ID NO: 91 - protein, 262 aa, Lactuca sativa
MAQAMLLTSNTILSGQPLLQSLKPKPFSYHLLPRNLPNLSPATKFTSPVALFKSKAKAPVKKAVVSKPKVEDGIFGTSGGIGFTKQNELFVGRVAMIGFAASLLGEGITGKGILQQLNLETGIPIYEAEPLLLFFILFTLLGAIGALGDRGRFVDEPTTGLDKAVIPPGKGVRGALGLKEGGPLFGFTKANELFVGRLAQLGIAFSLIGEIITGKGALAQLNIETGIPISDIEPLVLFNVAFFFIAALNPGTGKFVTDEEED

SEQ ID NO: 92 - protein, 264 aa, Artemisia annua
MAQTMLLTSNTIISGSPLLQSLRPKPFAHLLLPPSLASSSSTRTYTSPIALFKSKAKAAPAKKAAPAPKPKVEDGIFGTSGGIGFTKQNELFVGRVAMIGFAASLLGEAITGKGILQQLNLETGIPIYEAEPLLLFFILFTLLGAIGALGDRGRFVDDAPTGLDKAVIAPGKGFRSALGLSEGGPLFGFTKSNELFVGRLAQLGIAFSLIGEIITGKGALAQLNIETGLPISEIEPLVLFNVAFFFVAALNPGTGKFVTDEDED

SEQ ID NO: 93 - protein, 264 aa, Helianthus annuus
MAQTMLLTSNTIISGQPLLRSLRPKPFSYIVLPPSLLPKSSPTSTTFSSPVALFKPKTKAAPKKAAPAPKPKVEDGIFGTSGGIGFTKQNELFVGRVAMIGFAASLLGEAITGKGILSQLNLETGIPIYEAEPLLLFFILFTLLGAIGALGDRGRFVDEPTGLDKAVIPPGKGFRSALGLSEGGPLFGFTKANELFVGRLAQLGIAFSLIGEIITGKGALAQLNIETGLPISEIEPLVLFNVAFFFVAALNPGTGKFVTDEEED

SEQ ID NO: 94 - protein, 271 aa, Phalaenopsis equestris
MAQSMLMSSFGGHLVDCNRDPLLRTQIRRLRPSPLSHLFLSRPSPSQSTSPSSYAPPLAVFKSKTKAPSRKVEKDRPKVEDGIFGTSGGIGFTKANELFVGRVAMLGFAASLIGEGITGKGILAQLNLETGIPIYEAEPLLLFFILFTLLGAIGALGDRGSFVDDPPTGLDKAVIAPGKGFRSALGLKEGGPLFGFTKANELFVGRLAQLGIVFSLIGEIITGKGALAQLNIETGVPLNELEPLVLFNVVFFFFAALNPGSGKFVTDEDVE

SEQ ID NO: 95 - protein, 273 aa, Musa acuminata
  REGION 1..273: note = subsp. Malaccensis
MAQSVLLASTGGRSLSSTMEPLLQAQIHRLRPTRFSDLILPRTPRRHLPATSSSSFFPTLALFKAKTKAPPAKVETKKPKVEDGIFGTSGGIGFTKQNELFVGRVAMLGFAASLLGEGITGKGILAQLNLETGIPIYEAEPLLLFFILFTLLGAIGALGDRGQFVDDSPTGLGKAVIPPGKGFRAALGLEEGGPLFGFTKANELFVGRLAQLGIAFSIIGEIITGKGALAQLNIETGVPVSELEPLVLFNVAFFFFAALNPGTGKFVTDEVEE

SEQ ID NO: 96 - protein, 273 aa, Cinnamomum micranthum f. kanehirae
MAQSMLMSSVSGQLVPSRTEALLQFQIRRLCPTPFSHILLSPSTHQRPPSPSTSISTPTLALFKSKTKAPPAKKVVEKPKVEDGIFGTSGGIGFTKQNELFVGRVAMIGFAASLLGEGITGKGILAQLNLETGIPIYEAEPLLLFFILFTLLGAIGALGDRGRFVDDPPTGLERAVIPPGKSIRSALGLKEGGPLFGFTKSNELFVGRLAQLGIAFSIIGEIITGKGALAQLNIETGVPLQDIEPLVLFNVIFFFFAALNPGNGKFVTDEEDK

SEQ ID NO: 97 - protein, 274 aa, Phoenix dactylifera
MVHAVFTSGLGAHLPDSKREPLLQSQLRRLRPTPFSHLLLSQPSHKQLPSPSYSSYTPVLALFKSKTKAPPKKVEKAKPKVEDGIFGTSGGIGFTKQNELFVGRVAMIGFAASLLGEAITGKGILAQLNLETGIPIYEAEPLLLFFILFTLLGAIGALGDRGRFVDDPPTGLDKAVIPPGKGIRSALGLKEGGPLFGFTKSNELFVGRLAQLGIAFSIIGEIVTGKGALAQLNIETGVPINEIEPLVLFNVIFFFIAALNPGNGKFVTDVDEEE

SEQ ID NO: 98 - protein, 273 aa, Elaeis guineensis
MAQAVFTSRLGSQLLDSKTEPLLQSQLRRLRPTPFSHLLLSQPSHKQIPSPSYSSYTPVLALFKSKTKAPPKKVEKTKPKIEDGIFGTSGGIGFTKQNELFVGRVAMIGFAASLLGEAITGKGILAQLNLETGIPIYEAEPLLLFFILFTLLGAIGALGDRGRFVDDPSTGLGKAVIPPGKGFRSALGLKEGGPLFGFTKANELFVGRLAQLGIAFSIIGEIITGKGALAQLNIETGVPINEIEPLVLFNVIFFFFAALNPGTGKFLTDEDEE

SEQ ID NO: 99 - protein, 279 aa, Punica granatum
MAQAMLVMPSASGHSVDVKRDGLLRFQIQRLRPKSSFPSLLGFNPLLTPASLDSRRPVSSPAVALFKSKTKAPPKKVAKEPPKPKVEDGIFGTSGGIGFTKENELFVGRVAMLGFAASLLGEGITGKGILAQLNLETGIPIYEAEPLLLFFILFTLLGAIGALGDRGKFVDDPATGIEGAVIPPGKSFRAAVGLKEGGPLFGFTKANELFVGRLAQLGFAFSLIGEIITGKGALAQLNVETGVPINEIEPLVLFNVLFFFIAAINPGTGKFVTDEDQEN

SEQ ID NO: 100 - protein, 272 aa, Carica papaya
MAQTMLLMHSTHAALDLKKQSLLHTQLRIKPFSPLFFSPLPPSSSSSSSSSSKPFTTLALFKSKTKAAPAKKVEKPKLKTEDGIFGTSGGIGFTKQNELFVGRVAMLGFAASLLGEAITGKGILAQLNLETGIPIYEAEPLLLFFILFTLLGAIGALGDRGRFVDDPPTGLEGAVIRPGKGIRGALGLNEGGPLFGFTKANELFVGRLAQLGIAFSLIGEIITGKGALAQLNFETGIPISDIEPIVLFNVAFFFFAAINPGTGKFITDEDEE

SEQ ID NO: 101 - protein, 278 aa, Corchorus capsularis
MAQTMLLMSSTSVSTGHAVLNLKKEHPLFQLQAHGLKPKPFSHFLFNPLSTNTVSTSSKGFTTFAIFKPRTKAAPKKAAPKPKLQVEDGIFGTSGGIGFTKQNELFVGRVAMIGFAASLLGEAITGKGILAQLNLETGIPIYEAEPLLLFFILFTLLGAIGALGDRGRFVDDPPTGIEGAVIPPGKGIRGALGLKEGGPLFGFTKSNELFVGRLAQLGIAFSLIGEIITGKGALAQLNIETGIPVNEIEPLVLLNVVFFFLAAINPGTGKFVTDEEDD

SEQ ID NO: 102 - protein, 278 aa, Corchorus olitorius
MAQTMLLMSSTSVSTGHAVLNLKKEHPLFQLQAHGLKPKPFSRFLFNPLSTNTVSTSSKGFTTFAIFKPRTKAAPKKAAPKPKLQVEDGIFGTSGGIGFTKQNELFVGRVAMIGFAASLLGEAITGKGILAQLNLETGIPIYEAEPLLLFFILFTLLGAIGALGDRGRFVDDPPTGIEGAVIPPGKGIRGALGLKEGGPLFGFTKSNELFVGRLAQLGIAFSLIGEIITGKGALAQLNIETGIPVNEIEPLVLLNVVFFFLAAINPGTGKFVTDEEED

SEQ ID NO: 103 - protein, 269 aa, Gossypium arboreum
MAQTMLLMSSHAVSLKRDHPLLHFQAQGLKPKPVSHLFFNPLSNGVHTSSKAFTTLAMFKSKTKAAPKRVEPKSKPKVEDGIFGTSGGIGFTKQNELFVGRVAMLGFAASLLGEAITGKGILAQLNLETGIPIYEAEPLLLFFILFTLLGAIGALGDRGRFVDDPPTGIEGAVIPPGKGIRGALGLKEGGPLFGFTKANELFVGRLAQLGIAFSLIGEIITGKGALAQLNIETGIPISDIEPLVLFNVAFFFFAAINPGTGKFLTDEEE

SEQ ID NO: 104 - protein, 269 aa, Gossypium arboreum
MAQTMLLMSSHAVSLKRDHPLLHFQAQGLKPKPVSHLFFNPLSNGVHTSSKAFTTLAIFKSKTKAAPKRVEPKSKPKVEDGIFGTSGGIGFTKQNELFVGRVAMLGFAASLLGEAITGKGILAQLNLETGIPIYEAEPLLLFFILFTLLGAIGALGDRGRFVDDPPTGIEGAVIPPGKGIRGALGLKEGGPLFGFTKANELFVGRLAQLGIAFSLIGEIITGKGALAQLNIETGIPISDIEPLVLFNVAFFFFAAINPGTGKFLTDEEE

SEQ ID NO: 105 - protein, 268 aa, Gossypium hirsutum
MAQTMLLMSSHAVSLKRDHPLLHFQAQGLKPKPASHLFFNPLSNGVHTSPKAFTTLAVFKSKTKAAPKRVEPKPKVEDGIFGTSGGIGFTKQNELFVGRVAMLGFAASLLGEAITGKGILAQLNLETGIPIYEAEPLLLFFILFTLLGAIGALGDRGRFVDDPPTGIEGAVIPPGKGIRGALGLKEGGPLFGFTKANELFVGRLAQLGIAFSLIGEIITGKGALAQLNIETGIPISDIEPLVLFNVAFFFFAAINPGTGKFLTDEEEE

SEQ ID NO: 106 - protein, 276 aa, Theobroma cacao
MAQTMLLMSSTSVSTSHVVNLKRGHPLLQFQAQGLKPKPFFHFLLNPFSDSVATSSRGFTTFAVFKPKTKAAPKRAAAPPKLKVEDGIFGTSGGIGFTKQNELFVGRVAMLGFAASLLGEAITGKGILAQLNLETGIPIYEAEPLLLFFILFTLLGAIGALGDRGSFVDDPPSGIEGAVIPPGRGIRGALGLKEGGPLFGFTKANELFVGRLAQLGIAFSLIGEIITGKGALAQLNIETGIPISDIEPLVLFNVAFFFFAAINPGTGKFVTDEDEK

SEQ ID NO: 107 - protein, 276 aa, Theobroma cacao
MAQTMLLMSSTSVSTSHVVNLKRGHPLLQFQAQGLKPKPFFHFLLNPFSDSVATSSRGFTTFAVFKPKTKAAPKRVAAPPKLKVEDGIFGTSGGIGFTKQNELFVGRVAMLGFAASLLGEAITGKGILAQLNLETGIPIYEAEPLLLFFILFTLLGAIGALGDRGSFVDDPPSGIEGAVIPPGRGIRGALGLKEGGPLFGFTKANELFVGRLAQLGIAFSLIGEIITGKGALAQLNIETGIPISDIEPLVLFNVAFFFFAAINPGTGKFVTDEDEE

SEQ ID NO: 108 - protein, 277 aa, Gossypioides kirkii
MAQTMLVMSSTSVSASHVVNLKRDHPLLHFQAQGLKPKPFSHFLFNPLSNAVAASSSKAFTTFALFKSKTKAAPKKAEPKPKPKVEDGIFGTSGGIGFTKQNELFVGRVAMIGFAASLLGEAITGKGILAQLNLETGIPIYEAEPLLLFFILFTLLGAIGALGDRGRFVDEPPTGVEGAVIPPGKGVRGALGLKEGGPLFGFTKANELFVGRLAQLGIAFSLIGEIITGKGALAQLNIETGIPISDIEPLVLFNVAFFFAAAINPGTGKFVTDEEED

SEQ ID NO: 109 - protein, 278 aa, Gossypium hirsutum
MAQTMLVMSSTSVSTSHVVNLKRDHPLLHFQAQGLKPKPSSHFLFNPHSNTVAASSSKAFTTFALFKPKTKAAPKKAAPKPKPKVEDGIFGTSGGIGFTKQNELFVGRVAMIGFAASLLGEAITGKGILAQLNLETGIPIYEAEPLLLFFILFTLLGAIGALGDRGKFVDEPPTGIEGAVIPPGKGIRGTLGLKEGGPLFGFTKANELFVGRLAQLGIAISLIGEIITGKGALAQLNIETGIPISDIEPLALFNVAFFFFAAINPGTGKFVTDEAEED

SEQ ID NO: 110 - protein, 278 aa, Gossypium arboreum
MAQTMLVMSSTSVSTSHVVNLKRDHPLLHFQAQGLKPKPSSHFLFNPHSNTVAASSSKAFTTFALFKPKTKAAPKKAAPKPKPKVEDGIFGTSGGIGFTKQNELFVGRVAMIGFAASLLGEAITGKGILAQLNLETGIPIYEAEPLLLFFILFTLLGAIGALGDRGKFVDEPPTGIEGAVIPPGKGIRGALGLKEGGPLFGFTKANELFVGRLAQLGIAFSLIGEIITGKGALAQLNIETGIPISDIEPLVLLNVAFFFFAAINPGTGKFVTDEAEED

SEQ ID NO: 111 - protein, 274 aa, Gossypium hirsutum
MLVMSSTSVSTSHVVNLKRDHPLLHFQAQGLKPKPSSHFLFNPHSNTVAASSSKAFTTFALFKPKTKAAPKKAAPKPKPKVEDGIFGTSGGIGFTKQNELFVGRVAMIGFAASLLGEAITGKGILAQLNLETGIPIYEAEPLLLFFILFTLLGAIGALGDRGKFVDEPPTGIEGAVIPPGKGIRGALGLKEGGPLFGFTKANELFVGRLAQLGIAFSLIGEIITGKGALAQLNIETGIPISDIEPLVLFNVAFFFFAAINPGTGKFVTDEAEED

SEQ ID NO: 112 - protein, 278 aa, Gossypium hirsutum
MAQTMLVMSSTSVSTSHVVNLKRDHPLLHFQAQGLKPKPSSHFLFNPHSNTVAASSSKAFTTFALFKPKTKAAPKKAAPKPKPKVEDGIFGTSGGIGFTKQNELFVGRVAMIGFAASLLGEAITGKGILAQLNLETGIPIYEAEPLLLFFILFTLLGAIGALGDRGKFVDEPPTGIEGAVIPPGKGIRGALGLKEGGPLFGFTKANELFVGRLAQLGIAFSLIGEIITGKGALAQLNIETGIPISDIEPLVLFNVAFFFFAAINPGTGKFVTDEAEED

SEQ ID NO: 113 - protein, 278 aa, Gossypium hirsutum
MAQTMLVMSSTRVSTSHVVNLKRDHTLLHFQAQGLKPKPSSHFLFNPHSNTVAASSSKAFTTFALFKPKTKAAPKKAAPKPKPKVEDGIFGTSGGIGFTKQNELFVGRVAMIGFAASLLGEAITGKGILAQLNLETGIPIYEAEPLLLFFILFTLLGAIGALGDRGKFVDEPPTGIEGAVIPPGKGIRGALGLKEGGPLFGFTKANELFVGRLAQLGIAFSLIGEIITGKGALAQLNIETGIPISDIEPLVLFNVAFFFFAAINPGTGKFVTDEAEED

SEQ ID NO: 114 - protein, 278 aa, Gossypium raimondii
MAQTMLVMSSTSVSTSHVVNLKRDHTLLHFQAQGLKPKPSSHFLFNPHSNTVAASSSKAFTTFALFKPKTKAAPKKAAPKPKPKVEDGIFGTSGGIGFTKQNELFVGRVAMIGFAASLLGEAITGKGILAQLNLETGIPIYEAEPLLLFFILFTLLGAIGALGDRGKFVDEPPTGIEGAVIPPGKGIRGALGLKEGGPLFGFTKANELFVGRLAQLGIAFSLIGEIITGKGALAQLNIETGIPISDIEPLVLFNVAFFFFAAINPGTGKFVTDEAEED

SEQ ID NO: 115 - protein, 274 aa, Durio zibethinus
MAQTVLLMSSTSVSTSHVVNLKRNQPLLHFQPQGLKPKPFSHFLFNPPSNGVATSSKAFTTFAIFKSKTKAVPKKVAQKPKVEDGIFGTSGGIGFTKQNELFVGRVAMLGFAASLLGEAITGKGILAQLNLETGIPIYEAEPLLLFFILFTLLGAIGALGDRGRFVDDPPTGIEGAVIPPGKGIRGALGLKEGGPLFGFTKSNELFVGRLAQLGIVFSLIGEIITGKGALAQLNIETGIPINDIEPLVLFNVAFFFFAAINPGTGKFLTDEDEE

SEQ ID NO: 116 - protein, 276 aa, Durio zibethinus
MAQTMLLMSSTCVSTSHVVNLKRDLPLLHFQAQGLKPKPFSHFLFNPLSNGVATSSKAFTTFAVFKSRTKAAPKKVEPKPKPKVEDGIFGTSGGIGFTKQNELFVGRVAMLGFAASLLGEAITGKGILAQLNLETGIPIYEAEPLLLFFILFTLLGAIGALGDRGSFVDDPPTGVEGAVIPPGKGIRGALGLKEGGPLFGFTKANELFVGRLAQLGIAFSLIGEIITGKGALAQLNIETGIPINDIEPLVLFNVAFFFFAAINPGTGKFVTDEDEE

SEQ ID NO: 117 - protein, 273 aa, Nelumbo nucifera
MAQSMMLMSRVAGHVLDLKREPVLQSQVQRLRPKPFSHLVLPPLSTRPHSTSSSASTVVSLFKARTKAAPAKVEKPKEKVEDGIFGTSGGIGFTKQNELFVGRVAMLGFAASLLGEGITGKGILAQLNLETGIPIYEAEPLLLFFILFTLLGAIGALGDRGRFVDDPPTGLERAVIPPGKSFRAALGLNEGGPLFGFTKANELFVGRLAQLGIAFSLIGEIITGKGALAQLNIETGVPINELEPLLLFNVAFFFFAAINPGTGKFLTDEEEDE

SEQ ID NO: 118 - protein, 270 aa, Vitis vinifera
MAQTMLITPSVSGHSVLDLKRQPLLQRLRPKPFSHLLLPPLPTSSSTSSPAFTTLALFKSKTKAAPSKKVTKPKPQVEDGIFGTSGGIGFTKKNELFVGRVAMLGFAASLLGEAITGKGILAQLNLETGIPIYEAEPLLLFFILFTLLGAIGALGDRGRFVDDDPPTGIEGAVIPPGKGLRSALGLREGGPLFGFTKSNELFVGRLAQLGIVFSIIGEIITGKGALAQLNIETGVPISDIEPLVLFNVIFFFFAALNPGSGKFVTDDEEE

SEQ ID NO: 119 - protein, 270 aa, Vitis vinifera
MAQTMLITPSVSGHSVLDLKRQPLLQRLRPKPFSHLLLPPLPTSSSTSSPAFTTLALFKSKTKAAPPKKVTKPKPQVEDGIFGTSGGIGFTKKNELFVGRVAMLGFAASLLGEAITGKGILAQLNLETGIPIYEAEPLLLFFILFTLLGAIGALGDRGRFVDDDPPTGIEGAVIPPGKGLRSALGLREGGPLFGFTKSNELFVGRLAQLGIVFSIIGEIITGKGALAQLNIETGVPISDIEPLVLFNVIFFFFAALNPGSGKFVTDDEEE

SEQ ID NO: 120 - protein, 281 aa, Citrus sinensis
MAQTMLLMSGVSTSHAGNLKRDSLVTFQTQMLRPKPFSHLMFNPLSNESLTAAAASSSSKAFSTVALFKSKTKAPPKTKKVESKPKVEDGIFGTSGGFGFTKQNELFVGRVAMIGFAASLLGEGITGKGILAQLNLETGIPIYEAEPLLLFFILFTLLGAIGALGDRGKFVDEPDEPTGLGGAVIPPGKGLRSALGLKEGGPIFGFTKSNELFVGRLAQLGIAFSLIGEIITGKGALAQLNIETGIPINEIEPLVLFNVIFFFVAALNPGTGKFVTDEDEE

SEQ ID NO: 121 - protein, 281 aa, Citrus clementina
MAQTMLLMSGVSTSHAGNLKRDSLVTFQTQMLRPKPFSHLMFNPLSNESLTAAAASSSSKAFSTVALFKSKTKAPPKTKKVESKPKVEDGIFGTSGGFGFTKQNELFVGRVAMIGFAASLLGEGITGKGILAQLNLETGIPIYEAEPLLLFFILFTLLGAIGALGDRGKFVDEPDEPTGLGGAVIPPGKSLRSALGLKEGGPIFGFTKSNELFVGRLAQLGIAFSLIGEIITGKGALAQLNIETGIPINEIEPLVLFNVIFFFVAALNPGTGKFVTDEDEE

SEQ ID NO: 122 - protein, 274 aa, Cephalotus follicularis
MAQTMLLMCGISTSHVVDLRRDPLFHVQIQKLRPKSFSHLFFNPLSNNGFSLAQKFNTLALFKSKTKAAPKKVATTAKPKVEDGLFGTSGGIGFTKQNELFVGRVAMIGFAASLLGEAVTGKGILSQLNLETGIPIYEAEPLLLFFILFTLLGAIGALGDRGRFVDDEPTGLERAVIPPGKGIRGALGLREGGPLFGFTKSNELFVGRLAQLGIAFSLIGEIITGKGALAQLNIETGIPINEIEPLVLFNVIFFFFAALNPGTGKFVTDEEAED

SEQ ID NO: 123 - protein, 273 aa, Juglans regia
MAQTILLMSGVSSSHVVDLKRDALLNFQLQKLRPKPFSHLLLPHLPANTTSSSPVFTTFALFKAKTKAPPKKVAPKPKEKVEDGIFGTSGGIGFTKQNELFVGRVAMIGFAASLLGEGITGKGILSQLNLETGIPIYEAEPLLLFFILFTLLGAIGALGDRGKFVDDDTTGLEKAVIPPGKGLRSALGLKEGGPLFGFTKSNELFVGRLAQLGIAFSLIGEIITGKGALAQLNIETGIPINEIEPLVLFNVIFFFFAALNPGTGKFVTDEEDN

SEQ ID NO: 124 - protein, 273 aa, Populus tomentosa
MAQTMVLMSGVSTRQVVDLKRDPLLQFQVQRLRPAPFSRLLYNPLPSKASSSNAFTTLALFKPRTKAVPKKAAPPPKPKVEDGIFGTSGGIGFTKQNELFVGRVAMIGFAASLLGEAITGKGILSQLNLETGIPIYEAEPLLLFFILFTLLGAIGALGDRGRFVDDPPTGIEGAVIPPGKSLRAALGLKEGGSPLFGFTKSNELFVGRLAQLGIAFSLIGEIITGKGALAQLNIETGIPVSEIEPLVLFNVLFFFVAALNPGTGKFVTDEDEE

SEQ ID NO: 125 - protein, 272 aa, Populus trichocarpa
MAQTMVLMSGVSTRQVVDLKRDPLLQFQVQRLRPAPFSRLLYNPLPSKASSSNAFTTLALFKPRTKAVPKKAAPPPKPKVEDGIFGTSGGIGFTKQNELFVGRVAMIGFAASLLGEAITGKGILSQLNLETGIPIYEAEPLLLFFILFTLLGAIGALGDRGRFVDDPTTGIEGAVIPPGKSFRAALGLKEGGPLFGFTKSNELFVGRLAQLGIAFSLIGEIITGKGALAQLNIETGIPVNEIEPLVLFNVLFFFIAALNPGTGKFVTDEDEE

SEQ ID NO: 126 - protein, 272 aa, Populus euphratica
MAQTMVLMSGVPTRQVVDLKRDPLLQFQVQRLRPAPFSRLLYNPLPSKASSSNAFTTLALFKPRTKAVPKKAAPPPKPKVEDGIFGTSGGIGFTKQNELFVGRVAMIGFAASLLGEAITGKGILSQLNLETGIPIYEAEPLLLFFILFTLLGAIGALGDRGRFIDDPTTGIEGAVIPPGKSFRAALGLKEGGPLFGFTKSNELFVGRLAQLGIAFSLIGEIITGKGALAQLNIETGIPVNEIEPLVLFNVLFFFVAALNPGTGKFVTDEDEE

SEQ ID NO: 127 - protein, 275 aa, Hevea brasiliensis
MAQTMLLMSGVSTRHVVHFKRDPLLQFQVERLRPKPFSHLLLSRLSANYTCFSSRAFSTLALFKSKTKAPPKKAAVKPKPKVEDGIFGTSGGIGFTKQNELFVGRVAMLGFAASLLGEAITGKGILAQLNLETGIPIYEAEPLLLFFILFTLLGAIGALGDRGKFVDDTSTGLEGAVIPPGKSFRTALGLKEGGPLFGFTKANELFVGRLAQLGIAFSLIGEIITGKGALAQLNIETGIPINEIEPLVLFNVVFFFIAAVNPGNGKFVTDEDEDD

SEQ ID NO: 128 - protein, 275 aa, Ricinus communis
MAQTMLFMSGVSTRHVVDLKRDPLLQFQVDRLRPKPFSSLLFSPLPTNSSSFAPSKTFTTFALFKSKAKAAPKKTVVKPKQKVEDGVFGTSGGIGFTKQNELFVGRVAMLGFAASLLGEGLTGKGILAQLNLETGIPIYEAEPLLLFFILFTLLGAIGALGDRGKFVDDPPTGIEGAVIPPGKGFRAALGLKEGGPLFGFTKSNELFVGRLAQLGIAFSLIGEIITGKGALAQLNIETGIPVNEIEPLVLFNVIFFFFAALNPGTGKFVTDEEDE

SEQ ID NO: 129 - protein, 274 aa, Jatropha curcas
MAQTMLFISGVSTRHVVDLKRDPLLQFQVERLRPKPFSNLVFNPISISKTSFSTSKPFTTLALFKSKAKAPPKKVEKPKEKVEDGIFGTSGGIGFTKQNELFVGRVAMLGFAASLLGEAVTGKGILAQLNLETGIPIYEAEPLLLFFILFTLLGAIGALGDRGKFVDDPPTGIEGAVIPPGKSFRAALGLKEGGPLFGFTKANELFVGRLAQLGIAFSLIGEIITGKGALAQLNIETGIPINELEPLVLFNIIFFFIAALNPGTGKFVTDEEED

SEQ ID NO: 130 - protein, 279 aa, Physcomitrella patens
MAQAALISRIGAAQALGGVAQSQTLSTRRLSSLFAASSSSTLPVGLGLNRNAERVSSGAVKTFALFGKTKPAAKAPAPTKGKAKVEDGIFGTSGGIGFTKANELFVGRVAMLGFAASILGEALTGKGTLAQFDIETGIPLTETEPLLLFFILFTLLGAIGALGDRGKFVDDAPVAGLDSTIIKPGKGVKGALGLNEKGPVFGFTKSNELFVGRLAQLGIAFAIIGEIITGKGALAQLNIETGVPITELEPLILFNVIFFLFAAVNPGTGKFVNDDDIED

SEQ ID NO: 131 - protein, 282 aa, Marchantia polymorpha
MAQATMLARSLATVALSRNVQVSESSLKVQPFSARQLSSLHGSSVSSFAAGSASQVGVARSGKVFALFKSAKKEAPKKQDKKPVKSSTEDGIFGTSGGIGFTKANELFVGRVAMLGFSASILGEALTGKGTLAQFDVETGIPLNETEPLLLFFILFTLLGAIGALGDRGKFVDDDVDEANTGGVIPPGKGFKAALGLNQKGPTFGFTKANELFVGRLAQLGIAFSIIGEIITGKGTLAQLNIETGLPITELEPLILFNVIFFFLAAINPGTGKFINDDDIEE

SEQ ID NO: 132 - protein, 281 aa, Marchantia polymorpha
  REGION 1..281: note = subsp. Ruderalis
MAQATMLARSLATVALSRNVQVSESSLKVQPFSARQLSSLHGSSVSSFAAGSASQVGVARSGKVFALFKSAKKEAPKKDKKPVKSSTEDGIFGTSGGIGFTKANELFVGRVAMLGFSASILGEALTGKGTLAQFDVETGIPLNETEPLLLFFILFTLLGAIGALGDRGKFVDDDVDEANTGGVIPPGKGFKAALGLNQKGPTFGFTKANELFVGRLAQLGIAFSIIGEIITGKGTLAQLNIETGLPITELEPLILFNVIFFFLAAINPGTGKFINDDDIEE

SEQ ID NO: 133 - protein, 266 aa, Selaginella moellendorffii
MAQAALLSRVTTALNGIAQQRLSSTRSNAIGGSSQQLLGQRLGVVVTPQHFSSASRIVRTFALKSKAPATSKKSKSTVEDGIFGTSGGIGFTKQNELFVGRVAMIGFAASLLGEAITGKGVLAQFDLETGIPLNETEPLLLFFILFTLLGAIGALGDRGKFVDETPGPIIEPGKGFKSAIGLKEKGPVFGFTKSNELFVGRLAQLGIAFSIIGELITGKGALAQLNIETGVPITEIEPLLLFNIIFFFIAAINPGTGRFIADNEEE

SEQ ID NO: 134 - protein, 266 aa, Selaginella moellendorffii
MAQAALLSRVTTALNGIAQQRLSSTRSNAIGGSSQQLLGQRLGVVVAPQHFSSASRIVRTFALKSKAPATSKKSKSTVEDGIFGTSGGIGFTKQNELFVGRVAMIGFAASLLGEAITGKGVLAQFDLETGIPLNETEPLLLFFILFTLLGAIGALGDRGKFVDETPGPIIEPGKGFKSAIGLKEKGPVFGFTKSNELFVGRLAQLGIAFSIIGELITGKGALAQLNIETGVPITEIEPLLLFNIIFFFIAAINPGTGRFIADNEEE

SEQ ID NO: 135 - protein, 294 aa, Picea sitchensis
MAQSVLPSRSVRSYVGGIAAFSQRPLPAPPALPVARGLRIRPPNAAETSWSPLRELPSANAAGLNARTANTVALFRPKEKAAAPPKKGRTGRVSVPEEKPKVEDGIFGTSGGIGFTKANELFVGRVAMIGFAASLLGEAITGKGILAQLNLETGIPIYEAEPLLLFFILFTLLGAIGALGDRGKFVDEAPTPDSLVDPGKGFRSAVGLKDSGPLFGFTKSNELFVGRLAQLGIAFSIIGEIITGKGALAQLNIETGVPVSEIEPLVLFNVAFFLFAALNPGNGKFVTDVDEEEE

SEQ ID NO: 136 - protein, 297 aa, Picea sitchensis
MAQSVLASGGLSSCVAGIAGFNQRPLPSPPALPVARGLRIRPPNAAQISWSPLTELPSANAAGLNARIARTEALFRPKEKAAAPPKKGRTGRVALPEEKPKVEDGIFGTSGGIGFTKANELFVGRVAMIGFAASLLGEAITGKGILSQLNLETGIPIYEAEPLLLFFILFTLLGAIGALGDRGKFVDDQTPTTGLEKAVIPPGKGFRSAVGLNESGPLFGFTKSNELFVGRLAQLGIAFSIIGEIITGKGALAQLNIETGVPVSEIEPLVLFNVAFFLFAALNPGNGKFVTDEEEEE

SEQ ID NO: 137 - protein, 271 aa, Zea mays
MAQSMITSSVLGCLSSSTVRKDTLLQFQIQRLRPTSSSTLFLSPKSSPGRSTQSTLFFQPLALFKSKAKTIPKKVVVKPKVESGIFGTSGGIGFTKENELFVGRVAMLGFAASLLGEGLTGKGILSQLNLETGIPIYEAEPLLLFFILFTLLGAIGALGDRGRFEDEATGLDKAVIAPGKNLRTSFGLKEGGPLFGFTKANELFVGRMAQLGIAFSIIGEIITGKGALAQLNIETGVPITEIEPLVLFNVVFFFIAALNPGSGKFKTDDEE

SEQ ID NO: 138 - protein, 273 aa, Sedum alfredii
MAQSTLLMFNGALPTFKTHSSLQSQIQRLVKPNNNPFLQTPLLHSKASSPLGLGFTTVALFKAKAKAPAKKETKSKPKLKVEDGVFGTSGGIGFTKQNELFVGRVAMIGFAASILGEAVTGKGILAQLNLETGIPIYEAEPLLLFFILFTLLGAIGALGDRGRFVDDPPTGLEKAVIPPGKGLRSAFGLSEGGPLFGFTKSNELFVGRLAQLGFAFSLIGEIITGKGALAQLNIETGVPINEIEPLVLFNVIFFFFAALNPGTGKFVTDDEED

SEQ ID NO: 139 - protein, 268 aa, Dorcoceras hygrometricum
MAAQTTMLISTGNRVSGHEPLVRGLKPNPFPRFLFPSIPTVDRPASSSSATTRYVIESMAKAPPAVKKAPAVKKKSTVEDGIFGTSGGIGFTKQNELFVGRVAMIGFAASLLGEAITGKGILAQLNLETGIPIYEAEPLLLFFILFNLLGAIGALGDRGRFVDDATGLDKTVVAPGKGLRAALGLGQGGRLFGFTKANELFVGRLAQLGIAFSIIGEIITGKGALAQLNIETGVPINEIEPLLLFNIVFFFVAAINPGSGKFVTDDEE

SEQ ID NO: 140 - protein, 271 aa, Erythranthe guttata
MAQTMLLNASNVSSVVRTTTNEPLLQRLKPKSFSQFLLTPRTTSATSTPAFSSTLALFKPKTKAPPPKKAPVVKEKSKVEDGIFGTSGGIGFTKQNELFVGRVAMIGFAASLLGEALTGKGILAQLNLETGIPIYEAEPLLLFFILFNLLGAIGALGDRGRFVDEPTTGLDKAVIAPGKGFRTAIGLGEGGPLFGFTKSNELFVGRLAQLGIAFSIIGEIVTGKGALAQLNIETGVPISEIEPLLLFNVAFFFFAAINPGTGKFVTDDGDE

SEQ ID NO: 141 - protein, 270 aa, Erythranthe guttata
MAAQTMLLTGSSSHVVKGETNTLLIQKLKPMPLSQFLFPQRTNHSSFSSSSASIVSLFKPKTKAPPKKAAPVVKEKSKVEDGIFGTSGGIGFTKQNELFVGRVAMIGFAASLLGEALTGKGILAQLNLETGIPIYEAEPLLLFFILFTLLGAIGALGDRGRFVDEEPAGLGKAVIAPGKGFRAALGLGEGGPLLGFTKSNELFVGRLAQLGIAFSIIGEIITGKGGLAQLNIETGVPINELEPLLLFNVAFFFFAAINPGSGKFVTDDED

SEQ ID NO: 142 - protein, 265 aa, Cynara cardunculus
  REGION 1..265: note = var. scolymus
MAQTMLLTSNTVVSGHPLLQSLRPKPFSHLLLPPSLVNASSTSPARTYTSPVALFKAKTKAPAKKAAPKEKIKVEDGIFGTSGGIGFTKQNELFVGRVAMIGFAASLLGEGLTGKGILQQLNLETGIPIYEAEPLLLFFILFTLLGAIGALGDRGRFVDEPTGLDKAVIPPGKGLRSALGLSEGGPLFGFTKSNELFVGRLAQLGIAFSLIGEIITGKGALAQLNIETGIPISDIEPLVLFNVAFFFVAALNPGTGKFVTDDEED

SEQ ID NO: 143 - protein, 262 aa, Helianthus annuus
MAQTMLLTSNTIISDQPLLRSLRPKPFSHRVLAPGLLPNSSTTFLSPVALFKPKTKAAPKKAVTPPPKLKVEDGIFGTSGGIGFTKQNELFVGRVAMLGFAASLLGEGITGKGILSQLNLETGIPIYEAEPLLLFFILFTLLGAIGALGDRGRFVDEPTGLDKAIIPPGKGFRSALGLSEGGPLFGFTKANELFVGRLAQLGIAFSLIGEIITGKGALAQLNIETGLPVSEIEPLVLFNVAFFFIAALNPGTGKFVTDEEED

SEQ ID NO: 144 - protein, 264 aa, Dorcoceras hygrometricum
MAAQTMLLTANAINAGGEALIRRLKPKPFSHFLLPSKAGSAPSSACGIVAIFKAKPKADPVKKGPAAKEKLKVEDGIFGTSGGIGFTKQNELFVGRVAMIGFAASLLGEAITGKGILAQLNLETGIPIYEAEPLLLFFILFNLLGAIGALGDRGRFVDDTTGLDKAVISPGKGFRSALGLGEGGPLFGFTKANELFVGRLAQLGIAFSIIGEIITGKGALAQLNFETGVPITEIEPLILFNVAFFFIAAINPGTGKFVTDDGDD

SEQ ID NO: 145 - protein, 269 aa, Olea europaea
  REGION 1..269: note = var. sylvestris
MAQTMLLSAGSTVSGHALDLKKDPLMMQRLKPKPFSHFVLPPKTSSPLSSSTTTVALFKSKTKAPAKKVPVVKEKPKVEDGIFGTSGGFGFTKENELFVGRVAMIGFAASLLGEAVTGKGILAQLNLETGIPLYEAEPLLLFFILFNLLGAIGALGDRGRFVDDPPTGLDKAVIAPGKGIRGALGLGDGPLFGFTKSNELFVGRLAQLGIVFSIIGEIVTGKGALAQLNIETGVPINELEPLLLFNIVFFFIAAINPGTGKFVTDEEEA

SEQ ID NO: 146 - protein, 269 aa, Olea europaea
  REGION 1..269: note = var. sylvestris
MVQTMLFSASSTVSGHALDLKKEQLMIQRLKPKPFSHFLLPPKTSSPLSSSTTTVALFKSKAKAPAKKIAVVKEKTKVEDGIFGTSGGFGFTKENELFVGRVAMIGFAASLLGEAVTGKGILSQLNLETGIPIYEAEPLLLFFILFNLLGAIGALGDRGRFVDETPTGLDKAVIAPGKGFRGALGLGDGPLFGFTKSNELFVGRLAQLGIVFSIIGEIVTGKGALAQLNIETGVPINELEPLLLFNIVFFFIAAINPGTGKFVTDEEEA

SEQ ID NO: 147 - protein, 260 aa, Handroanthus impetiginosus
MAQTMLLTANVSGEPLLIQKLRKPFAHQFLLPKRSFSRFSTSTTTYAFFKSKTKAAPPKKAVVVKEKSKVEDGIFGTSGGIGFTKQNELFVGRVAMIGFAASLLGEAITGKGILAQLNLETGIPIYEAEPLLLFFILFNLLGAIGALGDRGRFVDEPAGLEKAVIPPGKGFRAALGLGEGPLFGFTKANELFVGRLAQLGIAFSIIGEIITGKGALAQLNFETGVPINEIEPLLLFNILFFFVAAINPGTGKFVTDEEED

SEQ ID NO: 148 - protein, 262 aa, Plantago major
MAQTMLLSANVSAHLKSEQPLIKTFKPKAFSNFVLPPRTNVSRSLPFTTYALFKSKTKAPTKAPVVKEKPKVEDGIFGTSGGIGFTKQNELFVGRVAMIGFAASLLGEAITGKGILAQLNLETGIPIFEAEPLLLFFILFNLLGAIGALGDRGRFVDEPTGLDKAVIAPGKGFRAALGLGSGPLFGFTKANELFVGRLAQLGIAFSIIGEIVTGKGALAQLNVETGVPIGELEPLLLFNIVFFFVAAINPGTGKFVTDEEED

SEQ ID NO: 149 - protein, 264 aa, Sesamum indicum
MAQTMLLTANVSGHVKGEQLLIHTLKPKPFSHFLLPPRTRTTSSSHLPSSTTVALFKSKTKAPPAKKVVEKPKVEDGIFGTSGGIGFTKQNELFVGRVAMIGFAASLLGEAITGKGILAQLNLETGIPIYEAEPLLLFFILFNLLGAIGALGDRGRFVDEPTGLEKAVIPPGKGFRAALGLGDGPLFGFTKANELFVGRLAQLGIAFSIIGEIVTGKGALAQLNIETGVPINEIEPLLLFNILFFFVAAINPGTGKFVTDEEED

SEQ ID NO: 150 - protein, 271 aa, Arabidopsis thaliana
MPQSLLMSGAAIDFKRDNALLQFQSTHSLLRPAPFSHSVRPPLPPRRCLVLGTPFPLALFKSKTKAAPVKKVAESKPKVEDGIFGTSGGIGFTKQNELFVGRVAMIGFAASILGEAITGKGILAQLNLETGIPIYEAEPLLLFFILFTLLGAIGALGDRGRFVDDSPTGLDKAVIPPGKGIRSALGLPEGGPLFGFTKANELFVGRLAQLGIAFSIIGEIVTGKGALAQLNIETGVPLSDIEPLVLFNVLFFFVAALNPGSGKFVTDEDKD

SEQ ID NO: 151 - protein, 272 aa, Musa acuminata
  REGION 1..272: note = subsp. Malaccensis
MAQSMLVSSIGGKEAALLQSQTHRLRPTPFSHLLLPRVPHGRQLPPPAASFFPTLAIFKPKTKAPAKKQVAKPKPKRKVEDGIFGTSGGIGFTKQNELFVGRVAMIGFAASILGEAITGKGILAQLNLETGIPIYEAEPLLLFFILFTLLGAIGALGDRGSFVDDPPTGLEGAVIPPGKGFRSALGLKEGGPLFGFTKANELFVGRLAQLGIAFSIIGEIVTGKGALAQLNIETGVPISDIEPLVLFNVVFFFFAALNPGTGKFVTDVEEEE

SEQ ID NO: 152 - protein, 274 aa, Ensete ventricosum
MMAQSMLVSSIGGKEAALLQSQTHRLRPTPFSHLLLPRVPHGRQLPPPAASFFPTLAIFKPRTKAPAKKQVASFPKPKQKVEDGIFGTSGGIGFTKQNELFVGRVAMLGFAASILGEAITGKGILAQLNLETGIPIYEAEPLLLFFILFTLLGAIGALGDRGSFVDDPPTGLEGAVIPPGKGFRSALGLKEGGPLFGFTKANELFVGRLAQLGIAFSIIGEIVTGKGALAQLNIETGVPIGEIEPLVLFNVLFFFFAALNPGTGKFVTDVEEEE

SEQ ID NO: 153 - protein, 274 aa, Ensete ventricosum
MMAQSMLVSSIGGKEAALLQSQTHRLRPTPFSHLLLPRVPHGRQLPPPAASFFPTLAIFKPRTKAPAKKQVASFPKPKQKVEDGIFGTSGGIGFTKQNELFVGRVAMLGFAASILGEAITGKGILAQLNLETGIPIYEAEPLLLFFILFTLLGAIGALGDRGSFVDDPPTGLEGAVIPPGKGFRSALGLKDGGPLFGFTKSNELFVGRLAQLGIAFSIIGEIVTGKGALAQLNIETGVPISEIEPLVLFNVLFFFFAALNPGTGKFVTDVEEEE

SEQ ID NO: 154 - protein, 268 aa, Sorghum bicolor
MAQSMLMSTSVNGGRALPSLQAGRPAPYPRLPLPSSSSGYRHSKSVSVKTLALFGKSKVKTAPSKKAAAPKPKPKVEDGIFGTSGGIGFTKENELFVGRVAMLGFAASLLGEAITGKGILAQLNLETGIPIYEAEPLLLFFILFTLLGAIGALGDRGRFVDEEVTGLDKAVIQPGKGFRGALGLSEGGPLFGFTKSNELFVGRLAQLGVAFSIIGEIITGKGALAQLNIETGVPINEIEPLVIFNVLFFFVAAINPGNGRFIIGEDEE

SEQ ID NO: 155 - protein, 268 aa, Saccharum hybrid
  REGION 1..268: note = cultivar R570
MAQSMLMSTSVNGGRALPSLQAGRPAPYPRLPQPSSSSGYRHSKSVSVKTLALFGKSKVKTAPSKKAAAPKPKPKVEDGIFGTSGGIGFTKENELFVGRVAMLGFAASLLGEAITGKGILAQLNLETGIPIYEAEPLLLFFILFTLLGAIGALGDRGRFVDEEVTGLDKAVIQPGKGFRGALGLSEGGPLFGFTKSNELFVGRLAQLGVAFSIIGEIITGKGALAQLNIETGVPINEIEPLVIFNVLFFFVAAINPGNGRFIIGEDEE

SEQ ID NO: 156 - protein, 265 aa, Dichanthelium oligosanthes
MAQSMIMSTSVSGGRALPSLQAVRPAPPYPRLPLPSVYRHSKSVSVKTLALFGGKSKAAKAAPVKKAPAPKPKVEDGIFGTSGGIGFTKENELFVGRVAMLGFAASLLGEAITGKGILAQLNLETGIPIYEAEPLLLFFILFTLLGAIGALGDRGTFVDDVTGLDKAVIQPGKGFRGALGLSEGGPLFGFTKSNELFVGRLAQLGVAFSIIGEIITGKGALAQLNIETGVPINEIEPLVLFNVLFFFIAAVNPGTGKFIISEEEE

SEQ ID NO: 157 - protein, 265 aa, Setaria italica
MAQSMLMSTSVSGGRALPSLQAARPAAAYPRLALPSVNRHSKSVSVKTLALFGKSKAAKAAPAKKVAAPKPKVEDGIFGTSGGIGFTKENELFVGRVAMLGFAASLLGEAITGKGILAQLNLETGIPIYEAEPLLLFFILFTLLGAIGALGDRGTFVDDVTGLDKAVIQPGKGFRGALGLSEGGPLFGFTKSNELFVGRLAQLGVAFSIIGEIITGKGALAQLNIETGVPINEIEPLVLFNVLFFFIAAINPGTGKFIIGDDEKE

SEQ ID NO: 158 - protein, 262 aa, Panicum miliaceum
MAQSMLMSTSVSGGRALPSLQAGRPAPYLRLALPSAYRHSRSVSVKTLALFGKSKAAKAPAKKAPAPKPKVEDGIFGTSGGIGFTKENELFVGRVAMLGFAASLLGEAITGKGILAQLNLETGIPIYEAEPLLLFFILFTLLGAIGALGDRGTFVDDVTGLDKAVIPPGKGFRGALGLSEGGPLFGFTKSNELFVGRLAQLGVAFSIIGEIITGKGALAQLNIETGVPINEIEPLVLFNVLFFFIAAINPGTGKFIIGEDEE

SEQ ID NO: 159 - protein, 262 aa, Panicum hallii
MARSMLMSTSVSGGRALPSLQAGRPAPYLRLALPSAYRHSRSVSVKTLALFGKSKAAKAPAKKAPAPKPKVEDGIFGTSGGIGFTKENELFVGRVAMLGFAASLLGEAITGKGILAQLNLETGIPIYEAEPLLLFFILFTLLGAIGALGDRGTFVDDVTGLDKAVIPPGKGFRGALGLSEGGPLFGFTKSNELFVGRLAQLGVAFSIIGEIITGKGALAQLNIETGVPINEIEPLVLFNVLFFFIAAINPGTGKFIIGEDEE

SEQ ID NO: 160 - protein, 269 aa, Brachypodium distachyon
MAQSMLMSSVNGVASSGRSLLQAARPAATPFSRLALPASPSYYKHMPSLSVRTMAIFGKSKAKAAPAKKVAAPKPKTEDGIFGTSGGIGFTKENELFVGRVAMIGFAASILGEAITGKGILSQLNLETGIPIYEAEPLLLFFILFTLLGAIGALGDRGKFVDEQPTGLDKAVIAPGKGFRSALGLSEGGPLFGFTKSNELFVGRLAQLGIAFSIIGEIITGKGALAQLNIETGVPISEIEPLVLFNVVFFFVAAINPGTGKFISGEEDD

SEQ ID NO: 161 - protein, 271 aa, Mycobacteroides abscessus
MAQSMLMSGVNGVASGRSLLQAARPSSASTPFSRLALSSSSAAYYKHMPSLSVRTMALFGKSKTKAAPAKKVVAPKPKTEDGIFGTSGGIGFTKENELFVGRVAMIGFAASILGEAITGKGILSQLNLETGIPIYEAEPLLLFFILFTLLGAIGALGDRGRFVDEQPTGLDKAVIAPGKGFRSALGLSEGGPLFGFTKSNELFVGRLAQLGIAFSIIGEIITGKGALAQLNIETGVPISEIEPLVLFNVVFFFIAAINPGTGKFISGEDDD

SEQ ID NO: 162 - protein, 272 aa, Triticum aestivum
MAQSMLMSGVNGVASGRSLLQAARPSTASTPFSRLALSSSSAAYYKHMPSLSVRTMALFGKSKTKAAPAKKVAAPKPKTEDGIFGTSGGIGFTKENELFVGRVAMIGFAASILGEAITGKGILSQLNLETGIPIYEAEPLLLFFILFTLLGAIGALGDRGRFVDEQPTTGLDKAVIAPGKGFRSALGLSEGGPLFGFTKSNELFVGRLAQLGIAFSIIGEIITGKGALAQLNIETGVPISEIEPLVLFNVVFFFIAAINPGTGKFISGEEDD

SEQ ID NO: 163 - protein, 273 aa, Aegilops tauschii
  REGION 1..273: note = subsp. Tauschii
MAQSMLMSGVNGVASGRSLLQAARPSSASTPFSRLALSSSSSAAAYYKHMPSLSVRTMALFGKKTKAAPAKKVVAPKPKTEDGIFGTSGGIGFTKENELFVGRVAMIGFAASILGEAITGKGILSQLNLETGIPIYEAEPLLLFFILFTLLGAIGALGDRGRFVDEQPTTGLDKAVIAPGKGFRSALGLSEGGPLFGFTKSNELFVGRLAQLGIAFSIIGEIITGKGALAQLNIETGVPISEIEPLVLFNVVFFFIAAINPGTGKFISGEEDD

SEQ ID NO: 164 - protein, 271 aa, Triticum urartu
MAQSMLMSGVNGVASGRSLLQAARPSSASTPFSRLALSSSSSAAYYKHMPSLSVRTMALFGKKTKAAPAKKVAAPKPKTEDGIFGTSGGIGFTKENELFVGRVAMIGFAASILGEAITGKGILSQLNLETGIPIYEAEPLLLFFILFTLLGAIGALGDRGRFVDEQPTGLDKAVIAPGKGFRSALGLSEGGPLFGFTKSNELFVGRLAQLGIAFSIIGEIITGKGALAQLNIETGVPISEIEPLVLFNVVFFFIAAINPGTGKFISGEEDD

SEQ ID NO: 165 - protein, 269 aa, Phyllostachys edulis
MARSMLMSSVSGVASASGRSLLHAVRPTPLSRFVLSSQPSYCKRMAPLSVKTVALFGGKSKAKAAPARKAETKPKFKTEDGIFGTSGGIGFTKENELFVGRVAMLGFAASILGEAITGKGILAQLNLETGIPIYEAEPLLLFFILFTLLGAIGALGDRGSFVDEPTGLDKAIVPPGKGLRSALGLGEGGPLFGFTKANELFVGRLAQLGIAFSIIGEIITGKGALAQLNIETGVPISEIEPLVLFNVIFFFVAAINPGTGKFVTTDDEE

SEQ ID NO: 166 - protein, 250 aa, Oryza brachyantha
MYMYMQQAMPMVVCGRPSWGEKQMMMRGRRRQVMVVVAAFKSRSKAAPAPAKRQKVAVEDGIFGTSGGIGFTKENELFVGRVAMLGFAASLLGEAVTGKGILAQLNLETGIPIYEAEPLLLFFILFTLLGAIGALGDRGRFVDDATGIERAVIPPGKGFRAALGLREGGPLFGFTKANELFVGRLAQLGIAFSLIGEIITGKGALAQLNIETGVPVNEIEPLLLFSILFFFFAAINPGTGKFVTDNDQDQ

SEQ ID NO: 167 - protein, 270 aa, Apostasia shenzhenica
MAQSMLISSFAAGQLVGCNNSRVGAAPSLSKLFLLENSSSRRRQPSSSASPHAPTLAVFKPKTKAPVKKIEKTSKPKVEDGIFGTSGGIGFTKQNELFVGRVAMLGFAASLLGEGLTGKGILAQLNLETGIPIYEAEPLLLFFILFTVLGAIGALGDRGKFVDDPPAGLDKAVIPPGKGLRSALGLKEGGPLFGFTKANELFVGRLAQLGIAFSIIGEIITGKGALAQLNIETGVPINEVEPLVIFNVIFFFLAALNPGSGRFVTDEGEE

SEQ ID NO: 168 - protein, 271 aa, Tarenaya hassleriana
MAQTMLLTSGVSAGSLLNHNRNPLSGPELRPRHGLFSSADSPALPSRRLSSPALVPLAIFKPKTKAAPAKKAPPPKPKVEDGIFGTSGGIGFTKQNELFVGRVAMIGFAASLLGEAVTGKGILAQLNLETGIPIYEAEPLLLFFILFTLLGAIGALGDRGQFVDDPPTGLDKAVIPPGKSFRSAIGLPEQGPLFGFTKANELFVGRLAQLGIAFSLIGEIITGKGALAQLNFETGIPITEIEPIVLFNVAFFFFAAINPGTGKFITDDEED

SEQ ID NO: 169 - protein, 271 aa, Coffea arabica
MAQAMLLTASVPSSHAGDLKGSEPLLQRIRPKPISHLFFSPLPTSSSSSYCPLTTVAVFKSKTKAPAKKPAAATKPKEKVEDGIFGTSGGIGFTKENELFVGRVAMLGFAASILGEAITGKGILAQLNLETGIPIYEAEPLLLFFILFNLLGAIGALGDRGRFVDDAPGIEGAVVPPGKGLRGALGLREGGPLFGFTKANELFVGRLAQLGIAFSIIGEIITGKGALAQLNIETGVPITEIEPLIIFNVVFFFVAAINPGTGKFVTDEEDA

SEQ ID NO: 170 - protein, 271 aa, Coffea arabica
MAQAMLLTASVPSSHAGDLKRSEPLLQRIRPKPISHLFFSPLPTSSSSSYCPLTTVAVFKSKTKAPAKKPAAATKPKEKVEDGIFGTSGGIGFTKENELFVGRVAMLGFAASILGEAITGKGILAQLNLETGIPIYEAEPLLLFFILFNLLGAIGALGDRGRFVDDAPGIEGAVVPPGKGLRSALGLREGGPLFGFTKANELFVGRLAQLGIAFSIIGEIITGKGALAQLNIETGVPITEIEPLIIFNVVFFFVAAINPGTGKFVTDEEDA

SEQ ID NO: 171 - protein, 271 aa, Coffea canephora
MAQAMLLTASVPSSHAGDLKRSEPLLQRIRPKPISHLFFSPLPTSSSSSYCPLTTVAVFKSKTKAPAKKPAAATKPKEKVEDGIFGTSGGIGFTKENELFVGRVAMLGFAASILGEAITGKGILAQLNLETGIPIYEAEPLLLFFILFNLLGAIGALGDRGRFVDDAPGIEGAVVPPGKGLRGALGLREGGPLFGFTKANELFVGRLAQLGIAFSIIGEIITGKGALAQLNIETGVPITEIEPLIIFNVVFFFVAAINPGTGKFVTDEEDA

SEQ ID NO: 172 - protein, 269 aa, Ananas comosus
MAQSMLLSSLSGHPLASKRDPLLQSLRPTPFSHLIFSQTPRKELSPSSSSSFTPTLAVFKSKAKAPPKKLEKDAKAKVEDGIFGTSGGIGFTKQNELFVGRVAMIGFAASLLGEAITGKGILAQLNLETGIPIYEAEPLLLFFILFTLLGAIGALGDRGQFVDETAGLEKAVIPPGKGFKSALGLKEGGPLFGFTKANELFVGRLAQLGIAFSLIGEIITGKGALAQLNIETGVPINEIEPLLLFNIAFFFFAAINPGTGKFITDEEEA

SEQ ID NO: 173 - protein, 281 aa, Diospyros kaki
MAAQAMLLTSSTVDLKRQQLPLIERLRPKPYFSHFLLPPLSSSSTSSPSSSSSSSSSSVHYNSSPTIALFKSKAKAAPPKKVATPKPKVEDGLFGTSGGIGFTKQNELFVGRVAMLGFAASLLGEAITGKGILAQLNLETGIPIYEAEPLLLFFILFTLLGAIGALGDRGKFVDESPTGLDKAVIPPGKGFRSSLGLKEGGPLFGFTKANELFVGRLAQLGIAFSIIGEIITGKGALAQLNIETGVPISDIEPLVLFNVAFFFIAALNPGTGKFVTDEEEE

SEQ ID NO: 174 - protein, 274 aa, Ipomoea nil
MAQTMLLTASASSPALDLKRQSTFLQTLKPKPSIQQHFLLPLPSSSSSSSSFSSTRIVALFKSKAKAAPAKKVVQPKPKVEDGIFGTSGGIGFTKQNELFVGRVAMIGFAASLLGEAITGKGILSQLNLETGIPIYEAEPLLLFFILFNLLGAIGALGDRGKFVDDPAPPTGLDRAVIPPGKSFRSALGLSEGGPLFGFTKSNELFVGRLAQLGIAFSIIGEIITGKGALAQLNIETGVPIGEIEPLILFNVAFFFFAAINPGTGKFVTDEEED

SEQ ID NO: 175 - protein, 272 aa, Nicotiana tomentosiformis
MAQTMLLTANAKVDLRSKESLVERLKPKPLSSFFLPSLPLKYPSASSSSHFASTTVALFKSKAKAPAKKAVPKPKEKVEDGIFGTSGGIGFTKQNELFVGRVAMIGFAASLLGEAVTGKGILAQLNLETGIPIYEAEPLLLFFILFNLLGAIGALGDRGKFIDDPAPATGLDKAVIPPGKGFKAALGLREGGPLFGFTKANELFVGRLAQLGIAFSIIGEIITGKGALAQLNFETGVPINEIEPLLLFNIVFFFIAAINPGTGKFVTDEEEE

SEQ ID NO: 176 - protein, 272 aa, Nicotiana attenuata
MAQTMLLTANAKVDLRSKESLVERLKPKPLSSFFLPSLPLKYPSASSSSHFTSTTVALFKSKAKAPAKKVVPKPKEKVEDGIFGTSGGIGFTKQNELFVGRVAMIGFAASLLGEAVTGKGILAQLNLETGIPIYEAEPLLLFFILFNLLGAIGALGDRGKFVDDPAPATGLDKAVIPPGKGFKAALGLREGGPLFGFTKANELFVGRLAQLGIAFSIIGEIITGKGALAQLNFETGVPINEIEPLLLFNIVFFFIAAINPGTGKFVTDEEEE

SEQ ID NO: 177 - protein, 274 aa, Nicotiana sylvestris
MAQTMLLTANAKVDLRSKESLVERLKPKPLSSFFLPSLPLKYPSASASASSHFTSTTVALFKSKAKAPAKKVVPKPKEKVEDGIFGTSGGIGFTKQNELFVGRVAMIGFAASLLGEAITGKGILAQLNLETGIPIYEAEPLLLFFILFNLLGAIGALGDRGKFIDDPAPPTGLDKAVIPPGKGFKSALGLSEGGPLFGFTKANELFVGRLAQLGIAFSIIGEIITGKGALAQLNFETGVPINEIEPLLLFNIVFFFVAAINPGTGKFVTDEEEE

SEQ ID NO: 178 - protein, 277 aa, Capsicum annuum
MAQTMLLTANAKVDLRSKGSLVERLKPKPLSSLFLPSLPLKFSTTATNASSSKFTSTTVAMFKSKAKAAPAKKAAPKPKEKVEDGIFGTSGGIGFTKQNELFVGRVAMIGFAASLLGEAITGKGILAQLNLETGIPIYEAEPLLLFFILFNLLGAIGALGDRGKFVDDPAPPTGLEKAVIPPGRSFKSALGLSEGGPLFGFTKANELFVGRLAQLGIAFSIIGEIITGKGALAQLNIETGVPINEIEPLLLFNVAFFFFAAINPGTGKFITDGEEED

SEQ ID NO: 179 - protein, 277 aa, Capsicum baccatum
MAQTMLLTANAKVDLRSKGSLVERLKPKPLSSLFLPSLPLKFSTTATNASSSKSTSTTVAMFKSKAKAAPAKKAAPKPKEKVEDGIFGTSGGIGFTKQNELFVGRVAMIGFAASLLGEAITGKGILAQLNLETGIPIYEAEPLLLFFILFNLLGAIGALGDRGKFVDDPAPSTGLEKAVIPPGRSFKSALGLSEGGPLFGFTKANELFVGRLAQLGIAFSIIGEIITGKGALAQLNIETGVPINEIEPLLLFNVAFFFFAAINPGTGKFITDGEEED

SEQ ID NO: 180 - protein, 276 aa, Solanum sogarandinum
MAQTMLLTANAKVDLRSKESLVERLKPKPLSSLFLPSLPLRFSSSTTNFSSSKFTSTTVALFKSKAKAPPKKVAPPKEKQKVEDGIFGTSGGIGFTKQNELFVGRVAMIGFAASLLGEAITGKGILAQLNLETGIPIYEAEPLLLFFILFNLLGAIGALGDRGRFIDDPAPATGLEKAVIPPGKSFKSALGLSEGGPLFGFTKANELFVGRLAQLGIAFSIIGEIITGKGALAQLNFETGVPINEIEPLLLFNIAFFFFAAINPGTGKFITDEEED

SEQ ID NO: 181 - protein, 276 aa, Solanum tuberosum
MAQTMLLTANAKVDLRSKESLVERLKPKPLSSLFLPSLPLRFSSSTTNVSSSKFTSTTVALFKSKAKAPPKKVAPPKEKQKVEDGIFGTSGGIGFTKQNELFVGRVAMIGFAASLLGEAITGKGILAQLNLETGIPIYEAEPLLLFFILFNLLGAIGALGDRGKFIDDPSPATGLEKAVIPPGKSFKSALGLTEGGPLFGFTKANELFVGRLAQLGIAFSIIGEIITGKGALAQLNFETGVPINEIEPLLLFNIAFFFFAAINPGTGKFITDEEED

SEQ ID NO: 182 - protein, 276 aa, Solanum nigrum
MAQTMLLTANAKVDLRSKESLVERLKPKPLSSLFLPSLPLRFSSSSTNASSSKFTSTTVALFKSKAKAPPKKVAPPKQKEKVEDGIFGTSGGIGFTKQNELFVGRVAMIGFAASLLGEAITGKGILAQLNLETGIPIYEAEPLLLFFILFNLLGAIGALGDRGKFVDDPAPPTGLEKAVIPPGRSFKSALGLSEGGPLFGFTKANELFVGRLAQLGIAFSIIGEIITGKGALAQLNFETGVPINEIEPLLLFNIAFFFFAAINPGTGKFITDEEED

SEQ ID NO: 183 - protein, 276 aa, Solanum pennellii
MAQTMLLTANAKVDLRSKESLVERLKPKPLSSLFLPSLPLRFSSSTTNVSSSKFTSTTVALFKSKAKAPPKKVAPPKEKQKVEDGIFGTSGGIGFTKQNELFVGRVAMIGFAASLLGEAITGKGILAQLNLETGIPIYEAEPLLLFFILFNLLGAIGALGDRGKFVDDPTPPTGLEKAVIPPGKSFKSALGLSEGGPLFGFTKANELFVGRLAQLGIAFSIIGEIITGKGALAQLNFETGVPINEIEPLLLFNIAFFFFAAINPGTGKFITDEEED

SEQ ID NO: 184 - protein, 276 aa, Solanum lycopersicum
MAQTMLLTANAKVDLRSKESLVERLKPKPLSSLFLPSLPLRFSSSSTNASSSKFTSTTVALFKSKAKAPPKKVAPPKEKQKVEDGIFGTSGGIGFTKQNELFVGRVAMIGFAASLLGEAITGKGILAQLNLETGIPIYEAEPLLLFFILFNLLGAIGALGDRGKFVDDPTPPTGLEKAVIPPGKSFKSALGLSEGGPLFGFTKANELFVGRLAQLGIAFSIIGEIITGKGALAQLNFETGVPINEIEPLLLFNIAFFFFAAINPGTGKFITDEEED

(19) United States
(12) Patent Application Publication
(10) Pub. No.: US 2024/0417746 A1
(43) Pub. Date: Dec. 19, 2024

(54) PLANTS WITH INCREASED WATER USE EFFICIENCY

(71) Applicants: The Board of Trustees of the University of Illinois, Urbana, IL (US); The Regents of the University of California, Oakland, CA (US)

(72) Inventors: Stephen P. Long, Champaign, IL (US); Katarzyna Glowacka, Urbana, IL (US); Johannes Kromdijk, Cambridge (GB); Krishna K. Niyogi, Oakland, CA (US); Laurie Beth Leonelli, Oakland, CA (US)

(21) Appl. No.: 18/755,523

(22) Filed: Jun. 26, 2024

Related U.S. Application Data
(62) Division of application No. 16/808,881, filed on Mar. 4, 2020, now Pat. No. 12,054,727.
(60) Provisional application No. 62/814,005, filed on Mar. 5, 2019.

(51) Int. Cl. C12N 15/82

(52) U.S. Cl. CPC C12N 15/8273

(57) ABSTRACT

Aspects of the present disclosure relate to methods of cultivating genetically altered plants with increased activity of the PsbS protein. These genetically altered plants have increased water use efficiency and substantially similar photosynthetic efficiency as compared to WT plants grown under the same conditions.

### CROSS-REFERENCE TO RELATED APPLICATION

This application is a divisional application of and claims the benefit of priority to U.S. patent application Ser. No. 16/808,881, entitled “Plants with Increased Water Use Efficiency,” filed Mar. 4, 2020, which claims the benefit of priority to U.S. Provisional Application No. 62/814,005, entitled “Plants with Increased Water Use Efficiency,” filed Mar. 5, 2019, the disclosures of which are hereby incorporated by reference in their entireties.

### INCORPORATION BY REFERENCE OF SEQUENCE LISTING PROVIDED AS A TEXT FILE

This application contains references to nucleic acid sequences that have been submitted concurrently herewith as the sequence listing text file “BERK-500DIV_ST26.xml”, file size 236,967 bytes, created on Jun. 25, 2024, which is incorporated by reference in its entirety pursuant to 37 C.F.R. § 1.52(e)(5).

### TECHNICAL FIELD

The present disclosure relates to genetically altered plants. In particular, the present disclosure relates to genetically altered plants with increased water use efficiency.

### BACKGROUND

Water use efficiency (WUE) in plants is becoming increasingly important and is especially important in arid and semiarid regions for environmentally sustainable food production. Food crop production in such regions requires significant supplies of water. Improving WUE in crops will be needed to ensure that these regions can transition to and maintain such environmentally sustainable food production. As time passes the need will only increase. Climate change predictions show increasing temperatures and even drought in these regions and more areas becoming semiarid. Thus, with time, food crops with improved WUE will become a necessity for meeting global food production demands in light of increasing populations and climate change. Equally, food crops with improved WUE will decrease the amount of water required in irrigated agriculture and improve the yield stability of rain fed crops in drier years.

One significant area for improving WUE in crops is through reducing transpiration. Plants lose upwards of 98-99% of the water they absorb via transpiration through their stomata. Stomatal opening and closing impacts both water vapor efflux and CO₂ influx in the leaf. If the stomata remain open to allow for maximal CO₂ capture, the plant will lose water. Conversely, if the stomata remain closed to reduce stomatal conductance and conserve water, CO₂ influx is reduced and the rate of photosynthesis will decline. Therefore, stomatal opening and closing is regulated by a variety of environmental cues, and these environmental cues are perceived and integrated within the plant in order to optimally balance water vapor efflux and CO₂ influx.

Progress has been made in unraveling the molecular mechanisms underlying stomatal response to the environmental cues of CO₂ concentration and blue light. However, the molecular mechanisms underlying stomatal response to red light remain elusive. Even though individual components relating to stomatal response have been identified, it is not yet clear how the diverse signals regulating stomatal response are integrated in the plant and translated into signals regulating stomata. Given the potential tradeoffs in reducing transpiration and the risk of adversely impacting yields through reduced photosynthesis, stomatal regulation of WUE remains a challenging problem.

A related area of research into improving crops involves a similarly complex issue affecting photosynthesis: dynamic and fluctuating light intensity, especially high light intensity or rapidly increasing light intensity. Over time, this can damage the photosynthetic apparatus, which can result in photoinhibition or persistent reduction of photosynthetic yield. In order to avoid this, plants have evolved photoprotective mechanisms, which are induced when excess light energy is present. One photoprotective mechanism, non-photochemical quenching of chlorophyll fluorescence (NPQ), harmlessly dissipates the excess light energy as heat. NPQ changes, while fast, still lag behind light intensity changes, i.e. changes in absorbed light energy. This lag increases with prolonged or repeated high light conditions, meaning that under these conditions, the plant is increasingly exposed to potential photodamage. While the NPQ quenching sites and mechanisms are still being elucidated, research has demonstrated that photosystem II subunits as well as multiple components of the carotenoid biosynthesis pathway are important for NPQ processes. Therefore, developing plants with reduced NPQ lag that can be used to engineer crops with improved photosynthetic efficiency and therefore higher yields requires multi-component engineering.

Photosystem II Subunit S (PsbS) is a primary component of the photosynthetic apparatus, which is conserved across all plants. When PsbS is overexpressed in conjunction with two other proteins important for NPQ processes, namely zeaxanthin epoxidase (ZEP) and violaxanthin de-epoxidase (VDE), improves the rate of NPQ changes. This multi-component engineering was found to accelerate recovery of NPQ on sun to shade transitions, in turn allowing faster recovery of photosynthetic efficiency and improved crop productivity (Kromdijk et al., Science 2016).

### BRIEF SUMMARY

In order to test whether the improvement in photosynthetic efficiency and crop productivity observed when modifying three components of the NPQ pathway could be achieved through modification of a single component, PsbS overexpression was evaluated on its own. PsbS overexpression alone did not improve the photosynthetic efficiency. However, surprisingly, it was found that PsbS overexpression resulted in plants with reduced stomatal conductance and increased water use efficiency. This surprising new utility for PsbS identified by the inventors serves as the basis for many of the aspects and their various embodiments of the present disclosure.

An aspect of the disclosure includes a method of cultivating a genetically altered plant with increased water use efficiency, including the steps of: (a) providing the genetically altered plant, wherein the plant or a part thereof includes one or more genetic alterations; and (b) cultivating the genetically altered plant under conditions wherein the one or more genetic alterations increase activity of a Photosystem II Subunit S (PsbS) protein as compared to a wild type (WT) plant without the one or more genetic alterations, and wherein the increased activity of the PsbS protein increases water use efficiency as compared to the WT plant grown under the same conditions. An additional embodiment of this aspect includes the conditions being reduced irrigation conditions. A further embodiment of this aspect includes the conditions being rain fed conditions. Yet another embodiment of this aspect includes the conditions being high density growth conditions. Still another embodiment of this aspect includes the conditions being mild salinity. In an additional embodiment of this aspect, the conditions are fertilized or providing additional nutrients. In a further embodiment of this aspect, the conditions are humid conditions or conditions resulting in wet leaf surfaces. In another embodiment of this present aspect, which may be combined with any of the preceding embodiments, the increased activity of the PsbS protein provides the genetically altered plant with a higher yield, an increased biomass, an increased growth rate, an increased tolerance of salinity, an increased ability to withstand salinity, an increased flow of nutrients to the roots, an increased availability of nutrients over time, an increased utilization of fertilizer, an increased utilization of nutrients, a decreased susceptibility to a plant disease requiring humid conditions and/or wet leaf surfaces for infection, a decreased susceptibility to infection by the plant disease, a reduced incidence of the plant disease, or a reduced incidence of infection by the plant disease as compared to the WT plant without the increased activity grown under the same conditions where the activity of the PsbS protein is increased.

In yet another embodiment of this present aspect, which may be combined with any of the preceding embodiments, the genetically altered plant does not include increased activity of zeaxanthin epoxidase (ZEP) protein, violaxanthin de-epoxidase (VDE) protein, or both, as compared to a WT plant. In a further embodiment of this present aspect, which may be combined with any of the preceding embodiments, the genetically altered plant does not include reduced activity of K+efflux antiporter 3 (KEA3) as compared to a WT plant.

In still another embodiment of this aspect that can be combined with any of the relevant preceding embodiments, the genetically altered plant further includes increased activity of a ZEP protein and increased activity of a VDE protein, wherein the increased activity of the PsbS protein is greater than the increased activity of the PsbS protein required for increased photosynthetic efficiency when each of the ZEP protein, the PsbS protein, and the VDE protein are overexpressed together. An additional embodiment of this aspect includes increased activity of the PsbS protein being due to an increased amount of the PsbS protein. In yet another embodiment of this aspect, the increased amount of the PsbS protein is at least 10% greater, at least 20% greater, at least 30% greater, at least 40% greater, at least 50% greater, at least 75% greater, at least 100% greater, at least 150% greater, or at least 200% greater than the increased activity of the PsbS protein required for increased photosynthetic efficiency when each of the ZEP protein, the PsbS protein, and the VDE protein are overexpressed together. Still another embodiment of this aspect includes the increased amount of the PsbS protein being no greater than 500%, no greater than 400%, no greater than 300%, no greater than 200%, no greater than 150%, no greater than 100%, no greater than 75%, or no greater than 50%, of the increased activity of the PsbS protein required for increased photosynthetic efficiency when each of the ZEP protein, the PsbS protein, and the VDE protein are overexpressed together. Another embodiment of this aspect includes the increased amount of PsbS, the increased amount of ZEP, and the increased amount of VDE being increased relative to an amount of endogenous PsbS protein, an amount of endogenous ZEP protein, and an amount of endogenous VDE protein, and wherein the increased amount of PsbS is 10% or more, 12% or more, 14% or more, 16% or more, 18% or more, 20% or more, 22% or more, 24% or more, 30% or more, 40% or more, 50% or more, 100% or more, or 200% or more increased relative to the amount of endogenous PsbS protein, the increased amount of ZEP is 90% increased relative to the amount of endogenous ZEP protein, and the increased amount of VDE is 80% increased relative to the amount of endogenous VDE protein. In a further embodiment of this aspect, the increased amount of PsbS, the increased amount of ZEP, and the increased amount of VDE is increased relative to an amount of endogenous PsbS protein, an amount of endogenous ZEP protein, and an amount of endogenous VDE protein, and wherein the increased amount of PsbS is 12% increased relative to the amount of endogenous PsbS protein, the increased amount of ZEP is 90% increased relative to the amount of endogenous ZEP protein, and the increased amount of VDE is 80% increased relative to the amount of endogenous VDE protein. In yet another embodiment of this aspect, which may be combined with any of the preceding embodiments that has increased activity of a ZEP protein and increased activity of a VDE protein, the increased activity of the PsbS, VDE, and ZEP proteins provides the genetically altered plant with increased photosynthetic efficiency under non-steady state conditions as compared to the WT plant without the increased activity grown under the same conditions where the activity of the PsbS, VDE, and ZEP proteins is increased.

Still another embodiment of this present aspect, which may be combined with any of the preceding embodiments, includes the genetically altered plant being selected from the group of cowpea, soybean, cassava, wheat, barley, corn, sorghum, rice, cotton, sugarcane, eucalyptus, poplar, willow, orange, grapefruit, lemon, lime, avocado, cherry, peach, plum, apricot, nectarine, fig, olive, almond, pistachio, walnut, chestnut, hazelnut, pecan, tomato, eggplant, potato, or alfalfa. An additional embodiment of this aspect includes the genetically altered plant not being rice or Arabidopsis.

A further embodiment of this aspect that can be combined with any of the preceding embodiments includes the genetically altered plant being provided in step (a) by planting a genetically altered seedling, a genetically altered plantlet, a genetically altered cutting, a genetically altered tuber, a genetically altered root, or a genetically altered seed to produce the genetically altered plant, or by grafting the genetically altered seedling, the genetically altered plantlet, or the genetically altered cutting to a root stock or a second plant to produce the genetically altered plant. In still another embodiment of this aspect that can be combined with any of the preceding embodiments, the genetically altered plant is cultivated in step (b) to produce harvestable seed and fruits or vegetatively produced harvested items. An additional embodiment of this aspect includes the harvestable seed and fruits being selected from seed, fruit, pods, grain, kernels, beans, and peas; and wherein the vegetatively produced harvested items are selected from tubers, rhizomes, buds, roots, cuttings, and leaves. A further embodiment of this aspect that can be combined with any of the preceding embodiments that has harvestable seed and fruit or vegetatively produced harvestable items further includes harvesting the harvestable seed, harvestable leaves, harvestable roots, harvestable cuttings, harvestable fruit, harvestable kernels, harvestable tubers, harvestable pods, harvestable peas, harvestable beans, and/or harvestable grain.

Still another embodiment of this aspect that can be combined with any of the preceding embodiments includes the increased activity of the PsbS protein providing the genetically altered plant with decreased stomatal conductance as compared to the WT plant without the increased activity grown under the same conditions where the activity of the PsbS protein is increased. In yet another embodiment of this aspect, which may be combined with any of the preceding embodiments that does not have increased activity of a ZEP protein and increased activity of a VDE protein, the increased activity of the PsbS protein provides the genetically altered plant with substantially similar photosynthetic efficiency as compared to the WT plant grown under the same conditions where the activity of the PsbS protein is increased.

In a further embodiment of this aspect that can be combined with any of the preceding embodiments, increased activity is increased expression. An additional embodiment of this aspect includes the increased expression being due to expression of a heterologous PsbS protein. A further embodiment of this aspect includes the heterologous PsbS protein including at least 70% sequence identity to, at least 75% sequence identity to, at least 80% sequence identity to, at least 85% sequence identity to, at least 90% sequence identity to, at least 95% sequence identity to, or at least 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184. Yet another embodiment of this aspect includes the heterologous PsbS protein being selected from the group of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184. A further embodiment of this aspect that can be combined with any of the preceding embodiments that has a heterologous PsbS protein includes the heterologous PsbS protein containing a glutamate at a position corresponding to amino acid 149 of reference sequence SEQ ID NO: 21 and a glutamate at a position corresponding to amino acid 255 of reference sequence SEQ ID NO: 21. Still another embodiment of this aspect that can be combined with any of the preceding embodiments that has a heterologous PsbS protein includes the heterologous PsbS protein being encoded by a first nucleic acid and the first nucleic acid being operably linked to a second nucleic acid including a promoter. An additional embodiment of this aspect includes the promoter being selected from the group of a CaMV35S promoter, a ubiquitin promoter, a Rbcs1A promoter, a GAPA-1 promoter, a FBA2 promoter, or any combination thereof. Still another embodiment of this aspect that can be combined with any of the preceding embodiments that has a nucleic acid sequence includes the first nucleic acid sequence and the second nucleic acid sequence being stably integrated into a nuclear genome of the plant or into a chloroplast genome of the plant. A further embodiment of this aspect includes the increased expression being due to overexpression of an endogenous PsbS protein. An additional embodiment of this aspect includes overexpression of the endogenous PsbS protein being achieved using a gene editing technique to introduce the one or more genetic alterations that increase the activity of the endogenous PsbS protein. Still another embodiment of this aspect includes the gene editing technique being selected from the group of transcription activator-like effector nuclease (TALEN) gene editing techniques, clustered Regularly Interspaced Short Palindromic Repeat (CRISPR/Cas) gene editing techniques, or zinc-finger nuclease (ZFN) gene editing techniques. Still another embodiment of this aspect that can be combined with any of the preceding embodiments that has a gene editing technique includes the one or more genetic alterations that increase the activity of the endogenous PsbS protein being selected from the group of inactivating a repressor element that represses expression of the endogenous PsbS protein, removing a repressor element that represses expression of the endogenous PsbS protein, modulating the methylation state of a repressor element that represses expression of the endogenous PsbS protein, activating an enhancer element that increases expression of the endogenous PsbS protein, adding an enhancer element that increases expression of the endogenous PsbS protein, modulating the methylation state of an enhancer element that increases expression of the endogenous PsbS protein, adding a transcriptional activator recruiting or binding element that activates expression of the endogenous PsbS protein, replacing the endogenous promoter with an overexpression promoter that directs expression of the endogenous PsbS protein, modulating the methylation state of the endogenous promoter; modulating the methylation state of the endogenous PsbS coding sequence; adding elements that stabilize an endogenous PsbS mRNA, removing elements that destabilize the endogenous PsbS mRNA, modifying a PsbS coding sequence to increase stability of the PsbS protein, or modifying a PsbS coding sequence to increase activity of the PsbS protein.

In still another embodiment of this aspect that can be combined with any of the preceding embodiments, the PsbS protein is localized to a thylakoid membrane of at least one chloroplast within a cell of the genetically altered plant. An additional embodiment of this aspect includes the cell being a chloroplast containing leaf cell. A further embodiment of this aspect includes the cell being a mesophyll cell or a guard cell. In yet another embodiment of this aspect that can be combined with any of the preceding embodiments that has localization of the PsbS protein, the PsbS protein is expressed in at least 70% of the cells.

Yet another embodiment of this aspect that can be combined with any of the relevant preceding embodiments with respect to a genetically altered plant includes a genetically altered plant produced from the method of any one of the preceding embodiments. An additional embodiment of this aspect includes a genetically altered plant part produced from the genetically altered plant, wherein the plant part is a leaf, a stem, a root, a flower, a seed, a kernel, a grain, a pod, a bean, a pea, a fruit, a chloroplast, a cell, or a portion thereof and the genetically altered plant part includes the one or more genetic alterations. A further embodiment of this aspect includes the plant part being a fruit, a kernel, a grain, a pod, a bean, or a pea. Still another embodiment of this aspect includes a genetically altered pollen grain or a genetically altered ovule of the genetically altered plant, wherein the genetically altered pollen grain or the genetically altered ovule includes the one or more genetic alterations. Yet another embodiment of this aspect includes a genetically altered protoplast produced from the genetically altered plant, wherein the genetically altered protoplast includes the one or more genetic alterations. A further embodiment of this aspect includes a genetically altered tissue culture produced from protoplasts or cells from the genetically altered plant wherein the cells or protoplasts are produced from a plant part selected from the group of leaf, leaf mesophyll cell, anther, pistil, stem, petiole, root, root tip, fruit, seed, kernel, grain, pod, bean, pea, flower, cotyledon, hypocotyl, embryo, or meristematic cell, wherein genetically altered tissue culture includes the one or more genetic alterations. An additional embodiment of this aspect includes a genetically altered plant regenerated from the genetically altered tissue culture that includes the one or more genetic alterations. In still another embodiment of this aspect, the genetically altered plant regenerated from the genetically altered tissue culture has all the physiological and morphological characteristics of the genetically altered plant produced from the method of any one of the preceding embodiments.

An additional aspect of the disclosure includes a genetically altered plant or part thereof including one or more genetic alterations that increase activity of a PsbS protein as compared to a WT plant without the one or more genetic alterations, wherein the genetically altered plant shows increased water use efficiency as compared to the WT plant grown under the same conditions. An additional embodiment of this aspect includes the conditions being selected from the group of reduced irrigation conditions, rain fed conditions, high density growth conditions, mild salinity, fertilized or providing additional nutrients, humid conditions, or conditions resulting in wet leaf surfaces. In yet another embodiment of this aspect, which may be combined with any of the preceding embodiments, the increased activity of the PsbS protein provides the genetically altered plant with a higher yield, an increased biomass, an increased growth rate, an increased tolerance of salinity, an increased ability to withstand salinity, an increased flow of nutrients to the roots, an increased availability of nutrients over time, an increased utilization of fertilizer, an increased utilization of nutrients, a decreased susceptibility to a plant disease requiring humid conditions and/or wet leaf surfaces for infection, a decreased susceptibility to infection by the plant disease, a reduced incidence of the plant disease, or a reduced incidence of infection by the plant disease as compared to the WT plant without the increased activity grown under the same conditions where the activity of the PsbS protein is increased. In still another embodiment of this aspect that can be combined with any of the preceding embodiments, the genetically altered plant does not include increased activity of proteins ZEP, VDE, or both, as compared to a WT plant. In an additional embodiment of this aspect that can be combined with any of the preceding embodiments, the genetically altered plant does not include reduced activity of KEA3 as compared to a WT plant.

In still another embodiment of this aspect that can be combined with any of the relevant preceding embodiments, the genetically altered plant further includes increased activity of a ZEP protein and increased activity of a VDE protein, wherein the increased activity of the PsbS protein is greater than the increased activity of the PsbS protein required for increased photosynthetic efficiency when each of the ZEP protein, the PsbS protein, and the VDE protein are overexpressed together. An additional embodiment of this aspect includes increased activity of the PsbS protein being due to an increased amount of the PsbS protein. In yet another embodiment of this aspect, the increased amount of PsbS protein is at least 10% greater, at least 20% greater, at least 30% greater, at least 40% greater, at least 50% greater, at least 75% greater, at least 100% greater, at least 150% greater, or at least 200% greater than the increased activity of the PsbS protein required for increased photosynthetic efficiency when each of the ZEP protein, the PsbS protein, and the VDE protein are overexpressed together. Still another embodiment of this aspect includes the increased amount of PsbS protein being no greater than 500%, no greater than 400%, no greater than 300%, no greater than 200%, no greater than 150%, no greater than 100%, no greater than 75%, or no greater than 50%, of the increased activity of the PsbS protein required for increased photosynthetic efficiency when each of the ZEP protein, the PsbS protein, and the VDE protein are overexpressed together. Another embodiment of this aspect includes the increased amount of PsbS, the increased amount of ZEP, and the increased amount of VDE being increased relative to an amount of endogenous PsbS protein, an amount of endogenous ZEP protein, and an amount of endogenous VDE protein, and wherein the increased amount of PsbS is 10% or more, 12% or more, 14% or more, 16% or more, 18% or more, 20% or more, 22% or more, 24% or more, 30% or more, 40% or more, 50% or more, 100% or more, or 200% or more increased relative to the amount of endogenous PsbS protein, the increased amount of ZEP is 90% increased relative to the amount of endogenous ZEP protein, and the increased amount of VDE is 80% increased relative to the amount of endogenous VDE protein. In a further embodiment of this aspect, the increased amount of PsbS, the increased amount of ZEP, and the increased amount of VDE is increased relative to an amount of endogenous PsbS protein, an amount of endogenous ZEP protein, and an amount of endogenous VDE protein, and wherein the increased amount of PsbS is 12% increased relative to the amount of endogenous PsbS protein, the increased amount of ZEP is 90% increased relative to the amount of endogenous ZEP protein, and the increased amount of VDE is 80% increased relative to the amount of endogenous VDE protein. In yet another embodiment of this aspect, which may be combined with any of the preceding embodiments that has increased activity of a ZEP protein and increased activity of a VDE protein, the increased activity of the PsbS, VDE, and ZEP proteins provides the genetically altered plant with increased photosynthetic efficiency under non-steady state conditions as compared to the WT plant without the increased activity grown under the same conditions where the activity of the PsbS, VDE, and ZEP proteins is increased.

Still another embodiment of this present aspect, which may be combined with any of the preceding embodiments, includes the genetically altered plant being selected from the group of cowpea, soybean, cassava, wheat, barley, corn, sorghum, rice, cotton, sugarcane, eucalyptus, poplar, willow, orange, grapefruit, lemon, lime, avocado, cherry, peach, plum, apricot, nectarine, fig, olive, almond, pistachio, walnut, chestnut, hazelnut, pecan, tomato, eggplant, potato, or alfalfa. An additional embodiment of this aspect includes the genetically altered plant not being rice or Arabidopsis.

Still another embodiment of this aspect that can be combined with any of the preceding embodiments includes the increased activity of the PsbS protein providing the genetically altered plant with decreased stomatal conductance as compared to the WT plant without the increased activity grown under the same conditions where the activity of the PsbS protein is increased. In yet another embodiment of this aspect, which may be combined with any of the preceding embodiments that does not have increased activity of a ZEP protein and increased activity of a VDE protein, the increased activity of the PsbS protein provides the genetically altered plant with substantially similar photosynthetic efficiency as compared to the WT plant grown under the same conditions where the activity of the PsbS protein is increased.

In a further embodiment of this aspect that can be combined with any of the preceding embodiments, increased activity is increased expression. An additional embodiment of this aspect includes the increased expression being due to expression of a heterologous PsbS protein. A further embodiment of this aspect includes the heterologous PsbS protein including an amino acid sequence with at least 70% sequence identity to, at least 75% sequence identity to, at least 80% sequence identity to, at least 85% sequence identity to, at least 90% sequence identity to, at least 95% sequence identity to, or at least 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184. Yet another embodiment of this aspect includes the heterologous PsbS protein being selected from the group of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184. A further embodiment of this aspect that can be combined with any of the preceding embodiments that has a heterologous PsbS protein includes the heterologous PsbS protein containing a glutamate at a position corresponding to amino acid 149 of reference sequence SEQ ID NO: 21 and a glutamate at a position corresponding to amino acid 255 of reference sequence SEQ ID NO: 21. Still another embodiment of this aspect that can be combined with any of the preceding embodiments that has a heterologous PsbS protein includes the heterologous PsbS protein being encoded by a first nucleic acid and the first nucleic acid being operably linked to a second nucleic acid including a promoter. An additional embodiment of this aspect includes the promoter being selected from the group of a CaMV35S promoter, a ubiquitin promoter, a Rbcs1A promoter, a GAPA-1 promoter, a FBA2 promoter, or any combination thereof. Still another embodiment of this aspect that can be combined with any of the preceding embodiments that has a nucleic acid sequence includes the first nucleic acid sequence and the second nucleic acid sequence being stably integrated into a nuclear genome of the plant or into a chloroplast genome of the plant. A further embodiment of this aspect includes the increased expression being due to overexpression of an endogenous PsbS protein. An additional embodiment of this aspect includes overexpression of the endogenous PsbS protein being achieved using a gene editing technique to introduce the one or more genetic alterations that increase the activity of the endogenous PsbS protein. Still another embodiment of this aspect includes the gene editing technique being selected from the group of transcription activator-like effector nuclease (TALEN) gene editing techniques, clustered Regularly Interspaced Short Palindromic Repeat (CRISPR/Cas) gene editing techniques, or zinc-finger nuclease (ZFN) gene editing techniques. Still another embodiment of this aspect that can be combined with any of the preceding embodiments that has a gene editing technique includes the one or more genetic alterations that increase the activity of the endogenous PsbS protein are selected from the group of inactivating a repressor element that represses expression of the endogenous PsbS protein, removing a repressor element that represses expression of the endogenous PsbS protein, modulating the methylation state of a repressor element that represses expression of the endogenous PsbS protein, activating an enhancer element that increases expression of the endogenous PsbS protein, adding an enhancer element that increases expression of the endogenous PsbS protein, modulating the methylation state of an enhancer element that increases expression of the endogenous PsbS protein, adding a transcriptional activator recruiting or binding element that activates expression of the endogenous PsbS protein, replacing the endogenous promoter with an overexpression promoter that directs expression of the endogenous PsbS protein, modulating the methylation state of the endogenous promoter; modulating the methylation state of the endogenous PsbS coding sequence; adding elements that stabilize an endogenous PsbS mRNA, removing elements that destabilize the endogenous PsbS mRNA, modifying a PsbS coding sequence to increase stability of the PsbS protein, or modifying a PsbS coding sequence to increase activity of the PsbS protein.

In still another embodiment of this aspect that can be combined with any of the preceding embodiments, the PsbS protein is localized to a thylakoid membrane of at least one chloroplast within a cell of the genetically altered plant. An additional embodiment of this aspect includes the cell being a chloroplast containing leaf cell. A further embodiment of this aspect includes the cell being a mesophyll cell or a guard cell. In yet another embodiment of this aspect that can be combined with any of the preceding embodiments that has localization of the PsbS protein, the PsbS protein is expressed in at least 70% of the cells.

Yet another embodiment of this aspect that can be combined with any of the preceding embodiments with respect to seed includes a genetically altered seed produced from the genetically altered plant of any one of the preceding embodiments. Still another embodiment of this aspect that can be combined with any of the preceding embodiments with respect to plant part includes the plant part being a leaf, a stem, a root, a flower, a seed, a kernel, a grain, a pod, a bean, a pea, a fruit, a chloroplast, a cell, or a portion thereof and the genetically altered plant part including the one or more genetic alterations. A further embodiment of this aspect includes the plant part being a fruit, a kernel, a grain, a pod, a bean, or a pea. Still another embodiment of this aspect that can be combined with any of the preceding embodiments with respect to pollen grain or ovules includes a genetically altered pollen grain or a genetically altered ovule of the plant of any one of the preceding embodiments, wherein the genetically altered pollen grain or the genetically altered ovule includes the one or more genetic alterations. A further embodiment of this aspect that can be combined with any of the preceding embodiments includes a genetically altered protoplast produced from the genetically altered plant of any of the preceding embodiments, wherein the genetically altered protoplast includes the one or more genetic alterations. An additional embodiment of this aspect that can be combined with any of the preceding embodiments includes a genetically altered tissue culture produced from protoplasts or cells from the genetically altered plant of any one of the preceding embodiments, wherein the cells or protoplasts are produced from a plant part selected from the group of leaf, leaf mesophyll cell, anther, pistil, stem, petiole, root, root tip, tuber, fruit, seed, kernel, grain, flower, cotyledon, hypocotyl, embryo, or meristematic cell, wherein the genetically altered tissue culture includes the one or more genetic alterations. An additional embodiment of this aspect includes a genetically altered plant regenerated from the genetically altered tissue culture that includes the one or more genetic alterations. In still another embodiment of this aspect, the genetically altered plant regenerated from the genetically altered tissue culture has all the physiological and morphological characteristics of the genetically altered plant produced from the method of any one of the preceding embodiments.

An additional aspect of the disclosure includes methods of cultivating the genetically altered plant of any of the preceding embodiments including the steps of (a) planting a genetically altered seedling, a genetically altered plantlet, a genetically altered cutting, a genetically altered tuber, a genetically altered root, or a genetically altered seed to produce the genetically altered plant, or by grafting the genetically altered seedling, the genetically altered plantlet, or the genetically altered cutting to a root stock or a second plant to produce the genetically altered plant; (b) cultivating the genetically altered plant to produce harvestable seed and fruits or vegetatively produced harvested items, wherein the harvestable seed and fruits is selected from seed, fruit, pods, grain, kernels, beans, and peas, and wherein the vegetatively produced harvested items are selected from tubers, rhizomes, buds, roots, cuttings, and leaves; and (c) harvesting the harvestable seed, harvestable leaves, harvestable roots, harvestable cuttings, harvestable fruit, harvestable kernels, harvestable tubers, harvestable pods, harvestable peas, harvestable beans, and/or harvestable grain.

An additional aspect of the disclosure includes methods of producing the genetically altered plant of any of the preceding embodiments including the steps of (a) transforming a plant cell, tissue, or other explant with a vector including a first nucleic acid sequence encoding a PsbS protein operably linked to a second nucleic acid sequence encoding a promoter; (b) selecting successful transformation events by means of a selection agent, marker-assisted selection, or selective media; (c) regenerating the transformed cell, tissue, or other explant into a genetically altered plantlet; and (d) growing the genetically altered plantlet into a genetically altered plant with increased activity of a PsbS protein as compared to an untransformed WT plant. An additional embodiment of this aspect further includes identifying successful introduction of the one or more genetic alterations by screening or selecting the plant cell, tissue, or other explant prior to step (b); screening or selecting plantlets between step (b) and (c); or screening or selecting plants after step (c). In yet another embodiment of this aspect, which may be combined with any of the preceding embodiments, transformation is done using a transformation method selected from the group of particle bombardment (i.e., biolistics, gene gun), Agrobacterium-mediated transformation, Rhizobium-mediated transformation, or protoplast transfection or transformation. In still another embodiment of this aspect, which may be combined with any of the preceding embodiments, the vector is pEG100. A further embodiment of this aspect includes the PsbS protein including an amino acid sequence with at least 70% sequence identity to, at least 75% sequence identity to, at least 80% sequence identity to, at least 85% sequence identity to, at least 90% sequence identity to, at least 95% sequence identity to, or at least 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184. Yet another embodiment of this aspect includes the PsbS protein being selected from the group of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184. A further embodiment of this aspect that can be combined with any of the preceding embodiments includes the PsbS protein containing a glutamate at a position corresponding to amino acid 149 of reference sequence SEQ ID NO: 21 and a glutamate at a position corresponding to amino acid 255 of reference sequence SEQ ID NO: 21. Still another embodiment of this aspect that can be combined with any of the preceding embodiments includes the heterologous PsbS protein being encoded by a first nucleic acid and the first nucleic acid being operably linked to a second nucleic acid including a promoter. An additional embodiment of this aspect includes the promoter being selected from the group of a CaMV35S promoter, a ubiquitin promoter, a Rbcs1A promoter, a GAPA-1 promoter, a FBA2 promoter, or any combination thereof. Still another embodiment of this aspect that can be combined with any of the preceding embodiments that has a nucleic acid sequence includes the first nucleic acid sequence and the second nucleic acid sequence being stably integrated into a nuclear genome of the plant or into a chloroplast genome of the plant.

A further aspect of the disclosure includes methods of producing the genetically altered plant of any of the preceding embodiments, including the steps of (a) transforming a plant cell, tissue, or other explant with one or more gene editing components that target a nuclear genome sequence operably linked to an endogenous PsbS protein; (b) selecting successful transformation events by means of a screening technology, an enriching technology, a selection agent, marker-assisted selection, or selective media; (c) regenerating the transformed cell, tissue, or other explant into a genetically altered plantlet; and (d) growing the genetically altered plantlet into a genetically altered plant with increased activity of a PsbS protein as compared to an untransformed WT plant. An additional embodiment of this aspect includes the one or more gene editing components including a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

Still another aspect of the disclosure includes methods of identifying genetic markers associated with increased PsbS protein activity in a plant, including the steps of (a) screening a population of plants from the same species or closely related species for PsbS protein activity; (b) identifying a subset of plants from the population with higher levels of PsbS activity as compared to the other plants in the population; and (c) identifying genetic markers associated with increased PsbS activity in the subset of plants. An additional embodiment of this aspect includes the increased PsbS protein activity being due to increased expression of a PsbS mRNA and the screening in step (a) being assaying levels of the PsbS mRNA, optionally using a method selected from the group of RNA-Seq, microarray, Northern blot, or qRT-PCR. A further embodiment of this aspect includes the increased PsbS protein activity being due to increased amount of the PsbS protein and the screening in step (a) being assaying levels of the PsbS protein, optionally using a method selected from the group of Western blot, ELISA, immunoprecipitation, HPLC, or LC/MS.

Yet another aspect of the disclosure includes methods of producing a plant with increased WUE activity and a second desired trait, including the steps of (a) providing a first plant including a genetic marker associated with increased PsbS activity and a second plant including the second desired trait; (b) crossing the first plant with the second plant to create a population of progeny plants; and (c) selecting the plant with increased WUE activity and the second desired trait using the genetic marker associated with increased PsbS activity. An additional embodiment of this aspect includes the second desired trait being increased PsbS activity unlinked to the genetic marker associated with increased PsbS activity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B show NPQ levels under different conditions. FIG. 1A shows NPQ as a function of incident light intensity in WT and mutant N. tabacum lines grown under controlled conditions. Open circles represent partially silenced PsbS mutant line psbs-4, gray circles represent WT, red triangles represent PsbS overexpression line PSBS-28, and red circles represent PsbS overexpression line PSBS-43. (Asterisks/lines show significant differences from WT (P<0.0001), black for silencing, red for overexpressing lines; Dunnett's two-way test; α=0.05; error bars indicate standard error of the mean (s.e.m.); n=6 to 10 biological replicates). FIG. 1B shows NPQ levels in leaf discs of WT and mutant N. tabacum lines grown under field-test conditions.

FIGS. 2A-2B show PsbS mRNA expression. FIG. 2A shows relative PsbS mRNA expression normalized to actin and tubulin for WT and mutant N. tabacum lines grown under controlled conditions. The white column represents partially silenced PsbS mutant line psbs-4, the gray column represents WT, and the red columns represent PsbS overexpression lines PSBS-28 and PSBS-43. (Asterisks show significant differences from WT (P<0.0001); Dunnett's two-way test; α=0.05; error bars indicate s.e.m.; n=6 to 10 biological replicates). FIG. 2B shows a developmental time-course of relative PsbS mRNA expression normalized to actin and tubulin, which were used as internal standards, in WT and mutant N. tabacum lines grown under field-test conditions. The white column represents partially silenced PsbS mutant line psbs-4, the gray column represents WT, and the red columns represent PsbS overexpression lines PSBS-28, PSBS-34, PSBS-43, and PSBS-46. (All genotypes significantly different from WT; Dunnett's two-way test; p≤0.008; error bars indicate s.e.m; n=4 biological replicates).

FIGS. 3A-3C show PsbS protein expression. FIG. 3A shows PsbS protein expression normalized to PsbO expression, which was used as an internal control, as determined from immunoblot densitometry readings for WT and mutant N. tabacum lines grown under controlled conditions. The white column represents partially silenced PsbS mutant line psbs-4, the gray column represents WT, and the red columns represent PsbS overexpression lines PSBS-28 and PSBS-43. (Asterisks show significant differences from WT (P<0.0001); Dunnett's two-way test; α=0.05; error bars indicate s.e.m.; n=6 to 10 biological replicates). FIG. 3B shows a representative immunoblot for PsbS and PsbO protein expression in WT and mutant N. tabacum plant lines grown under control conditions. FIG. 3C shows a developmental time-course of PsbS protein expression normalized to PsbO expression, which was used as an internal control, in WT and mutant N. tabacum lines grown under field-test conditions. The white column represents partially silenced PsbS mutant line psbs-4, the gray column represents WT, and the red columns represent PsbS overexpression lines PSBS-28, PSBS-34, PSBS-43, and PSBS-46. (All genotypes significantly different from WT; Dunnett's two-way test; p≤0.001; error bars indicate s.e.m; n=4 biological replicates).

FIGS. 4A-4E show measurements of photosynthesis and stomatal conductance in PsbS suppressed lines, PsbS overexpression lines, and WT under controlled conditions. FIG. 4A shows net CO₂ assimilation (Aₙ) as a function of incident light intensity in WT and mutant N. tabacum lines grown under controlled conditions (P=0.60). FIG. 4B shows stomatal conductance as a function of incident light intensity in WT and mutant N. tabacum lines grown under controlled conditions (P=0.73). FIG. 4C shows a strong positive correlation between water use efficiency (net CO₂ assimilation (Aₙ)/stomatal conductance (gₛ)) and PsbS expression (R²=0.92; P=0.03). Thus, plants with increased PsbS expression have improved WUE. FIG. 4D shows redox state of plastoquinone (Q_{A}) as a function of incident light intensity in WT and mutant N. tabacum lines grown under controlled conditions (P=0.0001). FIG. 4E shows a highly significant positive correlation and linear relationship between Q_{A} redox state and stomatal conductance (gₛ) in WT and mutant N. tabacum lines grown under controlled conditions (R²=0.98; P<0.0001). (Broken lines indicate measurements at highest light intensity). For all figures, open circles represent partially silenced PsbS mutant line psbs-4, gray circles represent WT, red triangles represent PsbS overexpression line PSBS-28, and red circles represent PsbS overexpression line PSBS-43. (Asterisks/lines show significant differences from WT, black for silencing, red for overexpressing lines; Dunnett's two-way test; α=0.05; error bars indicate s.e.m.; n=6 to 10 biological replicates).

FIGS. 5A-5D show measurements of stomatal characteristics in PsbS mutant lines, PsbS overexpressing lines, and WT. FIG. 5A shows stomatal density (abaxial leaf side P=0.006; adaxial leaf side P=0.02), FIG. 5B shows stomatal complex width (abaxial leaf side P=0.62; adaxial leaf side P=0.41), FIG. 5C shows stomatal complex length (abaxial leaf side P=0.96; adaxial leaf side P=0.69), and FIG. 5D shows stomatal complex width×length (abaxial leaf side P=0.92; adaxial leaf side P=0.53) in WT and mutant N. tabacum lines grown under controlled conditions. The white column represents partially silenced PsbS mutant line psbs-4, the gray column represents WT, the red column represents PsbS overexpression line PSBS-28, and the red lined column represent PsbS overexpression line PSBS-43. (Asterisks show significant differences from WT; Dunnett's two-way test; α=0.05; error bars indicate s.e.m.; n=4 biological replicates).

FIGS. 6A-6F show measurements of photosynthesis and stomatal conductance in PsbS suppressed lines, PsbS overexpression lines, and WT under field-test conditions. FIG. 6A shows net CO₂ assimilation (Aₙ) as a function of incident light intensity in WT and mutant N. tabacum lines grown under field-test conditions. FIG. 6B shows net CO₂ assimilation (Aₙ) in mutant N. tabacum lines relative to WT grown under field-test conditions (P=0.96). FIG. 6C shows stomatal conductance (gₛ) as a function of incident light intensity in WT and mutant N. tabacum lines grown under field-test conditions. The effect on gₛ is much larger than the effect on CO₂ assimilation (Aₙ) shown in FIG. 6A. FIG. 6D shows stomatal conductance (gₛ) in mutant N. tabacum lines relative to WT grown under field-test conditions (P=0.0001). FIG. 6E shows water use efficiency (Aₙ/gₛ) in mutant N. tabacum lines relative to WT grown under field-test conditions (P=0.007). FIG. 6F shows a strong positive correlation between water use efficiency (Aₙ/gₛ) and relative PsbS expression (R²=0.94; P=0.004). For FIGS. 6A, 6C, and 6F, open circles represent partially silenced PsbS mutant line psbs-4, gray circles represent WT, red triangles represent PsbS overexpression line PSBS-28, red diamonds represent PsbS overexpression line PSBS-34, and red circles represent PsbS overexpression line PSBS-43. For FIGS. 6B, 6D, and 6E, the white column represents partially silenced PsbS mutant line psbs-4 and the red columns represent PsbS overexpression lines PSBS-28, PSBS-34, and PSBS-43. (Error bars indicate s.e.m.; n=4 biological replicates).

FIGS. 7A-7G show comparative measurements of a PsbS mutant line, two PsbS overexpression lines, and the WT plant for various photosynthetic components and their biochemical characteristics. FIG. 7A shows CO₂ fixation rate as a function of chloroplastic CO₂ concentration in WT and mutant N. tabacum lines grown under controlled conditions. FIG. 7B shows electron transport rate as a function of incident light intensity in WT and mutant N. tabacum lines grown under controlled conditions. FIG. 7C shows maximum ribulose bisphosphate carboxylation capacity (P=0.29), FIG. 7D shows maximum rate linear electron transport (P=0.07), FIG. 7E shows Rubisco content (P=0.90), FIG. 7F Rubisco activation state (P=0.06), and FIG. 7G shows stomatal limitation in the youngest fully expanded leaves of WT and mutant N. tabacum lines grown under controlled conditions (P=0.006). For FIGS. 7A and 7B, open circles represent partially silenced PsbS mutant line psbs-4, gray circles represent WT, red triangles represent PsbS overexpression line PSBS-28, and red circles represent PsbS overexpression line PSBS-43. For FIGS. 7C-7G, the white column represents partially silenced PsbS mutant line psbs-4, the gray column represents WT, and the red columns represent PsbS overexpression lines PSBS-28 and PSBS-43. (Error bars indicate s.e.m.; n=6-10 biological replicates for FIGS. 7A-7D and 7G; n=3 biological replicates for FIG. 7E; n=4 biological replicates for FIG. 7F).

FIGS. 8A-8F show plant biomass measurements in PsbS suppressed lines, PsbS overexpression lines, and WT grown under fully irrigated conditions. FIG. 8A shows total dry weight (P=0.0001), FIG. 8B shows root dry weight (P=0.001), FIG. 8C shows stem dry weight (P=0.0006), FIG. 8D shows leaf dry weight (P=0.0005), FIG. 8E shows leaf area (P=0.008), and FIG. 8F shows plant height in mutant N. tabacum lines relative to WT grown under field-test conditions (P<0.0001). The white columns represent partially silenced PsbS mutant lines psbs-4 and psbs-50 and the red columns represent PsbS overexpression lines PSBS-28, PSBS-34, PSBS-43, and PSBS-46. (Asterisks indicate significant differences between transgenic and WT; Dunnett's two-way test; α=0.05; error bars indicate s.e.m.; n=6 blocks for transgenics and n=12 blocks for WT).

FIG. 9 shows the plasmid map for pEG100-NbPsbS used to transform N. tabacum.

FIGS. 10A-10E show the alignment of PsbS polypeptide sequences from Arabidopsis thaliana (At; SEQ ID NO: 1), Oryza sativa (Os; SEQ ID NO: 2, SEQ ID NO: 3, SEQ ID NO: 4, SEQ ID NO: 5, SEQ ID NO: 6), Vigna unguiculata (Vg; SEQ ID NO: 7), Glycine max (Gm; SEQ ID NO: 8, SEQ ID NO: 9, SEQ ID NO: 10, SEQ ID NO: 11), Zea mays (Zm; SEQ ID NO: 12, SEQ ID NO: 13, SEQ ID NO: 14), Nicotiana tabacum (Nt; SEQ ID NO: 15, SEQ ID NO: 16), Nicotiana benthamiana (Nb; SEQ ID NO: 17, SEQ ID NO: 18), Hevea brasiliensis (Hb; SEQ ID NO: 19), and Manihot esculenta (Me; SEQ ID NO: 20). FIG. 10A shows the alignment of the N terminal portion of the PsbS protein (Os_BAA12337.1, Os_CAH68096.1, Os_CAE01809.2, Os_XP_015633953.1, Os_XP_015621169.1, Zm_ACG37564.1, Zm_NP_001105228.2, Zm_AAQ55066.1, Nb_ABC59516.1, Nt_NP_001312190.1, Nt_XP_016484565.1, Nb_ABC59515.1, At_spIQ9XF91|PSBS_ARATH, Hb_XP_021647432.1, Me_Cassava_V7_genomic_Chrl8_8840000to8855000_rev, Vg_VigunO9g165900.1, Gm_XP_003523444.1, Gm_ACU23291.1, Gm_KRH53237.1, and Gm_NP_001276237.1, SEQ ID NOs:185-204, respectively). FIG. 10B shows the alignment of the central portion of the PsbS protein (Os_BAA12337.1, Os_CAH68096.1, Os_CAE01809.2, Os_XP_015633953.1, Os_XP_015621169.1, Zm_ACG37564.1, Zm_NP_001105228.2, Zm_AAQ55066.1, Nb_ABC59516.1, Nt_NP_001312190.1, Nt_XP_016484565.1, Nb_ABC59515.1, At_spIQ9XF91|PSBS_ARATH, Hb_XP_021647432.1, Me_Cassava_V7_genomic_Chr18_8840000to8855000_rev, Vg_Vigun09g165900.1, Gm_XP_003523444.1, Gm_ACU23291.1, Gm_KRH53237.1, and Gm_NP_001276237.1,SEQ ID NOs:205-224, respectively). FIG. 10C continues the alignment of the central portion of the PsbS protein (Os_BAA12337.1, Os_CAH68096.1, Os_CAE01809.2, Os_XP_015633953.1, Os_XP_015621169.1, Zm_ACG37564.1, Zm_NP_001105228.2, Zm_AAQ55066.1, Nb_ABC59516.1, Nt_NP_001312190.1, Nt_XP_016484565.1, Nb_ABC59515.1, At_spIQ9XF91|PSBS_ARATH, Hb_XP_021647432.1, Me_Cassava_V7_genomic_Chr18_8840000to8855000_rev, Vg_VigunO9g165900.1, Gm_XP_003523444.1, Gm_ACU23291.1, Gm_KRH53237.1, and Gm_NP_001276237.1, SEQ ID NOs:225-244, respectively). FIG. 10D continues the alignment of the central portion of the PsbS protein (Os_BAA12337.1, Os_CAH68096.1, Os_CAE01809.2, Os_XP_015633953.1, Os_XP_015621169.1, Zm_ACG37564.1, Zm_NP_001105228.2, Zm_AAQ55066.1, Nb_ABC59516.1, Nt_NP_001312190.1, Nt_XP_016484565.1, Nb_ABC59515.1, At_spIQ9XF91|PSBS_ARATH, Hb_XP_021647432.1, Me_Cassava_V7_genomic_Chr18_8840000to8855000_rev, Vg_Vigun09g165900.1, Gm_XP_003523444.1, Gm_ACU23291.1, Gm_KRH53237.1, Gm_NP_001276237.1 (SEQ ID NOs:245-264, respectively). FIG. 10E shows the alignment of the C terminal portion of the PsbS protein (Os_BAA12337.1, Os_CAH68096.1, Os_CAE01809.2, Os_XP_015633953.1, Os_XP_015621169.1, Zm_ACG37564.1, Zm_NP_001105228.2, Zm_AAQ55066.1, Nb_ABC59516.1, Nt_NP_001312190.1, Nt_XP_016484565.1, Nb_ABC59515.1, At_spIQ9XF91PSBS_ARATH, Hb_XP_021647432.1, Me_Cassava_V7_genomic_Chr18_8840000to8855000_rev, Vg_Vigun09g165900.1, Gm_XP_003523444.1, Gm_ACU23291.1, Gm_KRH53237.1, and Gm_NP_001276237.1, SEQ ID NOs:265-284, respectively). In FIGS. 10A-10E, the amino acids are color coded by identity. In FIG. 10C and FIG. 10E, a green box indicates the location of a glutamate (E) within a short loop sequence that is located in the chloroplast lumen when the PsbS protein is expressed.

### DETAILED DESCRIPTION

This disclosure is based, in part, upon the inventors' identification of a surprising new utility for increased PsbS activity that results in plants with reduced stomatal conductance and increased water use efficiency. This surprising new utility for PsbS identified by the inventors serves as the basis for many of the aspects and their various embodiments of the present disclosure. Stomatal opening and closing is regulated by a variety of environmental cues in order to balance water vapor efflux and CO₂ influx. Some of these environmental cues include CO₂ concentration and light intensity, particularly of blue and red light. Although progress has been made in unraveling the molecular mechanisms underlying stomatal response to CO₂ concentration and blue light, the molecular mechanisms underlying stomatal response to red light remain elusive.

One possible molecule involved in the stomatal response to red light is chloroplastic plastoquinone (Q_{A}). A correlation between the redox state of Q_{A} and stomatal conductance has been shown, suggesting that the Q_{A} redox state may serve as a photosynthetic signal that controls stomatal opening in response to red light. However, no causative link between Q_{A} redox state and stomatal conductance had been established.

Without wanting to be limited by theory, increasing PsbS activity may exert its effect by regulating Q_{A} redox state. Moreover, PsbS acts on light harvesting and energy dissipation, which impacts Q_{A} redox state, and thereby may indirectly link regulation of photosynthetic light harvesting to stomatal regulation.

### Growth Conditions

Reductions in the inherent water use (i.e., increased WUE) per unit of growth and/or harvest of any crop can increase yield, and reduce cost and risk, especially under conditions without other rate-limiting factors. Under irrigation, crops that require less water per unit of yield will require less irrigation, saving water cost, and reducing the risk of loss from short, local water stress conditions between rain and irrigation cycles. Under rain-fed conditions where choice of planting density, variety maturity group and other management choices are optimized to the expected total seasonal water availability, crops that require less water can be managed more intensively for greater yield per acre—primarily by increasing plant density, but under certain conditions also choosing longer season varieties or more cost-effective fertilizer regimes, both of which can be influenced by seasonal water availability parameters. Even the effective growing range of a valuable crop can be extended into more marginal areas by reducing the seasonal water needs of the crop.

A particular feature of many crops such as corn, soybean and others is a heightened sensitivity to water stress at particular times during their growth cycle, where a short period of water stress can disproportionately reduce yield compared to a similar short period of water stress during other parts of the growing season. Conserving soil water, which can be achieved by growing plants of the current invention, would help to bridge these most damaging short periods of water stress between rainfall variations. Further, a study done on corn yields showed that increased yields were associated with increased sensitivity to drought stress (Lobell et al. (2014) Greater Sensitivity to Drought Accompanies Maize Yield Increase in the US Midwest. Science, 344, 516-519). Plants of the current invention, which have increased WUE, could reduce drought sensitivity of yields at the field scale.

One of skill in the art would know that the choice of planting density and fertilizer rate are affected by the chosen water regime. A study done in cowpea (Vigna unguiculata L. Walp.) showed that water limitation decreased yield, and further showed that the interaction between moisture and planting density was significant for measures such as total biomass (Lemma et al., Journal of Agronomy, 2008). This study indicates the importance of employing agricultural practices, such as planting density, to optimally utilize available moisture. This is particularly true under rain fed conditions, which require knowledge of the expected seasonal rainfall and the water available in the soil at the beginning of the planting season (i.e., the soil water bank). One of skill in the art would utilize available tools to quantify when water availability is limiting plant yield or growth (e.g., Friedman, Soil Science of America, 2016). One of skill in the art would further understand that the plants of the present invention keep more water in the soil water bank. Therefore, the plants of the current invention allow planting at a higher density with more fertilizer. In addition, this means that there are less local stress conditions within the field. The reduction of local stress conditions results in a higher yield at the end of the season.

Further, one of skill in the art would know that decisions regarding water regime application are dependent on the crop species. For example, a crop producing smaller plants will require less water than a crop producing larger plants, as leaf area, and therefore transpiration, will also be smaller. Similarly, a crop growing for a full season will require more water than a crop growing for part of a season. One of skill in the art would also know that water requirements are also dependent on the developmental stage of a crop. Early growth stages often do not require irrigation, but can be rain fed and use the soil water bank. Vegetative growth stages may need to be irrigated, but are able to survive if water stress should occur. Generally, reproductive growth stages require the most water, and the water available during this stage directly affects yield. One of skill in the art would know that reproductive growth stages are sensitive to water availability, and that in particular the early reproductive growth stages are the critical time when water is required.

The environment during the growing season will also affect water requirements. Factors such as heat, sun, humidity, and wind can all affect availability of water. One of skill in the art would be aware that a hot, sunny day with high winds and low humidity will result in more soil water evaporation than a cool, overcast day with high humidity and no wind.

Finally, the characteristics of the soil also contribute to crop water requirements. One of skill in the art would know that soils with a fine texture are able to hold more water than soils with a coarse texture. As described above, one of skill in the art would also be aware of the importance of the soil water bank for plant water availability. In addition, one of skill in the art would know that soil water evaporation can be reduced by agricultural practices such as conservation tillage and increased surface crop residue. Tillage is known to expose soil surface area, increase runoff, increase evaporation, remove remnants of previous crops that could have caught precipitation, and to compact some areas of soil, thereby potentially reducing the amount of water able to enter the soil. Therefore, conservation tillage can reduce these effects. Similarly, residue on the soil surface can increase water infiltration by preventing surface sealing due to water droplets. Residue on the soil surface also catches water and prevents it from running off, giving the water more time to enter the soil profile.

### Methods of Cultivating Genetically Altered Plants

An aspect of the disclosure includes methods of cultivating genetically altered plants with increased water use efficiency, including the steps of: (a) providing the genetically altered plant, wherein the plant or a part thereof includes one or more genetic alterations; and (b) cultivating the genetically altered plant under conditions wherein the one or more genetic alterations increase activity of a Photosystem II Subunit S (PsbS) protein as compared to a wild type (WT) plant without the one or more genetic alterations, and wherein the increased activity of the PsbS protein increases water use efficiency as compared to the WT plant grown under the same conditions. An additional embodiment of this aspect includes the conditions being reduced irrigation conditions. A further embodiment of this aspect includes the conditions being rain fed conditions. Yet another embodiment of this aspect includes the conditions being high density growth conditions. Still another embodiment of this aspect includes the conditions being mild salinity. In an additional embodiment of this aspect, the conditions are fertilized or providing additional nutrients. In a further embodiment of this aspect, the conditions are humid conditions or conditions resulting in wet leaf surfaces. In another embodiment of this present aspect, which may be combined with any of the preceding embodiments, the increased activity of the PsbS protein provides the genetically altered plant with a higher yield, an increased biomass, an increased growth rate, an increased tolerance of salinity, an increased ability to withstand salinity, an increased flow of nutrients to the roots, an increased availability of nutrients over time, an increased utilization of fertilizer, an increased utilization of nutrients, a decreased susceptibility to a plant disease requiring humid conditions and/or wet leaf surfaces for infection, a decreased susceptibility to infection by the plant disease, a reduced incidence of the plant disease, or a reduced incidence of infection by the plant disease as compared to the WT plant without the increased activity grown under the same conditions where the activity of the PsbS protein is increased.

A further embodiment of this aspect that can be combined with any of the preceding embodiments includes the genetically altered plant being provided in step (a) by planting a genetically altered seedling, a genetically altered plantlet, a genetically altered cutting, a genetically altered tuber, a genetically altered root, or a genetically altered seed to produce the genetically altered plant, or by grafting the genetically altered seedling, the genetically altered plantlet, or the genetically altered cutting to a root stock or a second plant to produce the genetically altered plant. In still another embodiment of this aspect that can be combined with any of the preceding embodiments, the genetically altered plant is cultivated in step (b) to produce harvestable seed and fruits or vegetatively produced harvested items. An additional embodiment of this aspect includes the harvestable seed and fruits being selected from seed, fruit, pods, grain, kernels, beans, and peas; and wherein the vegetatively produced harvested items are selected from tubers, rhizomes, buds, roots, cuttings, and leaves. A further embodiment of this aspect that can be combined with any of the preceding embodiments that has harvestable seed and fruit or vegetatively produced harvestable items further includes harvesting the harvestable seed, harvestable leaves, harvestable roots, harvestable cuttings, harvestable fruit, harvestable kernels, harvestable tubers, harvestable pods, harvestable peas, harvestable beans, and/or harvestable grain.

Still another embodiment of this aspect that can be combined with any of the preceding embodiments includes the increased activity of the PsbS protein providing the genetically altered plant with decreased stomatal conductance as compared to the WT plant without the increased activity grown under the same conditions where the activity of the PsbS protein is increased. In yet another embodiment of this aspect, the increased activity of the PsbS protein provides the genetically altered plant with substantially similar photosynthetic efficiency as compared to the WT plant grown under the same conditions where the activity of the PsbS protein is increased.

### Genetically Altered Plants

An additional aspect of the disclosure includes a genetically altered plant or part thereof including one or more genetic alterations that increase activity of a PsbS protein as compared to a WT plant without the one or more genetic alterations, wherein the genetically altered plant shows increased water use efficiency as compared to the WT plant grown under the same conditions. An additional embodiment of this aspect includes the conditions being selected from the group of reduced irrigation conditions, rain fed conditions, high density growth conditions, mild salinity, fertilized or providing additional nutrients, humid conditions, and conditions resulting in wet leaf surfaces. In yet another embodiment of this aspect, which may be combined with any of the preceding embodiments, the increased activity of the PsbS protein provides the genetically altered plant with a higher yield, an increased biomass, an increased growth rate, an increased tolerance of salinity, an increased ability to withstand salinity, an increased flow of nutrients to the roots, an increased availability of nutrients over time, an increased utilization of fertilizer, an increased utilization of nutrients, a decreased susceptibility to a plant disease requiring humid conditions and/or wet leaf surfaces for infection, a decreased susceptibility to infection by the plant disease, a reduced incidence of the plant disease, or a reduced incidence of infection by the plant disease as compared to the WT plant without the increased activity grown under the same conditions where the activity of the PsbS protein is increased. In still another embodiment of this present aspect, which may be combined with any of the preceding embodiments, the genetically altered plant does not include increased activity of zeaxanthin epoxidase (ZEP) protein, violaxanthin de-epoxidase (VDE) protein, or both, as compared to a WT plant. In a further embodiment of this present aspect, which may be combined with any of the preceding embodiments, the genetically altered plant does not include reduced activity of K⁺ efflux antiporter 3 (KEA3) as compared to a WT plant. In yet another embodiment of this aspect that can be combined with any of the preceding embodiments, the genetically altered plant does not include reduced activity of K+/H+ antiporters or K+ efflux antiporters localized to the chloroplast thylakoid membrane (e.g., KEA3).

In still another embodiment of this aspect that can be combined with any of the relevant preceding embodiments, the genetically altered plant further includes increased activity of a ZEP protein and increased activity of a VDE protein, wherein the increased activity of the PsbS protein is greater than the increased activity of the PsbS protein required for increased photosynthetic efficiency when each of the ZEP protein, the PsbS protein, and the VDE protein are overexpressed together. An additional embodiment of this aspect includes increased activity of the PsbS protein being due to an increased amount of the PsbS protein. In yet another embodiment of this aspect, the increased amount of the PsbS protein is at least 10% greater, at least 11% greater, at least 12% greater, at least 13% greater, at least 14% greater, at least 15% greater, at least 16% greater, at least 17% greater, at least 18% greater, at least 19% greater, at least 20% greater, at least 21% greater, at least 22% greater, at least 23% greater, at least 24% greater, at least 25% greater, at least 26% greater, at least 27% greater, at least 28% greater, at least 29% greater, at least 30% greater, at least 31% greater, at least 32% greater, at least 33% greater, at least 34% greater, at least 35% greater, at least 36% greater, at least 37% greater, at least 38% greater, at least 39% greater, at least 40% greater, at least 41% greater, at least 42% greater, at least 43% greater, at least 44% greater, at least 45% greater, at least 46% greater, at least 47% greater, at least 48% greater, at least 49% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 110% greater, at least 120% greater, at least 130% greater, at least 140% greater, at least 150% greater, at least 160% greater, at least 170% greater, at least 180% greater, at least 190% greater, or at least 200% greater than the increased activity of the PsbS protein required for increased photosynthetic efficiency when each of the ZEP protein, the PsbS protein, and the VDE protein are overexpressed together. Still another embodiment of this aspect includes the increased amount of PsbS protein being no greater than 500%, no greater than 450%, no greater than 400%, no greater than 350%, no greater than 300%, no greater than 250%, no greater than 200%, no greater than 190%, no greater than 180%, no greater than 170%, no greater than 160%, no greater than 150%, no greater than 140%, no greater than 130%, no greater than 120%, no greater than 110%, no greater than 100%, no greater than 95%, no greater than 90%, no greater than 85%, no greater than 80%, no greater than 75%, no greater than 70%, no greater than 65%, no greater than 60%, no greater than 55%, or no greater than 50% of the increased activity of the PsbS protein required for increased photosynthetic efficiency when each of the ZEP protein, the PsbS protein, and the VDE protein are overexpressed together. Another embodiment of this aspect includes the increased amount of PsbS, the increased amount of ZEP, and the increased amount of VDE being increased relative to an amount of endogenous PsbS protein, an amount of endogenous ZEP protein, and an amount of endogenous VDE protein, and wherein the increased amount of PsbS is 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 55% or more, 60% or more, 65% or more, 70% or more, 75% or more, 80% or more, 85% or more, 90% or more, 95% or more, 100% or more, 110% or more, 120% or more, 130% or more, 140% or more, 150% or more, 160% or more, 170% or more, 180% or more, 190% or more, or 200% or more increased relative to the amount of endogenous PsbS protein, the increased amount of ZEP is 90% increased relative to the amount of endogenous ZEP protein, and the increased amount of VDE is 80% increased relative to the amount of endogenous VDE protein. In a further embodiment of this aspect, the increased amount of PsbS, the increased amount of ZEP, and the increased amount of VDE is increased relative to an amount of endogenous PsbS protein, an amount of endogenous ZEP protein, and an amount of endogenous VDE protein, and wherein the increased amount of PsbS is 12% increased relative to the amount of endogenous PsbS protein, the increased amount of ZEP is 90% increased relative to the amount of endogenous ZEP protein, and the increased amount of VDE is 80% increased relative to the amount of endogenous VDE protein. In still another embodiment of this aspect, the increased amount of PsbS, the increased amount of ZEP, and the increased amount of VDE is increased relative to an amount of endogenous PsbS protein, an amount of endogenous ZEP protein, and an amount of endogenous VDE protein, and wherein the increased amount of PsbS is 24% increased relative to the amount of endogenous PsbS protein, the increased amount of ZEP is 90% increased relative to the amount of endogenous ZEP protein, and the increased amount of VDE is 80% increased relative to the amount of endogenous VDE protein. In yet another embodiment of this aspect, which may be combined with any of the preceding embodiments that has increased activity of a ZEP protein and increased activity of a VDE protein, the increased activity of the PsbS, VDE, and ZEP proteins provides the genetically altered plant with increased photosynthetic efficiency under non-steady state conditions as compared to the WT plant without the increased activity grown under the same conditions where the activity of the PsbS, VDE, and ZEP proteins is increased.

Still another embodiment of this present aspect, which may be combined with any of the preceding embodiments, includes the genetically altered plant being selected from the group of cowpea (e.g., black-eyed pea, catjang, yardlong bean, Vigna unguiculata), soybean (e.g., Glycine max, Glycine soja), cassava (e.g., manioc, yucca, Manihot esculenta), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), barley (e.g., Hordeum vulgare), corn (e.g., maize, Zea mays), sorghum (e.g., Sorghum bicolor), rice (e.g., indica rice, japonica rice, aromatic rice, glutinous rice, Oryza sativa, Oryza glaberrima), cotton (e.g., Gossypium hirsutum, Gossypium barbadense, Gossypium arboretum, Gossypium herbaceum), rubber (e.g., Hevea brasiliensis), sugarcane (e.g., Saccharum officinarum, Saccharum spp.), eucalyptus (e.g., Eucalyptus spp.), willow (e.g., Salix spp.), birch (e.g., Betula spp.), beech (e.g., Fagus spp.), poplar (e.g., hybrid poplar, Populus trichocarpa, Populus tremula, Populus alba, Populus spp.), chestnut (e.g., Castanea spp.), citrus (e.g., lemon, lime, orange, grapefruit, pomelo, citron, trifoliate orange, bergamot orange, bitter orange, blood orange, satsuma, clementine, mandarin, yuzu, finger lime, kaffir lime, kumquat, Citrus clementina, Citrus sinensis, Citrus trifoliata, Citrus japonica, Citrus maxima, Citrus australasica, Citrus reticulata, Citrus aurantifolia, Citrus hystrix, Citrus x paradisi, Citrus x clementina, Citrus spp.), avocado (e.g., Persea americana), apple (e.g., Malus pumila, Malus x domestica, Pyrus malus), pear (e.g., Pyrus communis, Pyrus xbretschneideri, Pyrus pyrifolia, Pyrus sinkiangensis, Pyrus pashia, Pyrus spp.), cherry (e.g., Prunus avium Prunus cerasus), peach (e.g., Prunus persica), plum (e.g., Mirabelle, greengage, damson, Prunus domestica, Prunus salicina, Prunus mume), apricot (e.g., Prunus armeniaca, Prunus brigantine, Prunus mandshurica), fig (e.g., Ficus carica), olive (e.g., Olea europaea), almond (e.g., Prunus dulcis, Prunus amygdalus), pistachio (e.g., Pistacia vera), walnut (e.g., Persian walnut, English walnut, black walnut, Juglans regia, Juglans nigra, Juglans cinerea, Juglans californica), hazelnut (e.g., Corylus avellana, Corylus spp.), pecan (e.g., Carya illinoinensis), tomato (e.g., Solanum lycopersicum), eggplant (e.g., Solanum melongena), potato (e.g., russet potatoes, yellow potatoes, red potatoes, Solanum tuberosum), or alfalfa (e.g., Lucerne, Medicago sativa). An additional embodiment of this aspect includes the genetically altered plant not being rice or Arabidopsis (e.g., Arabidopsis thaliana).

Still another embodiment of this aspect that can be combined with any of the preceding embodiments includes the increased activity of the PsbS protein providing the genetically altered plant with decreased stomatal conductance as compared to the WT plant without the increased activity grown under the same conditions where the activity of the PsbS protein is increased. In yet another embodiment of this aspect, which may be combined with any of the preceding embodiments that does not have increased activity of a ZEP protein and increased activity of a VDE protein, the increased activity of the PsbS protein provides the genetically altered plant with substantially similar photosynthetic efficiency as compared to the WT plant grown under the same conditions where the activity of the PsbS protein is increased.

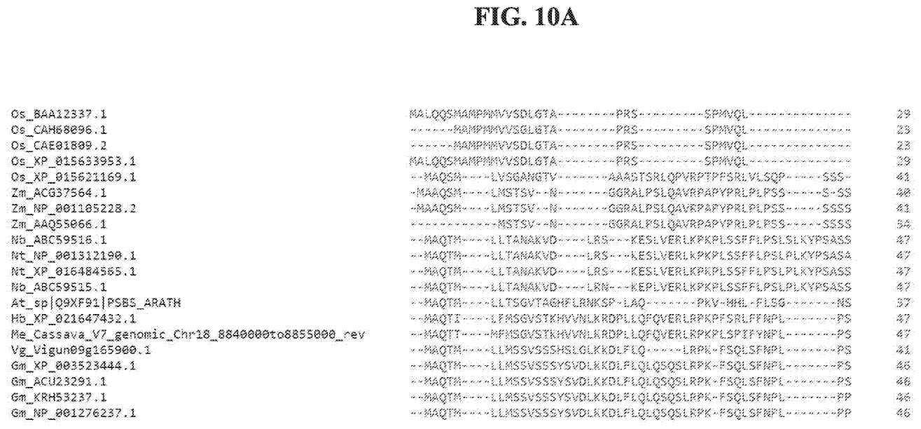

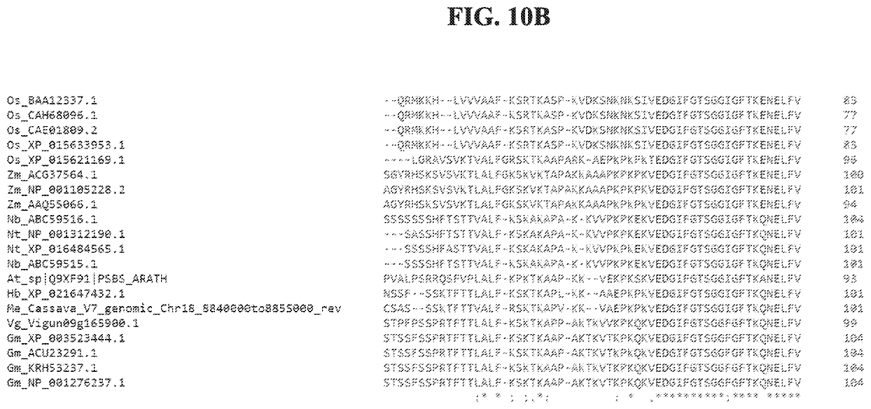

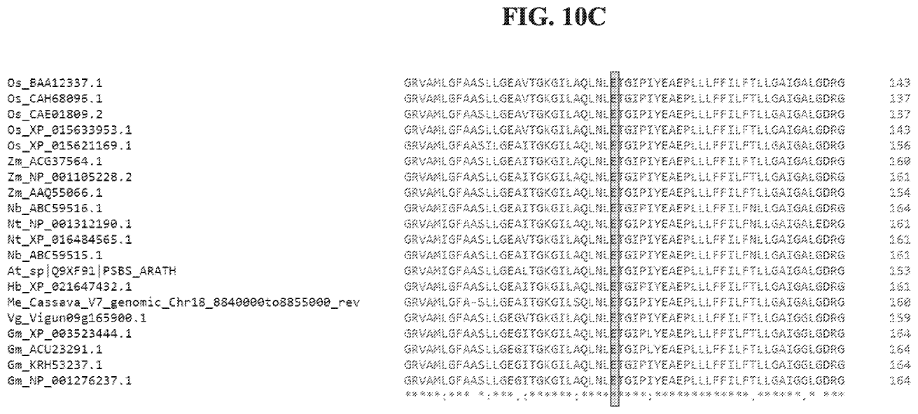

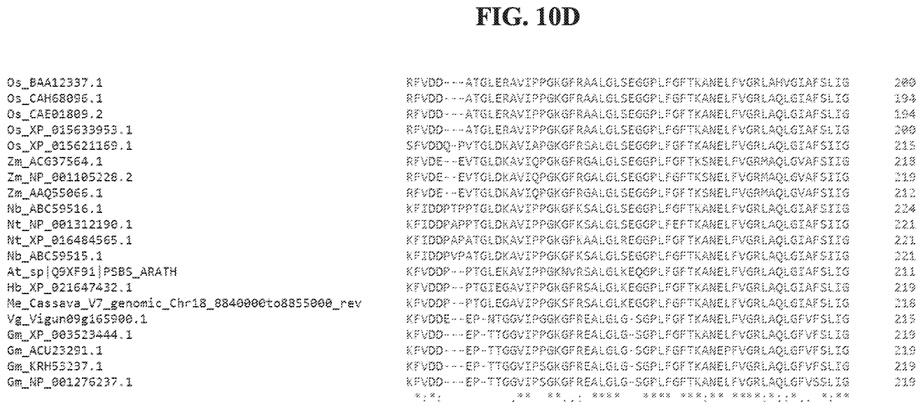

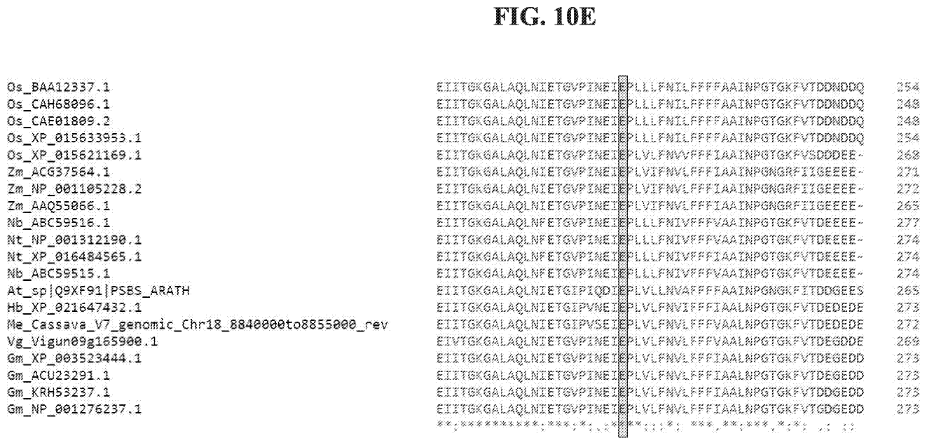

In a further embodiment of this aspect that can be combined with any of the preceding embodiments, increased activity is increased expression. An additional embodiment of this aspect includes the increased expression being due to expression of a heterologous PsbS protein. A further embodiment of this aspect includes the heterologous PsbS protein including at least 70% sequence identity to, at least 75% sequence identity to, at least 80% sequence identity to, at least 85% sequence identity to, at least 90% sequence identity to, at least 95% sequence identity to, or at least 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184. Yet another embodiment of this aspect includes the heterologous PsbS protein being selected from the group of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184. A further embodiment of this aspect that can be combined with any of the preceding embodiments that has a heterologous PsbS protein includes the heterologous PsbS protein containing a glutamate at a position corresponding to amino acid 149 of reference sequence SEQ ID NO: 21 and a glutamate at a position corresponding to amino acid 255 of reference sequence SEQ ID NO: 21. When the PsbS protein is expressed, it localizes to the chloroplast thylakoid membrane, and these two glutamates are located in short loop sequences that connect transmembrane domains. Both of these short loop sequences are located on the chloroplast lumen. The two glutamates are thought to act as pH sensors; by sensing the acidification of the chloroplast lumen, the glutamates allow PsbS to function as an excess light sensor (Li et al., J. Biol. Chem., (2004), 279 (22), 22866-22874). FIGS. 10A-10E show an alignment of exemplary PsbS protein sequences, and SEQ ID NO: 21 is a consensus protein sequence generated from the alignment. Still another embodiment of this aspect that can be combined with any of the preceding embodiments that has a heterologous PsbS protein includes the heterologous PsbS protein being encoded by a first nucleic acid and the first nucleic acid being operably linked to a second nucleic acid including a promoter. An additional embodiment of this aspect includes the promoter being selected from the group of a CaMV35S promoter, a ubiquitin promoter, a Rbcs1A promoter, a GAPA-1 promoter, a FBA2 promoter, and any combination thereof. Still another embodiment of this aspect that can be combined with any of the preceding embodiments that has a nucleic acid sequence includes the first nucleic acid sequence and the second nucleic acid sequence being stably integrated into a nuclear genome of the plant or into a chloroplast genome of the plant. A further embodiment of this aspect includes the increased expression being due to overexpression of an endogenous PsbS protein. An additional embodiment of this aspect includes overexpression of the endogenous PsbS protein being achieved using a gene editing technique to introduce the one or more genetic alterations that increase the activity of the endogenous PsbS protein. Still another embodiment of this aspect includes the gene editing technique being selected from the group of transcription activator-like effector nuclease (TALEN) gene editing techniques, clustered Regularly Interspaced Short Palindromic Repeat (CRISPR/Cas) gene editing techniques, or zinc-finger nuclease (ZFN) gene editing techniques. Still another embodiment of this aspect that can be combined with any of the preceding embodiments that has a gene editing technique includes the one or more genetic alterations that increase the activity of the endogenous PsbS protein are selected from the group of inactivating a repressor element that represses expression of the endogenous PsbS protein, removing a repressor element that represses expression of the endogenous PsbS protein, modulating the methylation state of a repressor element that represses expression of the endogenous PsbS protein, activating an enhancer element that increases expression of the endogenous PsbS protein, adding an enhancer element that increases expression of the endogenous PsbS protein, modulating the methylation state of an enhancer element that increases expression of the endogenous PsbS protein, adding a transcriptional activator recruiting or binding element that activates expression of the endogenous PsbS protein, replacing the endogenous promoter with an overexpression promoter that directs expression of the endogenous PsbS protein, modulating the methylation state of the endogenous promoter; modulating the methylation state of the endogenous PsbS coding sequence; adding elements that stabilize an endogenous PsbS mRNA, removing elements that destabilize the endogenous PsbS mRNA, modifying a PsbS coding sequence to increase stability of the PsbS protein, or modifying a PsbS coding sequence to increase activity of the PsbS protein. A further embodiment of this aspect that can be combined with any of the preceding embodiments that has a gene editing technique includes the one or more genetic alterations being epigenetic modifications.

In still another embodiment of this aspect that can be combined with any of the preceding embodiments, the PsbS protein is localized to a thylakoid membrane of at least one chloroplast within a cell of the genetically altered plant. An additional embodiment of this aspect includes the cell being a chloroplast containing leaf cell. A further embodiment of this aspect includes the cell being a mesophyll cell or a guard cell. In yet another embodiment of this aspect that can be combined with any of the preceding embodiments that has localization of the PsbS protein, the PsbS protein is expressed in at least 70% of the cells.

Yet another embodiment of this aspect that can be combined with any of the preceding embodiments with respect to seeds includes a genetically altered seed produced from the genetically altered plant of any one of the preceding embodiments. Still another embodiment of this aspect that can be combined with any of the preceding embodiments with respect to plant part includes the plant part being a leaf, a stem, a root, a flower, a seed, a kernel, a grain, a pod, a bean, a pea, a fruit, a chloroplast, a cell, or a portion thereof and the genetically altered plant part including the one or more genetic alterations. A further embodiment of this aspect includes the plant part being a fruit, a kernel, a grain, a pod, a bean, or a pea. Still another embodiment of this aspect that can be combined with any of the preceding embodiments with respect to pollen grain or ovules includes a genetically altered pollen grain or a genetically altered ovule of the plant of any one of the preceding embodiments, wherein the genetically altered pollen grain or the genetically altered ovule includes the one or more genetic alterations. A further embodiment of this aspect that can be combined with any of the preceding embodiments includes a genetically altered protoplast produced from the genetically altered plant of any of the preceding embodiments, wherein the genetically altered protoplast includes the one or more genetic alterations. An additional embodiment of this aspect that can be combined with any of the preceding embodiments includes a genetically altered tissue culture produced from protoplasts or cells from the genetically altered plant of any one of the preceding embodiments, wherein the cells or protoplasts are produced from a plant part selected from the group of leaf, leaf mesophyll cell, anther, pistil, stem, petiole, root, root tip, tuber, fruit, seed, kernel, grain, flower, cotyledon, hypocotyl, embryo, or meristematic cell, wherein the genetically altered tissue culture includes the one or more genetic alterations. An additional embodiment of this aspect includes a genetically altered plant regenerated from the genetically altered tissue culture that includes the one or more genetic alterations. In still another embodiment of this aspect, the genetically altered plant regenerated from the genetically altered tissue culture has all the physiological and morphological characteristics of the genetically altered plant produced from the method of any one of the preceding embodiments.

### Methods of Cultivating and Producing Genetically Altered Plants

An additional aspect of the disclosure includes methods of cultivating the genetically altered plant of any of the preceding embodiments including the steps of (a) planting a genetically altered seedling, a genetically altered plantlet, a genetically altered cutting, a genetically altered tuber, a genetically altered root, or a genetically altered seed to produce the genetically altered plant, or by grafting the genetically altered seedling, the genetically altered plantlet, or the genetically altered cutting to a root stock or a second plant to produce the genetically altered plant; (b) cultivating the genetically altered plant to produce harvestable seed and fruits or vegetatively produced harvested items, wherein the harvestable seed and fruits is selected from seed, fruit, pods, grain, kernels, beans, and peas, and wherein the vegetatively produced harvested items are selected from tubers, rhizomes, buds, roots, cuttings, and leaves; and (c) harvesting the harvestable seed, harvestable leaves, harvestable roots, harvestable cuttings, harvestable fruit, harvestable kernels, harvestable tubers, harvestable pods, harvestable peas, harvestable beans, and/or harvestable grain.

An additional aspect of the disclosure includes methods of producing the genetically altered plant of any of the preceding embodiments including the steps of (a) transforming a plant cell, tissue, or other explant with a vector including a first nucleic acid sequence encoding a PsbS protein operably linked to a second nucleic acid sequence encoding a promoter; (b) selecting successful transformation events by means of a selection agent, marker-assisted selection, or selective media; (c) regenerating the transformed cell, tissue, or other explant into a genetically altered plantlet; and (d) growing the genetically altered plantlet into a genetically altered plant with increased activity of a PsbS protein as compared to an untransformed WT plant. An additional embodiment of this aspect further includes identifying successful introduction of the one or more genetic alterations by screening or selecting the plant cell, tissue, or other explant prior to step (b); screening or selecting plantlets between step (b) and (c); or screening or selecting plants after step (c). In yet another embodiment of this aspect, which may be combined with any of the preceding embodiments, transformation is done using a transformation method selected from the group of particle bombardment (i.e., biolistics, gene gun), Agrobacterium-mediated transformation, Rhizobium-mediated transformation, or protoplast transfection or transformation. In still another embodiment of this aspect, which may be combined with any of the preceding embodiments, the vector is pEG100. A further embodiment of this aspect includes the PsbS protein including at least 70% sequence identity to, at least 75% sequence identity to, at least 80% sequence identity to, at least 85% sequence identity to, at least 90% sequence identity to, at least 95% sequence identity to, or at least 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184. Yet another embodiment of this aspect includes the PsbS protein being selected from the group of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184. A further embodiment of this aspect that can be combined with any of the preceding embodiments includes the PsbS protein containing a glutamate at a position corresponding to amino acid 149 of reference sequence SEQ ID NO: 21 and a glutamate at a position corresponding to amino acid 255 of reference sequence SEQ ID NO: 21. Still another embodiment of this aspect that can be combined with any of the preceding embodiments includes the heterologous PsbS protein being encoded by a first nucleic acid and the first nucleic acid being operably linked to a second nucleic acid including a promoter. An additional embodiment of this aspect includes the promoter being selected from the group of a CaMV35S promoter, a ubiquitin promoter, a Rbcs1A promoter, a GAPA-1 promoter, a FBA2 promoter, and any combination thereof. Still another embodiment of this aspect that can be combined with any of the preceding embodiments that has a nucleic acid sequence includes the first nucleic acid sequence and the second nucleic acid sequence being stably integrated into a nuclear genome of the plant or into a chloroplast genome of the plant.

A further aspect of the disclosure includes methods of producing the genetically altered plant of any of the preceding embodiments, including the steps of (a) transforming a plant cell, tissue, or other explant with one or more gene editing components that target a nuclear genome sequence operably linked to an endogenous PsbS protein; (b) selecting successful transformation events by means of a screening technology, an enriching technology, a selection agent, marker-assisted selection, or selective media; (c) regenerating the transformed cell, tissue, or other explant into a genetically altered plantlet; and (d) growing the genetically altered plantlet into a genetically altered plant with increased activity of a PsbS protein as compared to an untransformed WT plant. An additional embodiment of this aspect includes the one or more gene editing components including a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

### Molecular Biological Methods to Produce Genetically Altered Plants and Plant Cells

One embodiment of the present invention provides a genetically altered plant or plant cell containing one or more genetic alterations, which increase activity of a PsbS protein as compared to a WT plant without the one or more genetic alterations, and the increased activity increases water use efficiency as compared to the WT plant grown under the same conditions. For example, the present disclosure provides plants with increased activity of a PsbS protein due to expression of a heterologous PsbS protein. In addition, the present disclosure provides plants with increased activity of a PsbS protein due to overexpression of an endogenous PsbS protein.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); and Wang, et al. Acta Hort. 461:401-408 (1998). The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the inventions disclosed herein. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618) and rice (Shimamoto et al., Nature, (1989) 338, 274-276; Datta et al., Bio/Technology, (1990) 8, 736-740) and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present invention can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in nuclear DNA or as modifications to an endogenous gene or promoter. Plants including the genetic alteration(s) in accordance with the invention include plants including, or derived from, root stocks of plants including the genetic alteration(s) of the invention, e.g., fruit trees or ornamental plants. Hence, any non-transgenic grafted plant parts inserted on a transformed plant or plant part are included in the invention.

Introduced genetic elements, whether in an expression vector or expression cassette, which result in the expression of an introduced gene, will typically utilize a plant-expressible promoter. A ‘plant-expressible promoter’ as used herein refers to a promoter that ensures expression of the genetic alteration(s) of the invention in a plant cell. Examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294; Kay et al., Science, (1987) 236, 4805) and CabbB JI (Hull and Howell, Virology, (1987) 86, 482-493); promoters from the ubiquitin family (e.g., the maize ubiquitin promoter of Christensen et al., Plant Mol Biol, (1992) 18, 675-689, or the Arabidopsis UBQ10 promoter of Norris et al. Plant Mol. Biol. (1993) 21, 895-906), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the Cassava vein mosaic virus (WO 97/48819, Verdaguer et al. (Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the F and 2′ genes, respectively, of the T DNA (Velten et al., EMBO J, (1984) 3, 2723 2730).

Alternatively, a plant-expressible promoter can be a tissue-specific promoter, i.e., a promoter directing a higher level of expression in some cells or tissues of the plant, e.g., in leaf mesophyll cells. In preferred embodiments, leaf mesophyll specific promoters or leaf guard cell specific promoters are used. Non-limiting examples include the leaf specific Rbcs1A promoter (A. thaliana RuBisCO small subunit 315 1A (AT1G67090) promoter), GAPA-1 promoter (A. thaliana Glyceraldehyde 3-phosphate dehydrogenase 316 A subunit 1 (AT3G26650) promoter), and FBA2 promoter (A. thaliana Fructose-bisphosphate aldolase 2 317 (AT4G38970) promoter) (Kromdijk et al., Science, 2016). Further non-limiting examples include the leaf mesophyll specific FBPase promoter (Peleget al., Plant J, 2007), the maize or rice rbcS promoter (Nomura et al., Plant Mol Biol, 2000), the leaf guard cell specific Arabidopsis KAT1 promoter (Nakamura et al., Plant Phys, 1995), the Arabidopsis Myrosinase-Thioglucoside glucohydrolase 1 (TGG1) promoter (Husebye et al., Plant Phys, 2002), the Arabidopsis rhal promoter (Terryn et al., Plant Cell, 1993), the Arabidopsis AtCHX20 promoter (Padmanaban et al., Plant Phys, 2007), the Arabidopsis HIC (High carbon dioxide) promoter (Gray et al., Nature, 2000), the Arabidopsis CYTOCHROME P450 86A2 (CYP86A2) mono-oxygenase promoter (pCYP) (Francia et al., Plant Signal & Behav, 2008; Galbiati et al., The Plant Journal, 2008), the potato ADP-glucose pyrophosphorylase (AGPase) promoter (Muller-Rober et al., The Plant Cell 1994), the grape R2R3 MYB60 transcription factor promoter (Galbiati et al., BMC Plant Bio, 2011), the Arabidopsis AtMYB60 promoter (Cominelli et al., Current Bio, 2005; Cominelli et al., BMC Plant Bio, 2011), the Arabidopsis At1g22690-promoter (pGC1) (Yang et al., Plant Methods, 2008), and the Arabidopsis AtMYB 61 promoter (Liang et al., Curr Biol, 2005). These plant promoters can be combined with enhancer elements, they can be combined with minimal promoter elements, or can include repeated elements to ensure the expression profile desired.

In some embodiments, genetic elements to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present invention can be inserted in host cell DNA so that the inserted gene part is upstream (i.e., 5′) of suitable 3′ end transcription regulation signals (e.g., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835 845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981 6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (e.g., detectable mRNA transcript or protein is produced) throughout subsequent plant generations. Stable integration into and/or editing of the nuclear genome can be accomplished by any known method in the art (e.g., microparticle bombardment, Agrobacterium-mediated transformation, CRISPR/Cas9, electroporation of protoplasts, microinjection, etc.).

Further, any epigenetic methodology known in the art to alter expression of the cellular DNA (e.g., genomic DNA and organelle DNA) can be used in practicing the inventions disclosed herein (e.g., genetic alterations include epigenetic modifications). One type of epigenetic modification well known in the art is DNA methylation (and hydroxymethylation). For example, the methylation state of a gene promoter can affect the expression of a gene (as described, for example, in Ikeda et al., Plant Cell Physiol., (2007), 48(2), 205-220). The methylation state of the promoter can be changed using CRISPR-Cas SunTag systems (as described, for example, in Huang et al., Genome Biol., (2017) 18(1), 176 and Papikian et al., Nat. Commun., (2019) 10(1), 729) or demethylase zinc finger fusion systems (as described, for example, in Gallego-Bartolomé et al., PNAS, (2018) 115(9), E2125-E2134). Using these methods, the methylation of the endogenous PsbS promoter can be changed, and this changed methylation state can result in altered (e.g., increased) PsbS expression. Other types of epigenetic modifications well known in the art are histone modifications (e.g., acetylation, methylation, sumoylation, ubiquitination), which result in chromatin remodeling. Chromatin remodeling can increase gene expression, for example via regulation of transcription elongation efficiency (Nonogaki, Front. Plant. Sci., (2014) 5, 233), and can also result in gene silencing (Xu and Shen, Curr. Biol., (2008) 18(24), 1966-1971). Trithorax and Polycomb group proteins (Kleinmanns and Schubert, Biological Chemistry, (2014) 395(11), 1291-1300) as well as SWI/SNF proteins (Ojolo et al., Front. Plant. Sci., (2018) 9, 1232) have been identified as important components in histone methylation processes in plants. Epigenetic modifications, in particular DNA methylomes, in plants have been shown to be heritable over multiple generations, and are therefore well-suited for developing plant lines. One caveat is that the heritability of methylomes requires sexual reproduction: a study done to determine why oil palm trees had decreased yield revealed that the clonal reproduction of these trees had hindered the re-establishment of DNA methylation marks (Ong-Abdullah et al., Nature, (2015) 525, 533-537).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the terms “overexpression” and “upregulation” refer to increased expression (e.g., of mRNA, polypeptides, etc.) relative to expression in a wild type organism (e.g., plant) as a result of genetic modification. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is overexpressed. In some embodiments, an exogenous gene is overexpressed by virtue of being expressed. Overexpression of a gene in plants can be achieved through any known method in the art, including but not limited to, the use of constitutive promoters, inducible promoters, high expression promoters, enhancers, transcriptional and/or translational regulatory sequences, codon optimization, modified transcription factors, and/or mutant or modified genes that control expression of the gene to be overexpressed.

Where a recombinant nucleic acid is intended for expression, cloning, or replication of a particular sequence, DNA constructs prepared for introduction into a host cell will typically include a replication system (e.g. vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies of the invention disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present invention. One of skill in the art will recognize that any relevant markers available can be utilized in practicing the inventions disclosed herein.

Screening and molecular analysis of recombinant strains of the present invention can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to the subject invention. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present invention can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty:2, Nucleic match: 1, Nucleic mismatch −3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present invention can be introduced by any means known to the art which is appropriate for the particular type of cell, including without limitation, transformation, lipofection, electroporation or any other methodology known by those skilled in the art.

Marker-Assisted Breeding of Plants with Increased WUE Activity

Still another aspect of the disclosure includes methods of identifying genetic markers associated with increased PsbS protein activity in a plant, including the steps of (a) screening a population of plants from the same species or closely related species for PsbS protein activity; (b) identifying a subset of plants from the population with higher levels of PsbS activity as compared to the other plants in the population; and (c) identifying genetic markers associated with increased PsbS activity in the subset of plants. An additional embodiment of this aspect includes the increased PsbS protein activity being due to increased expression of a PsbS mRNA and the screening in step (a) being assaying levels of the PsbS mRNA, optionally using a method selected from the group of RNA-Seq, microarray, Northern blot, or qRT-PCR. A further embodiment of this aspect includes the increased PsbS protein activity being due to increased amount of the PsbS protein and the screening in step (a) being assaying levels of the PsbS protein, optionally using a method selected from the group of Western blot, ELISA, immunoprecipitation, HPLC, or LC/MS.

Yet another aspect of the disclosure includes methods of producing a plant with increased WUE activity and a second desired trait, including the steps of (a) providing a first plant including a genetic marker associated with increased PsbS activity and a second plant including the second desired trait; (b) crossing the first plant with the second plant to create a population of progeny plants; and (c) selecting the plant with increased WUE activity and the second desired trait using the genetic marker associated with increased PsbS activity. An additional embodiment of this aspect includes the second desired trait being increased PsbS activity unlinked to the genetic marker associated with increased PsbS activity. An additional embodiment that can be combined with any of the preceding embodiments includes the plant being selected from the group of cowpea, soybean, cassava, wheat, barley, corn, sorghum, rice, cotton, sugarcane, eucalyptus, poplar, willow, orange, grapefruit, lemon, lime, avocado, cherry, peach, plum, apricot, nectarine, fig, olive, almond, pistachio, walnut, chestnut, hazelnut, pecan, tomato, eggplant, potato, and alfalfa.

### Plant Breeding Methods

Plant breeding begins with the analysis of the current germplasm, the definition of problems and weaknesses of the current germplasm, the establishment of program goals, and the definition of specific breeding objectives. The next step is the selection of germplasm that possess the traits to meet the program goals. The selected germplasm is crossed in order to recombine the desired traits and through selection, varieties or parent lines are developed. The goal is to combine in a single variety or hybrid an improved combination of desirable traits from the parental germplasm. These important traits may include higher yield, field performance, improved fruit and agronomic quality, resistance to biological stresses, such as diseases and pests, and tolerance to environmental stresses, such as drought and heat.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.). Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection. These processes, which lead to the final step of marketing and distribution, usually take five to ten years from the time the first cross or selection is made.

The choice of breeding or selection methods depends on the mode of plant reproduction, the heritability of the trait(s) being improved, and the type of cultivar used commercially (e.g., F₁ hybrid cultivar, inbred cultivar, etc.). For highly heritable traits, a choice of superior individual plants evaluated at a single location will be effective, whereas for traits with low heritability, selection should be based on mean values obtained from replicated evaluations of families of related plants. The complexity of inheritance also influences the choice of the breeding method. Backcross breeding is used to transfer one or a few genes for a highly heritable trait into a desirable cultivar (e.g., for breeding disease-resistant cultivars), while recurrent selection techniques are used for quantitatively inherited traits controlled by numerous genes, various recurrent selection techniques are used. Commonly used selection methods include pedigree selection, modified pedigree selection, mass selection, and recurrent selection.

Pedigree selection is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding (i.e., recurrent selection) may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Molecular markers, or “markers”, can also be used during the breeding process for the selection of qualitative traits. For example, markers closely linked to alleles or markers containing sequences within the actual alleles of interest can be used to select plants that contain the alleles of interest. The use of markers in the selection process is often called genetic marker enhanced selection or marker-assisted selection. Methods of performing marker analysis are generally known to those of skill in the art.

Mutation breeding may also be used to introduce new traits into plant varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development: Theory and Technique, Walter Fehr (1991), Agronomy Books, 1 (lib.dr.iastate.edu/agron_books/1).

The production of double haploids can also be used for the development of homozygous lines in a breeding program. Double haploids are produced by the doubling of a set of chromosomes from a heterozygous plant to produce a completely homozygous individual. For example, see Wan, et al., Theor. Appl. Genet., 77:889-892, 1989.

Additional non-limiting examples of breeding methods that may be used include, without limitation, those found in Principles of Plant Breeding, John Wiley and Son, pp. 115-161 (1960); Principles of Cultivar Development: Theory and Technique, Walter Fehr (1991), Agronomy Books, 1 (lib.dr.iastate.edu/agron_books/1), which are herewith incorporated by reference.

Having generally described this invention, the same will be better understood by reference to certain specific examples, which are included herein to further illustrate the invention and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1

### Manipulation of PsbS Expression in Nicotiana tabacum Plants Under Controlled and Field-Grown Conditions

The following example describes the development and validation of Nicotiana tabacum (N. tabacum) lines with altered PsbS expression grown under controlled and field-grown conditions. N. tabacum lines with increased or decreased PsbS expression were generated.

### Materials and Methods

Developing N. tabacum Lines with Altered PsbS Expression

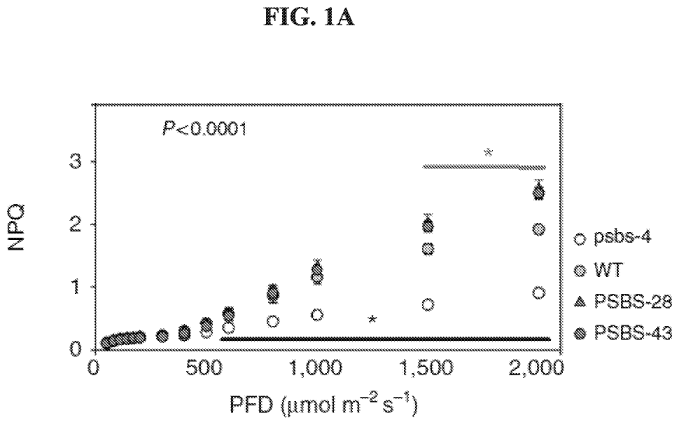

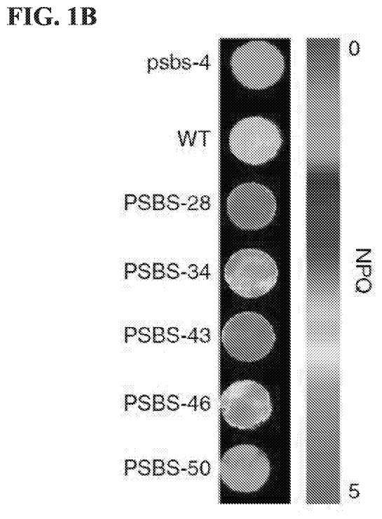

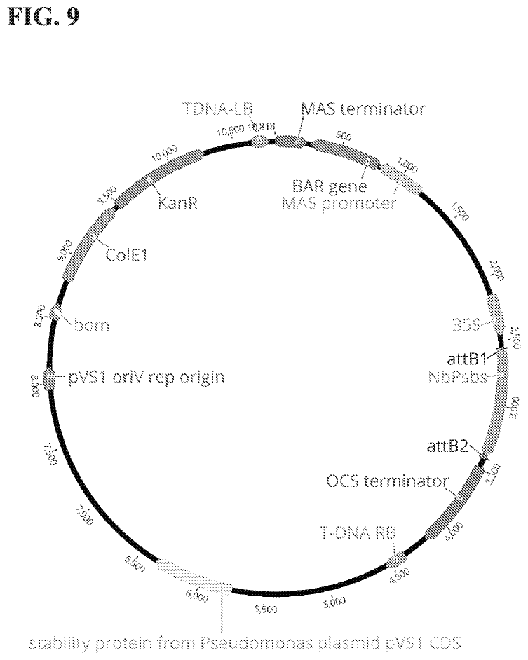

The N. benthamiana PsbS gene coding sequence (Uniprot—Q2LAHO_NICBE; e.g., encoding the protein of SEQ ID NO: 17 or SEQ ID NO: 18) was cloned between the cauliflower mosaic virus 35S and octopine synthase terminator in the pEARLYGATE 100 binary vector (pEG100-NbPsbS) (FIG. 9). N. tabacum cv. “Petite Havana” was transformed with pEG100-NbPsbS using the Agrobacterium tumefaciens-mediated protocol. Four independent, single-copy transformation events with increased NPQ amplitude (PSBS-28, PSBS-34, PSBS-43, and PSBS-46) were selected and selfed to obtain progeny homozygous for the transgene (FIGS. 1A and 1B). Copy number and homozygosity were assessed using digital droplet PCR (dd-PCR) as in Glowacka et al. (2016) Plant Cell Env 39, 908. In addition, two events exhibiting spontaneous partial silencing of PsbS expression (psbs-4 and PSBS-50) were selected. Spontaneous partial silencing through RNAi inhibition is a common occurrence when using genes from the same or a closely related species. Here, an N. benthamiana gene was used in N. tabacum.

### Seedling Propagation for Controlled Conditions

Seedlings were germinated on growing medium (LC1 Sunshine mix, Sun Gro Horticulture) in a controlled-environment cabinet (Environmental Growth Chambers) with photoperiod set to 12 h and temperature controlled at 23/18° C. (day/night). Five days after germination, seedlings were transplanted to 3.8-L pots and randomly positioned in a controlled-environment chamber (PGC20, Conviron) with photoperiod set to 16 h and air temperature controlled at 20/25° C. (day/night). Light intensity at leaf-level was controlled at 500 μmol m⁻² s⁻¹. Plants were watered and repositioned at random every 2 days until the fifth leaf was fully expanded.

### Seedling Propagation for Field-Grown Conditions

Homozygous seeds were sown in a greenhouse. Five days after germination, seedlings were propagated hydroponically for 2 weeks in floating trays (Transplant Tray GP009 6×12 cells, Speedling Inc.) filled with hydroponics growing medium (Pro-mix PGX, Premier Tech). The concentration of total dissolved solids in the solution was measured every 2 days with a handheld TDS meter (COM-100, HM Digital Inc.) and adjusted to 100 ppm by the addition of 20-10-20 water-soluble fertilizer (Jack's Professional, JR Peters Inc.). Five days after the transplant to trays, Etridiazole fungicide (Terramaster 4EC to a final concentration of 78 μL L⁻¹, Crompton Manufacturing Company Inc.) was added to the solution to protect the plants against root fungus disease in the field. Two applications of Mancozeb (Dithane Rainshield Fungicide at 1 g L⁻¹, Dow AgroSciences) were applied 6 and 9 days after transplant to prevent foliar fungus disease. On the same days, seedlings were sprayed with fermentation solids and solubles from Bacillus thuringiensis, (strain AM65-52, Gnatrol WDG Biological larvicide at 1 mL L⁻¹, Valent Biosciences Corp.) to reduce the greenhouse population of fungus gnats.

### Field-Grown Conditions

Seedlings were transplanted to an experimental field site. The field was prepared 2 weeks prior to transplant by rototilling, cultivation, and harrowing. At this time, chlorpyrifos (1.5 g m⁻² Lorsban 15 G Insecticide, Dow AgroSciences) was worked into the soil to suppress cutworm damage, sulfentrazone (29 μL m⁻² Spartan 4 F pre-emergence herbicide, FMC Agricultural Solutions) was applied to reduce the emergence of weeds and slow-release fertilizer (30.8 g m⁻² ESN Smart Nitrogen, Agrium US Inc.) was put down. After transplant, all seedlings were sprayed with thiamethoxam (7 mg/plant Platinum 75 SG insecticide, Syngenta Crop Protection) to prevent damage from insect herbivory, and 12 days after the field transplant, all plants were sprayed with fermentation solids, spores, and insecticidal toxins from Bacillus thuringiensis, (strain ABTS-351, DiPel Pro dry flowable biological insecticide, Valent Biosciences Corp.) to suppress tobacco hornworm. The field experiment was set up as an incomplete randomized block design with 12 blocks of 6×6 plants spaced 30 cm apart. Each block contained four rows of four plants per genotype in north-south (N-S) orientation, surrounded by one border row of WT. WT was present in all blocks (n=12), whereas the PsbS mutant lines were randomly assigned to six blocks (n=6). The blocks were positioned in 3 (N-S)×4 (E-W) rectangles with 75 cm spacing between blocks. The entire experiment was surrounded by two border rows of WT plants.

Light intensity (LI-190R quantum sensor, LI-COR) and air temperature (Model 109 temperature probe, Campbell Scientific) were measured nearby on the same field site and half-hourly averages were logged using a datalogger (CR1000, Campbell Scientific). Precipitation was measured at two locations close to the field using precipitation gauges (NOAH IV Precipitation Gauge, ETI Instrument Systems Inc.). Watering to restore field capacity was provided daily when needed through parallel drip irrigation lines with emitters every 30 cm (17 mm PC Drip Line #DL077, The Drip Store). mRNA Expression Analysis

Five leaf discs (total 2.9 cm²) from the youngest fully expanded leaf were collected for mRNA extraction. For plants grown under field-grown conditions, leaf discs were collected at 34, 37, 41, and 45 days after emergence. Leaf discs were isolated 2 hours (h) after the start of the photoperiod. mRNA was harvested using the NucleoSpin RNA/Protein kit (Macherey-Nagel GmbH & Co., REF740933). Then, the extracted mRNA was treated with DNase (Turbo DNA-free kit; Thermo Fisher Scientific, AM1907). Next, the mRNA was transcribed into cDNA using Superscript III First-Strand Synthesis System for RT-PCR (Thermo Fisher Scientific, 18089-051). Quantitative reverse transcription PCR (qRT-PCR) was used to quantify the amount of PsbS transcript expression relative to NtActin and NtTubulin.

### Protein Expression Analysis

Five leaf discs (total area of 2.9 cm²) from the youngest fully expanded leaf of five plants per genotype (when grown under controlled conditions) or four plants per genotype (when grown under field-grown conditions) were collected for protein extraction. Samples were isolated 2 h after the start of the photoperiod. Protein was harvested using the NucleoSpin RNA/Protein kit (Macherey-Nagel GmbH & Co., REF740933). Total protein concentration was quantified using a protein quantification assay (Macherey-Nagel GmbH & Co., REF740967.50). Samples containing 1 g total protein were separated by sodium dodecyl sulfate-polyacrylamide gel electrophoresis (SDS-PAGE) electrophoresis (Immobilon-P, Millipore, IPVH00010). Following separation, the samples were blotted to a membrane using semi-dry blotting (Trans-Blot SD, Bio-Rad). Then, the samples were labeled with primary antibodies raised against AtPsbS (AS09533, Agrisera) at a dilution of 1:2,000 and AtPsbO (AS06142-33, Agrisera) at a dilution of 1:20,000. Following primary antibody labeling, the samples were labeled with secondary antibody (W401B, Promega) at a dilution of 1:2,500. Last, the total protein in each labeled band was quantified using densitometry (ImageQuant LAS-4010, GE Healthcare Life Sciences) with ImageQuant TL software (Version 7.0 GE Healthcare Life Sciences). PsbS expression was normalized based on PsbO bands.

### NPQ Measurements in Plants Grown Under Controlled Conditions

NPQ measurements were performed using an open gas exchange system equipped with a 2-cm² leaf chamber and integrated modulated fluorometer (LI-COR, LI6400XT).

NPQ of chlorophyll fluorescence was determined assuming a Stern-Volmer quenching model according to the following equation: NPQ=Fₘ/Fₘ′−1. Block temperature was controlled at 25° C., CO₂ inside the cuvette was maintained at 380 μmol mol⁻¹, and leaf-to-air water VPD was controlled to 1.1-1.4 kPa. Leaves were clamped in the leaf cuvette and dark-adapted for 1 h, after which minimal (Fₒ) and maximal fluorescence (Fₘ) were measured to determine maximal efficiency of whole-chain electron transport. Subsequently, light intensity (100% red LEDs, λₚₑₐₖ 630 nm) was slowly increased from 0 to 50, 80, 110, 140, 170, 200, 300, 400, 500, 600, 800, 1,000, 1,500, and 2,000 μmol m⁻² s⁻¹. When steady state was reached, maximal fluorescence without dark adaptation (Fₘ′) was measured.

### NPQ Measurements in Plants Grown Under Field-Grown Conditions

Leaf discs were sampled pre-dawn from field-grown plants and stored in darkness in glass vials for up to 4 h until measurement. Humidity in the vials was maintained fully saturated by placing a piece of wet filter paper in each vial. Dark-adapted leaf discs were positioned on a piece of wet filter paper in a chlorophyll fluorescence imager to determine maximal fluorescence (Fₘ)(CFimager, Technologica). Subsequently, leaf discs were exposed to 15 min of 1000 μmol m² s⁻¹, after which maximal fluorescence without dark adaptation was determined (Fₘ′). NPQ was then determined according to the following equation: NPQ=Fₘ/Fₘ′−1.

### Results

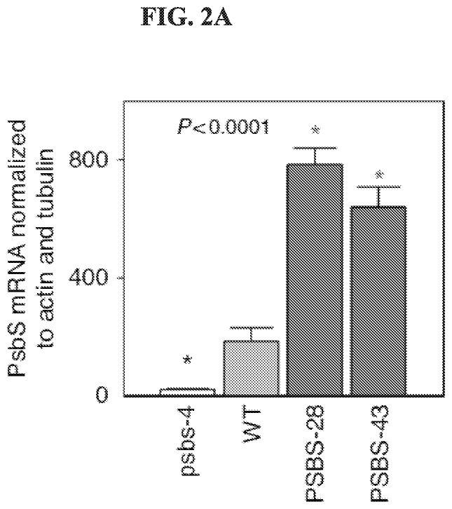

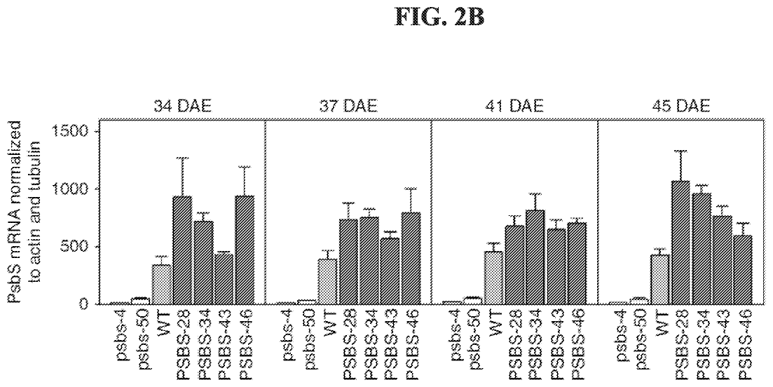

FIG. 2A shows PsbS mRNA expression in WT and N. tabacum plants with altered PsbS expression grown under controlled conditions. In particular, FIG. 2A shows PsbS expression increased 4.2-fold and 3.5-fold in the overexpression lines PSBS-28 and PSBS-43 respectively relative to WT plants grown under the same conditions. In contrast, PsbS expression decreased 10-fold in the partially silenced line psbs-4 relative to the WT samples (FIG. 2A).

FIG. 2B shows PsbS mRNA expression in WT and N. tabacum plants with altered PsbS expression under field-grown conditions. The overexpression lines PSBS-28, PSBS-34, PSBS-43, and PSBS-46 had elevated PsbS expression under field-grown conditions relative to WT, while the partially silenced lines psbs-4 and psbs-50 had decreased PsbS expression relative to WT (FIG. 2B).

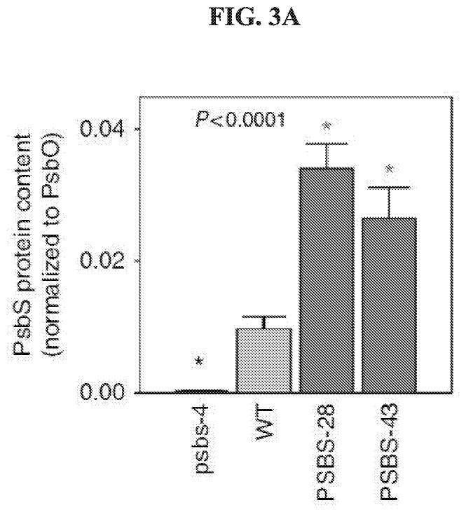

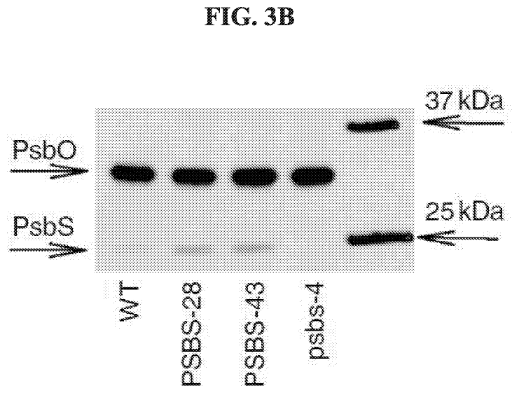

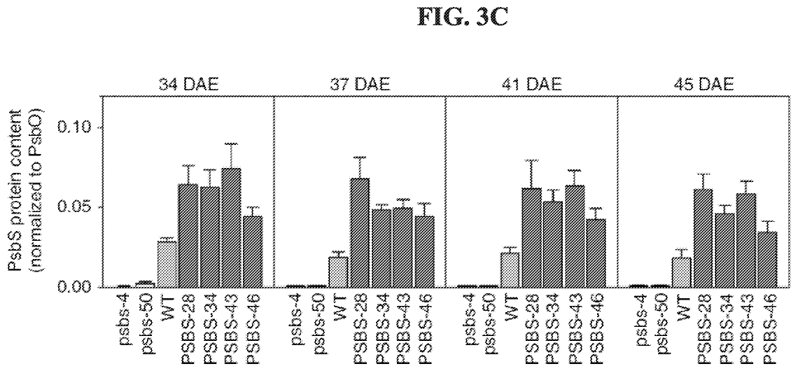

FIGS. 3A and 3B show PsbS protein expression normalized to PsbO protein expression in WT and mutant N. tabacum lines grown under controlled conditions. Specifically, PsbS protein expression was 2.7-fold and 3.5-fold higher in the overexpression lines PSBS-43 and PSBS-28 relative to WT (FIGS. 3A and 3B). PsbS protein was virtually absent in the partially silenced line psbs-4 (FIGS. 3A and 3B).

FIG. 3C shows PsbS protein expression normalized to PsbO protein expression in WT and mutant N. tabacum lines under field-grown conditions. FIG. 3C demonstrates that the overexpression lines PSBS-28, PSBS-34, PSBS-43, and PSBS-46 had elevated PsbS protein expression under field-grown conditions relative to WT (FIG. 3C). The partially silenced lines psbs-4 and psbs-50 had decreased PsbS expression relative to WT.

The results presented in FIGS. 2A-2B and FIGS. 3A-3C demonstrate that in the overexpression lines (PSBS-28, PSBS-34, PSBS-43, and PSBS-46), both mRNA and protein levels were increased relative to WT plants, while in the partially silenced lines (psbs-4 and psbs-50), both mRNA and protein levels were decreased relative to WT plants. These effects were seen regardless of growth conditions (i.e., both under controlled conditions and field-grown conditions).

### Example 2

### PsbS Expression Affects Water Use Efficiency in Plants Grown Under Controlled and Field-Grown Conditions

Example 1 detailed the development of N. tabacum lines with altered PsbS mRNA/protein expression. The following example describes the effect of altered PsbS expression levels in these lines on leaf-level instantaneous water use efficiency (iWUE) in plants grown under controlled and field-grown conditions.

### Materials and Methods

### Photosynthetic Gas Exchange Measurements in Plants Grown Under Controlled Conditions

Gas exchange measurements were performed using an open gas exchange system equipped with a 2-cm² leaf chamber and integrated modulated fluorometer (LI6400XT, LI-COR). All chlorophyll fluorescence measurements were performed using the multiphase flash routine.

To determine the light response of net CO₂ assimilation rate (Aₙ) and whole-chain photosynthetic electron transport, gas exchange and pulse amplitude-modulated chlorophyll fluorescence were measured at a range of light intensities. Block temperature was controlled at 25° C., CO₂ inside the cuvette was maintained at 380 μmol mol⁻¹, and leaf-to-air water vapor pressure deficit (VPD) was controlled to 1.1-1.4 kPa. Leaves were clamped in the leaf cuvette and dark-adapted for 1 h, after which minimal (Fₒ) and maximal fluorescence (Fₘ) were measured to determine maximal efficiency of whole-chain electron transport (Equation: Fᵥ/Fₘ=(Fₘ−Fₒ)/Fₘ).

Subsequently, light intensity (100% red LEDs, λₚₑₐₖ 630 nm) was slowly increased from 0 to 50, 80, 110, 140, 170, 200, 300, 400, 500, 600, 800, 1,000, 1,500, and 2,000 mol m⁻² s⁻¹. When steady state was reached, net CO₂ fixation rate (Aₙ), stomatal conductance (gₛ), and CO₂ in the intercellular airspaces within the leaf (Cᵢ), were logged. Instantaneous water use efficiency (iWUE) was determined by dividing net CO₂ assimilation by stomatal conductance (Aₙ/gₛ). Further, F′ and Fₘ′ were measured to estimate the operating efficiency of whole-chain electron transport (F_{q}′/Fₘ′=(Fₘ′−F′)/Fₘ′). Given that stomatal movements can include very long-term diurnal components, the routine was aimed at measuring only relatively short-term stomatal responses to changes in light intensity, and steady-state waiting times were kept between 10 and 20 minutes per step.

To evaluate the CO₂ response of A., leaves were allowed to reach steady state at a light intensity of 2,000 μmol m⁻² s⁻¹ (100% red LEDs, λₚₑₐₖ 630 nm), with block temperature controlled to 25° C. and CO₂ in the airstream set to 400 μmol mol⁻¹. Subsequently, CO₂ was varied from 400 to 300, 200, 100, 75, 400, 400, 500, 600, 700, 800, 1,000, 1,200, and 1,500 μmol mol⁻¹. When steady state was attained, Aₙ, gₛ, and Cᵢ were logged. V_{cmax} was determined from the response of Aₙ to chloroplastic CO₂ concentration (C_{c}) by fitting a biochemical model with temperature corrections to measurements. Jₘₐₓ was determined by fitting a non-rectangular hyperbola to light response curves of linear electron transport estimated from chlorophyll fluorescence. Stomatal limitation of Aₙ was computed using measurements at ambient CO₂ (Ca=380 μmol mol⁻¹) and saturating light intensity, and predicted values of Aₙ when stomata are not limiting.

### Photosynthetic Gas Exchange Measurements in Plants Grown Under Field-Grown Conditions

The response of photosynthetic gas exchange to light intensity was measured on the youngest fully expanded leaf of plants. Measurements were performed in four complete sets to account for random effects of North to South positioning of plants, and time of day. Leaves were clamped in the cuvette of an open gas exchange system (LI6400XT, LI-COR) and allowed to reach steady-state gas exchange at saturating light intensity of 2000 μmol m² s⁻¹, with block temperature set to 30° C. and CO₂ in the airstream controlled to 400 μmol mol⁻¹ and VPD between air and leaf kept below 1.5 kPa. Subsequently, light intensity was varied from 2,000 to 1,500, 1,000, 800, 600, 400, 300, 200, 170, 140, 110, 80, and 50 μmol m⁻² s⁻¹. Due to the limited window suitable for measuring gas exchange in field-grown trials, waiting time for steady state was kept between 5 and 10 min for these measurements. When steady state was reached, net assimilation rate (Aₙ), stomatal conductance (gₛ), and intercellular CO₂ (Cᵢ) were logged.

### Stomatal Density and Stomatal Complex Dimensions

Fresh leaf samples were taken from the youngest fully expanded leaf and mounted onto a microscope slide. Topographies of the adaxial and abaxial surfaces were measured using a surf explorer optical topometer (Nanofocus, Oberhausen). The 20x/0.60 objective lens was used for stomatal density quantification (Image J 1.51K, NIH). The 50x/0.80 objective lens was used for measurements of stomatal complex dimensions.

### Results

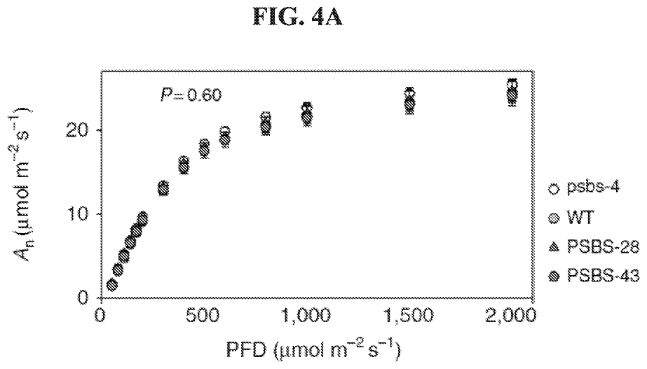

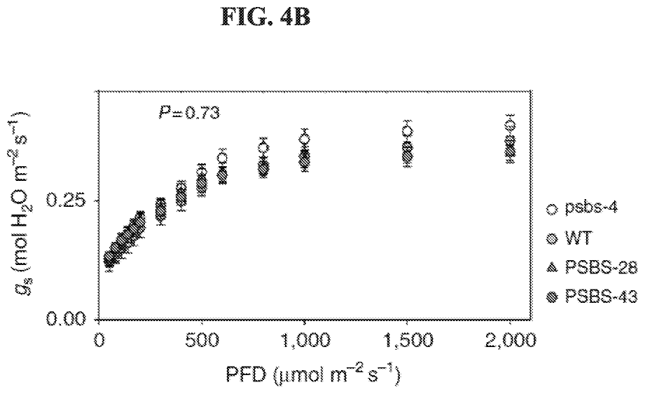

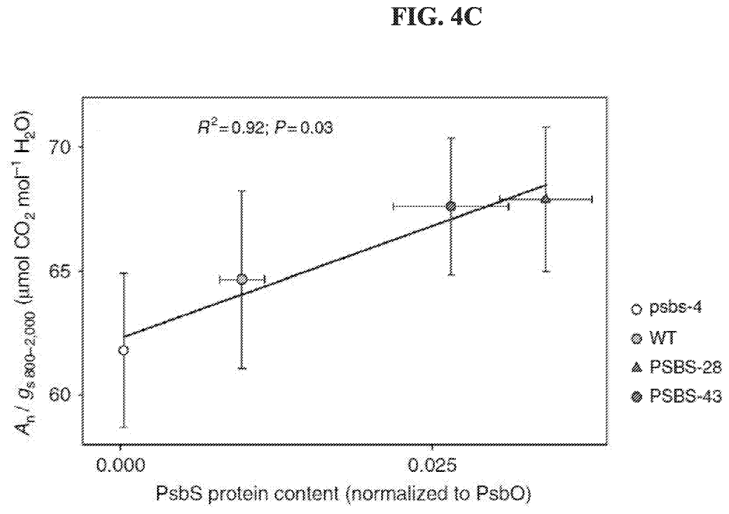

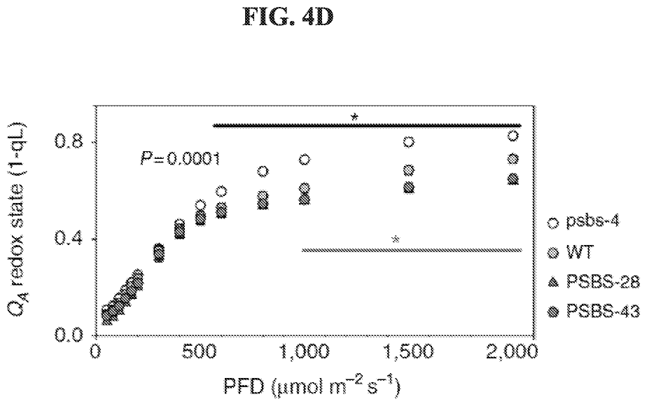

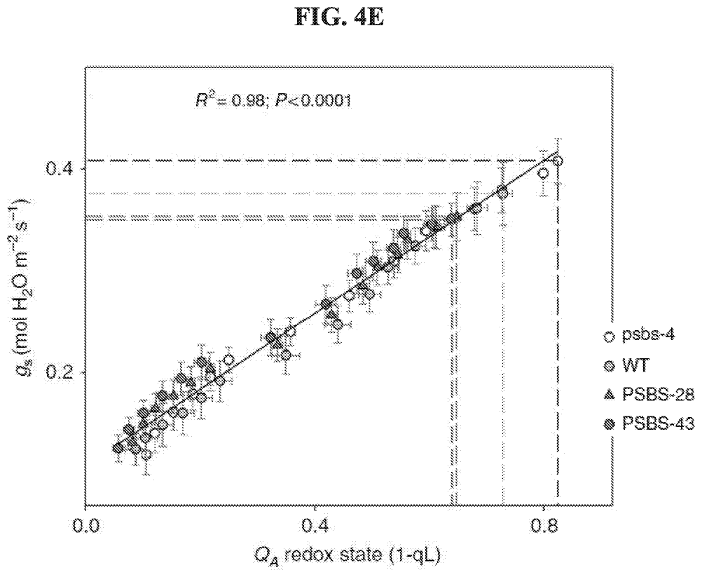

FIGS. 4A-4E show the impact of PsbS expression on photosynthesis and iWUE on plants grown under controlled conditions. In particular, FIG. 4A shows that there was no difference in net CO₂ assimilation (Aₙ) in plants with increased or decreased PsbS expression relative to WT plants. FIG. 4B shows that stomatal conductance levels were lower in plants with elevated PsbS expression relative to WT. Further, stomatal conductance levels were higher in plants with decreased PsbS expression relative to WT (FIG. 4B). Notably, the impact on stomatal conductance was larger than the impact on CO₂ assimilation (Aₙ), which was minimally affected.

FIG. 4C demonstrates that there was a strong positive correlation between iWUE and PsbS expression (R²=0.92, P=0.03, ANOVA). Thus, the data demonstrate that manipulation of PsbS expression significantly impacts iWUE. In particular, the data show that increased PsbS expression improves iWUE. FIG. 4D shows that the redox state of Q_{A} was significantly more oxidized in lines overexpressing PsbS compared to WT (R²=0.98, P <0.0001, ANOVA). FIG. 4E shows that there was a significant positive correlation between Q_{A} redox state and stomatal conductance (gₛ) in all tested plant lines. The results presented in FIGS. 4A-4E demonstrate that plants with elevated PsbS expression had improved iWUE when grown under controlled conditions. Further, the results presented in FIGS. 4A-4E show that there was a link between Q_{A} redox state and stomatal conductance.

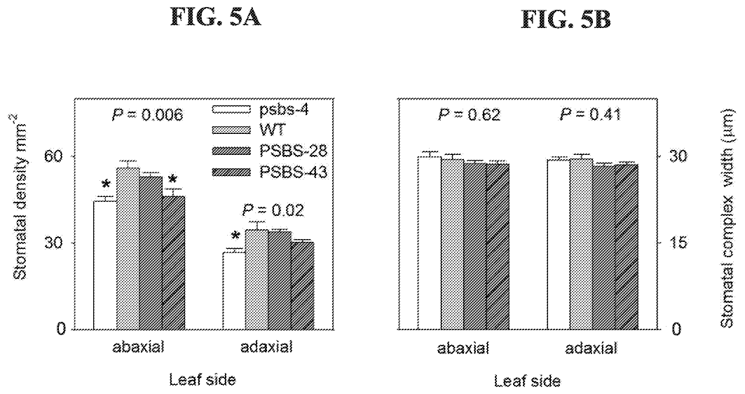

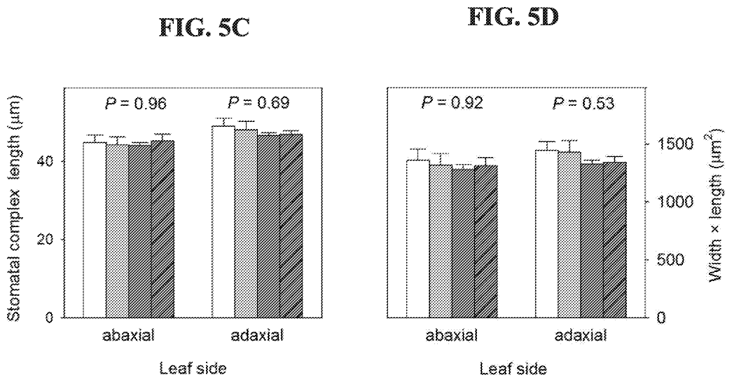

FIG. 5A shows that plants with elevated PsbS did not have increased stomatal density compared to WT plants. FIG. 5B shows that there was no difference in stomatal complex width in plants with altered PsbS expression compared to WT plants. FIG. 5C shows that there was no difference in stomatal complex length in plants with altered PsbS expression compared to WT plants. FIG. 5D shows that there was no difference in stomatal complex width×length (μM²) in plants with altered PsbS expression compared to WT plants. The results presented in FIGS. 5A-5D show that the change in stomatal conductance observed in plants with elevated PsbS expression was not due to any change in stomatal pore dimensions or stomatal density. Therefore, the combined data in FIGS. 4A-4E and 5A-5D demonstrate that the change in stomatal conductance linked to altered PsbS expression was due to regulation of stomatal opening.

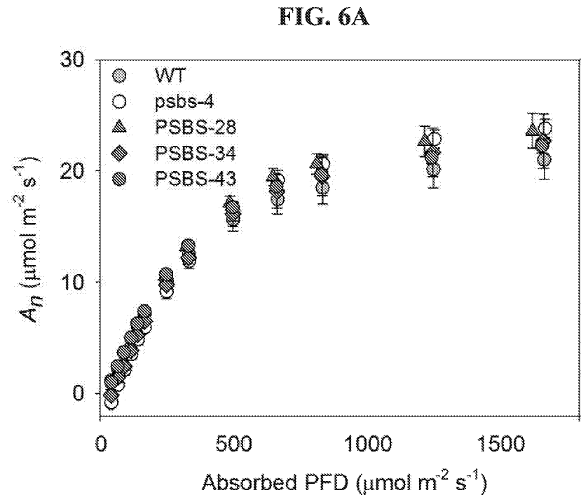

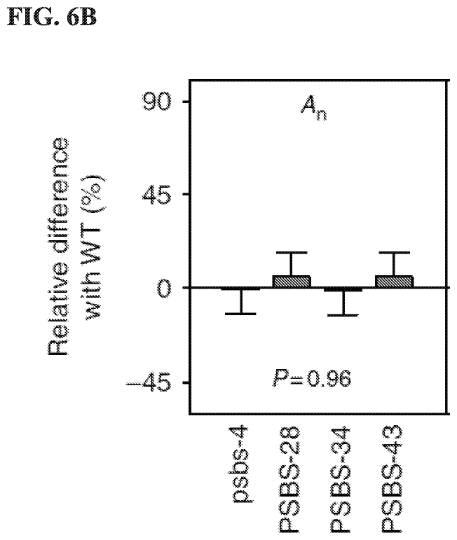

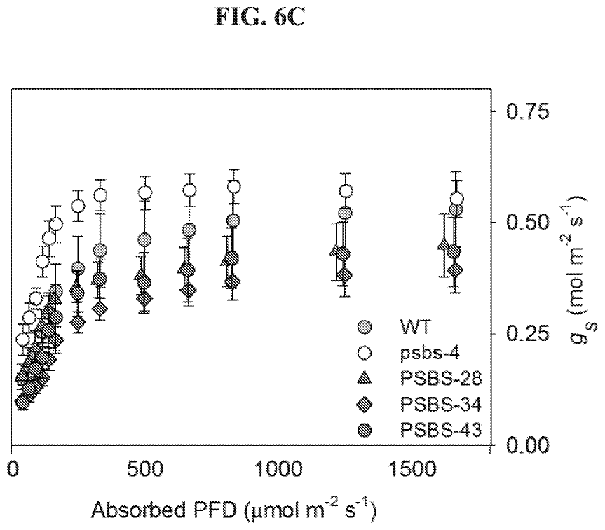

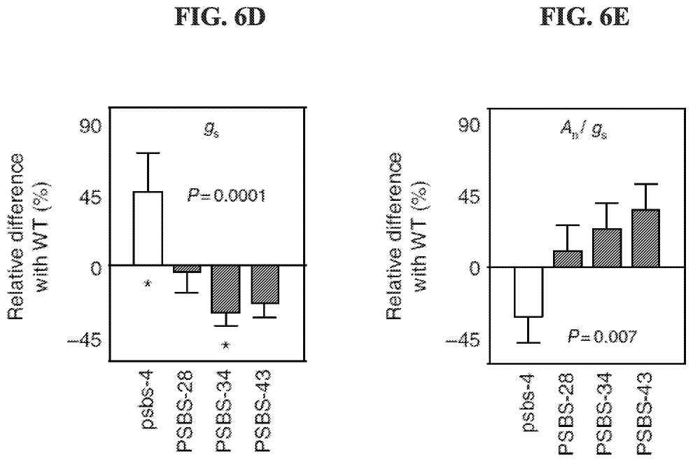

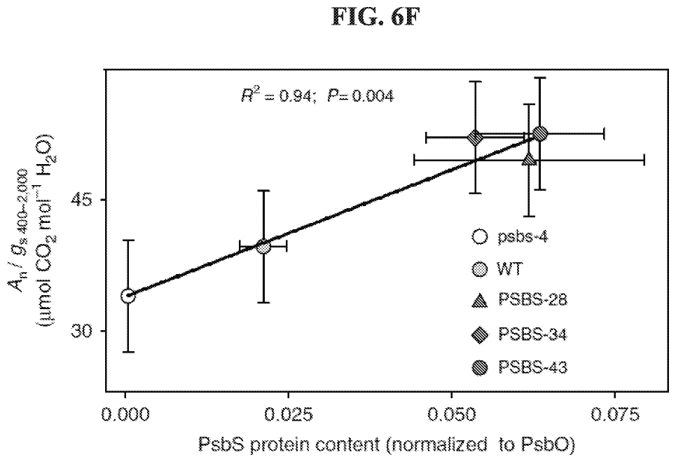

FIGS. 6A-6F show the impact of PsbS expression on photosynthesis and iWUE in plants under field-grown conditions. In particular, FIGS. 6A and 6B show that there was little difference in net CO₂ assimilation (Aₙ) in plants with increased or decreased PsbS expression. FIGS. 6C and 6D show that by contrast to A., stomatal conductance decreased in plants with elevated PsbS expression relative to WT. Further, plants with decreased PsbS expression had increased stomatal conductance relative to WT, and these effects were seen across all light levels, suggesting that increased iWUE occurred under all lighting conditions. (FIGS. 6C and 6D). FIG. 6E shows improved iWUE in N. tabacum plants with elevated PsbS expression relative to WT. However, the iWUE of N. tabacum plants with decreased PsbS expression dropped relative to WT (FIG. 6E). FIG. 6F demonstrates that there was a strong positive correlation and linear relationship between iWUE and PsbS expression (R²=0.94, P=0.004, ANOVA). The results presented in FIGS. 6A-6F demonstrate that plants with elevated PsbS expression had improved iWUE under field-grown conditions.

The results in this example demonstrate that plants with elevated PsbS expression had improved iWUE when grown in both controlled and field-grown conditions. The results further demonstrate that the change in stomatal conductance linked to altered PsbS expression was due to regulation of stomatal opening rather than changes in stomatal pore dimensions or stomatal density.

### Example 3

### PsbS Expression Does Not Affect Biochemical Photosynthetic Processes in Plants Grown Under Controlled Conditions

The following example describes the effect of altered PsbS expression on biochemical photosynthetic processes in plants grown under controlled conditions. These experiments were conducted to determine if potential alterations in biochemical photosynthetic processes following manipulation of PsbS expression (i.e., pleiotropic effects) could explain the change in iWUE.

### Materials and Methods

### Photosynthetic Gas Exchange Measurements

All photosynthetic gas exchange measurements were conducted as in Example 2.

### Rubisco Activation State and Content

The youngest fully expanded leaves were clamped in the cuvette of an open gas exchange system (LI6400XT with 2×3 LED Light Source, LI-COR) with light intensity set to 1,800 μmol m⁻² s⁻¹, CO₂ set to 400 μmol mol⁻¹, and block temperature set to 25° C. After steady-state gas exchange was reached, leaves were rapidly removed and a disc of 0.55 cm² from the center of the portion of the leaf that had been enclosed in the cuvette was snap frozen in liquid nitrogen. Rubisco activity was determined by the incorporation of ¹⁴CO₂ into acid-stable products at 25° C. Samples were ground in tenbroek glass homogenizers with ˜2 mL cm⁻² CO₂-free extraction buffer containing 100 mM Hepes-KOH (pH 7.5), 2 mM Na₂EDTA, 20 mM MgCl₂, 5 mM dithiothreitol (DTT), 5 mg mL⁻¹ polyvinyl pyrrolidine, 15 mM amino-n-caproic acid and 3.5 mM benzamidine, and 5% v/v protease inhibitor cocktail (Unless indicated otherwise all reagents from Sigma). Within 30 s of extraction, samples were assayed for initial Rubisco activity in a buffer containing 100 mM Bicine-NaOH (pH 8.2), 1 mM Na₂EDTA, 20 mM MgCl₂, 5 mM dithiothreitol (DTT), 1 mM ATP, 0.5 mM ribulose-1,5-bisphosphate, and 12.8 mM NaH¹⁴CO₃ (NaH¹⁴CO₃ purchased from Vitrax). Assays were run for 30 s and terminated with the addition of 300 μL 5 N formic acid. The radioactivity of acid-stable products was determined by liquid scintillation counting (Packard Tri-Carb 1900 TR, Canberra Packard Instruments Co.). After determining initial activity, the extract was incubated with 10 mM NaHCO₃ and 20 mM MgCl₂ for 20 min at room temperature, and the total activity of the extract was assayed as above. The activation state of Rubisco was determined by the ratio of initial activity to total activity.

### Results

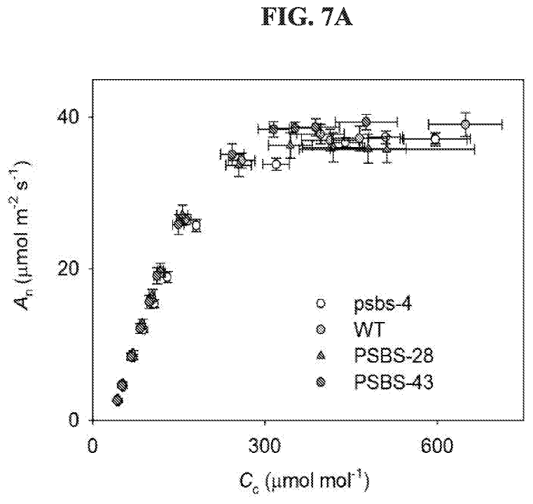

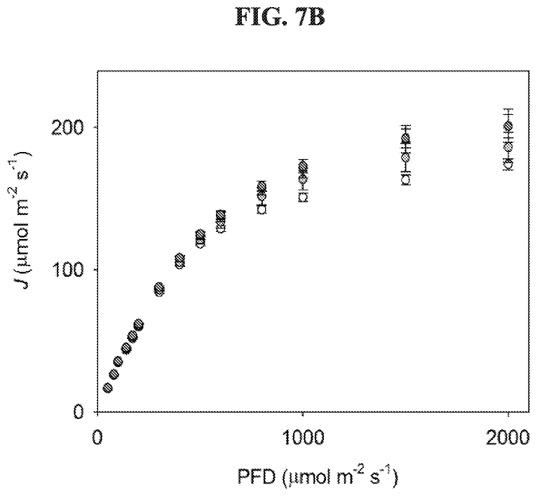

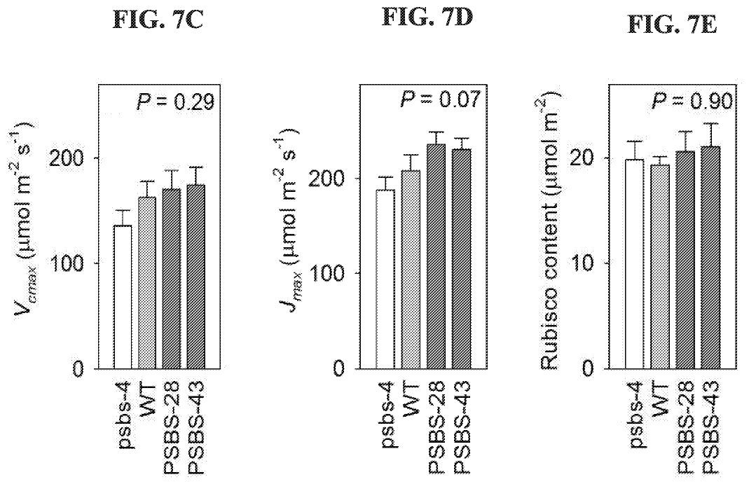

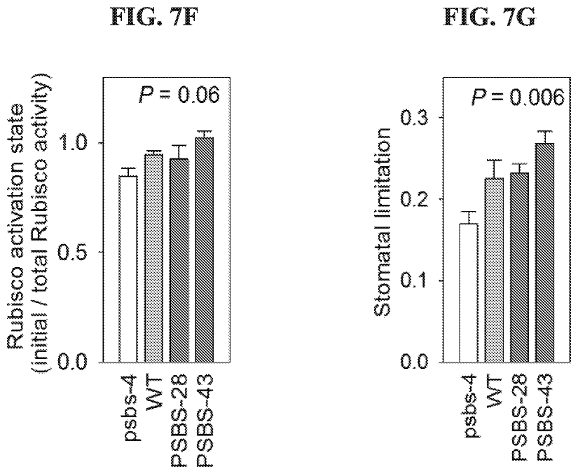

FIGS. 7A-7G show that the weak effects on photosynthetic capacity of PsbS overexpression lines balance the slight reduction in CO₂ availability due to greater stomatal limitation. In particular, PsbS overexpression lines had higher Jₘₐₓ (i.e., capacity of electron transport; FIG. 7D) and Rubisco activity (i.e., activation status of Rubisco; FIG. 7F). While these were both weak trends, they bordered on significance (Jₘₐₓ P=0.07 and Rubisco activation state P=0.06). FIG. 7E demonstrates that Rubisco content was similar between PsbS mutant lines and WT, indicating that the increased Rubisco activity was not merely due to increased Rubisco amount. Additional parameters were also used to measure photosynthetic activity. FIG. 7A shows that CO₂ fixation rate, as a function of chloroplastic CO₂ concentration, was slightly increased for PsbS overexpressing plants relative to WT. FIG. 7B demonstrates that electron transport rate, as a function of incident light, was slightly elevated for PsbS overexpressing plants relative to WT. FIG. 7C shows that maximum ribulose bisphosphate carboxylation capacity was slightly elevated in PsbS overexpressing plants relative to WT, whereas the maximum ribulose bisphosphate carboxylation capacity for plants with reduced PsbS expression was slightly reduced relative to WT. Finally, FIG. 7G shows that stomatal limitation rose with increased PsbS expression (i.e., there was a reduction in CO₂ availability), and fell with decreased PsbS expression (i.e., there was an increase in CO₂ availability). This effect was significant (P=0.006). These results, particularly those shown in FIG. 7D, FIG. 7F, and FIG. 7G, indicate that the weak effects on photosynthetic capacity (i.e., slight improvement of photosynthetic capacity) were able to balance the reduced CO₂ availability stemming from greater stomatal limitation (FIG. 7G) in the PsbS overexpression lines.

The results presented in Example 3 demonstrate that the PsbS overexpression lines were able to maintain photosynthetic rates while having lower stomatal conductance. Taken together, the results in Examples 2 and 3 show that the increase in iWUE observed following PsbS overexpression can be attributed to a decrease in stomatal conductance at all light levels, sufficient to decrease transpiration with minimal effect on CO₂ uptake. Thus, the decrease in stomatal conductance achieved through PsbS overexpression does not compromise photosynthesis.

### Example 4

### PsbS Expression Affects Biomass Productivity Traits in Plants Under Field-Grown Conditions

The following example describes the effect of altered PsbS expression on biomass productivity traits in plants under fully irrigated field-grown conditions.

### Materials and Methods

### Seedling Propagation for Field-Grown Conditions

Plants were grown as described above in Example 1.

### Biomass Productivity Trait Measurement

At final harvest, stem length and the number of leaves were determined, and leaf area was measured with a conveyor-belt scanner (LI-3100C Area meter, LI-COR). Leaf, stem, and root fractions were dried to constant weight at 60° C. after which the dry weights were determined.

### Results

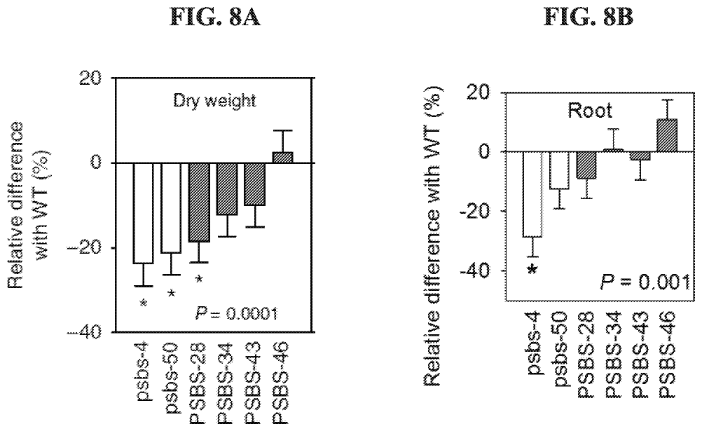

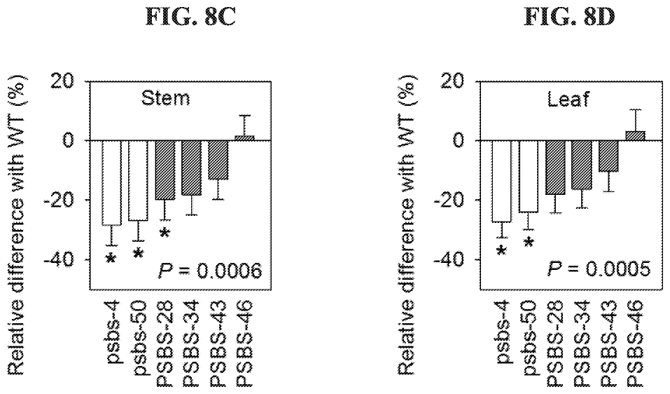

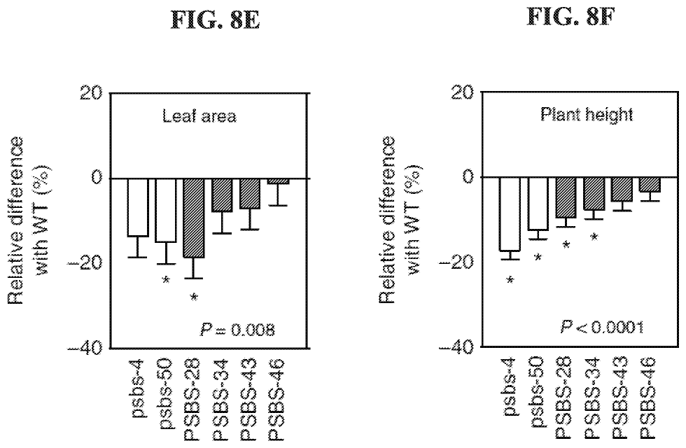

FIGS. 8A-8F show the impact of PsbS expression on biomass productivity for plants under field-grown conditions. FIGS. 8A-8D show total dry weight, root dry weight, stem dry weight, and leaf dry weight were significantly decreased in plants with reduced PsbS expression relative to WT. However, the data show that biomass productivity measures in plants with increased PsbS did not show a consistent response (FIGS. 8A-8F). It should be noted that growing the plants under fully-irrigated conditions (i.e., with sufficient water), was expected to offset the impact of improved iWUE on productivity, regardless of efficiency. Therefore, the inconsistent biomass productivity responses observed were maybe the result of the conditions under which these plants were grown. In addition, FIGS. 8E-8F show that leaf areas and plant height were significantly decreased in plants with reduced PsbS expression relative to WT. Plants with increased PsbS expression relative to WT also had decreased leaf area and plant height relative to WT. The results presented in FIGS. 8A-8F demonstrate that PsbS expression can alter biomass productivity traits in tobacco plants under field-grown conditions.

The results described in Examples 1-4 demonstrate that PsbS overexpression affected both iWUE and biomass productivity. Taken together, these results strongly indicate that manipulation of PsbS in crops may offer farmers an approach to improve iWUE and increase biomass productivity in water limited regions.

### Example 5

### PsbS Expression Affects Yield and/or Biomass Productivity Traits in Plants Under High Density Field-Grown Conditions Field-Grown Conditions

The following example describes experiments to investigate the effect of altered PsbS expression, using PsbS mutant lines psbs-4, psbs-50, PSBS-28, PSBS-34, PSBS-43, and PSBS-46, on biomass productivity under high density field-grown conditions.

### Materials and Methods

### Seedling Propagation for High Density Field-Grown Conditions

PsbS mutant lines psbs-4, psbs-50, PSBS-28, PSBS-34, PSBS-43, and PSBS-46 will be prepared and transferred as described in Example 1.

### High Density Field-Grown Conditions

Following initial propagation, seedlings will be transplanted to an experimental field site. As in Example 1, the field will be prepared 2 weeks prior to transplant by rototilling, cultivation, and harrowing. At this time, chlorpyrifos (1.5 g m⁻² Lorsban 15 G Insecticide, Dow AgroSciences) will be worked into the soil to suppress cutworm damage, sulfentrazone (29 μL m⁻² Spartan 4 F pre-emergence herbicide, FMC Agricultural Solutions) will be applied to reduce the emergence of weeds and slow-release fertilizer (30.8 g m⁻² ESN Smart Nitrogen, Agrium US Inc.) will be put down. After transplant, all seedlings will be sprayed with thiamethoxam (7 mg/plant Platinum 75 SG insecticide, Syngenta Crop Protection) to prevent damage from insect herbivory, and 12 days after the field transplant, all plants will be sprayed with fermentation solids, spores, and insecticidal toxins from Bacillus thuringiensis, (strain ABTS-351, DiPel Pro dry flowable biological insecticide, Valent Biosciences Corp.) to suppress tobacco hornworm. The field experiment will be set up as an incomplete randomized block design with 8 replicated plots of each of the 6 PsbS mutant lines and a WT control. 4 plots will consist of 6×6 plants spaced 30 cm apart. Further, 4 plots will consist of 12×12 plants spaced 15 cm apart. Each block will contain four rows of flour plants per genotype in north-south (N-S) orientation, surrounded by one border row of WT. The blocks will be positioned in 3 (N-S)×4 (E-W) rectangles with 75 cm spacing between blocks. The entire experiment will be surrounded by two border rows of WT plants. Standard irrigation will be applied during initial establishment, then withdrawn from all plots.

### Photosynthetic Gas Exchange Measurements

Photosynthetic gas exchange measurements will be conducted as described in Example 2.

### Rubisco Activation State and Content

Rubisco activation state and content measurements will be conducted as described in Example 3.

### Biomass Productivity Trait Measurement

Biomass productivity trait measurements will be conducted as described in Example 4.

### Soil Moisture

The vertical distribution of soil moisture (v/v) in each of the experimental plots (as described above in “High Density Field-Grown Conditions”) will be measured by lowering a capacitance sensor through an access tube in the middle of each plot (Diviner-2000, Sentek Technologies, Adelaide, Australia). Measurements will be made at 2 day intervals and coupled with precipitation measurement will provide a measure of water use in each plot (Gray et al. 2016, Nature Plants 16132). Raw data obtained from the capacitance probe will be calibrated against gravimetric data as described by previously (Paltineanu et al. 1997 Soil Sci. Soc. Am. J. 61, 1576).

### Plant Water Potential

To measure the leaf water potential, samples will be collected from three plants from each plot at two day intervals. Samples will be collected at midday from the field, corresponding to midday gas exchange measurements. Five leaf discs of approximately 1.2 cm in diameter will be collected from each plant and immediately sealed in psychrometer chambers (C-30; Wescor Environmental Products, Logan, Utah). As previously described, samples will be equilibrated at 25° C., and an integrated dew-point micro-voltometer (HR-33T; Wescor) will be used to measure water potential in each psychrometer chamber (Leakey et al. 2006, Plant Cell Environ. 29, 1794).

### Results

Final yield is measured, and shows that those lines overexpressing PsbS have higher yields, especially at the higher planting density. Yet, the water use of lines overexpressing PsbS is no greater, showing that the plants over-expressing PsbS yield more for the same amount, or a lesser amount, of water than the controls. Further, at mid-day, leaf water potential of the plants overexpressing PsbS equals, or is higher, than that of the controls, showing that these plants are no more, or less, stressed than the controls despite higher productivity.

### Example 6

### PsbS Overexpression in Glycine Max (Soybean) Plants Under Controlled and Field-Grown Conditions

The following example describes the development of Glycine max (G. max) lines with altered PsbS expression, and the assessment of these lines under controlled and field-grown conditions.

### Materials and Methods

Developing G. max Lines with Altered PsbS Expression

The pEG100-NbPsbS (FIG. 9) construct described in Example 1 will be codon-optimized for soybean, and used to develop G. max lines with altered PsbS expression. Soybean-optimized pEG100-NbPsbS will be transformed into an elite G. max cultivar using the Agrobacterium tumefaciens-mediated protocol (Olhoft, P. M. et al., Soybean (Glycine max) Transformation Using Mature Cotyledonary Node Explants, and Ko, T.-S. et al., Soybean (Glycine max) Transformation Using Immature Cotyledon Explants, in Agrobacterium Protocols, K. Wang, Editor. 2006, Humana Press Inc: Totowa, NJ. p. 385-406) with the aim of obtaining single copy transformants of the elite G. max cultivar. Then, copy number and homozygosity will be assessed using digital droplet PCR (Glowacka, K., et al., An evaluation of new and established methods to determine T-DNA copy number and homozygosity in transgenic plants. Plant Cell and Environment, 2016. 39: p. 908-917).

A second construct will also be made, in which the dicot rbcS promoter is used in place of the 35S promoter. This construct will be used to develop G. max lines with altered PsbS expression as described above.

Sixty independent transformants will be produced for each construct. Modulated chlorophyll fluorescence imaging will be used to detect the transformants showing the largest increase in the amplitude of NPQ as a means to identifying 10 lines expressing PsbS most strongly. NPQ measurements to identify lines showing the largest increase in the amplitude of NPQ will be performed as described in Example 1. These 10 lines will then be screened for expression of the transgene mRNA and recombinant protein, and copy number. mRNA expression analysis and protein expression analysis will be performed as described in Example 1.

Soybean is a seed-planted crop, and so single copy transformants will be selected to obtain seed homozygous for the transgene. Seedling propagation for controlled condition experiments will be performed as described in Example 1. These will then be screened for improvement of instantaneous water use efficiency (iWUE) under controlled conditions. Screening for improved iWUE will be performed as described in Example 2. The lines showing the highest iWUE will be screened for increased whole plant water use efficiency (WUE) under greenhouse or artificial controlled environment conditions using load cells to simultaneously track plant mass accumulation and mass loss of water (Negin, B. and M. Moshelion, The advantages of functional phenotyping in pre-field screening for drought-tolerant crops. Functional Plant Biology, 2017. 44(1): p. 107-118).

The three independent transformants showing high WUE without compromise to production will then be multiplied up for field trials. Soybean will be planted following established agronomic recommendations for maximizing the yield of soybean. Field-grown conditions will be conducted as in Example 1 at a sites climatically suited to G. max (e.g., Illinois). 8 plots of 4 rows of each independent transformant will be planted. 4 plots will be irrigated to field capacity and 4 plots rainfed only. An access tube for time domain reflectometry (TDR) will be inserted in the center of each plot prior to planting, following procedures previously used for soybean field trials (Gray, S. B., et al., Intensifying drought eliminates the expected benefits of elevated carbon dioxide for soybean. Nature Plants, 2016. 2(9)). An onsite meteorological station, along with TDR, will be used to determine daily plot water use. Growth will be monitored over the course of the season and final productivity will be determined by harvest at crop dry down as in Example 4.

Trials will then be undertaken at up to 5 locations within the typical growing region(s) of the given crop (e.g., Illinois), to establish the efficacy of the trait in a wider range of environments.

### Example 7

### PsbS Overexpression in Zea Mays (Maize) Plants Under Controlled and Field-Grown Conditions

The following example describes the development of Zea mays (Z. mays) lines with altered PsbS expression, and the assessment of these lines under controlled and field-grown conditions.

### Materials and Methods

The constructs used to develop Z. mays lines with altered PsbS expression will contain the Sorghum bicolor SbPsbs gene coding sequence (e.g., encoding the protein of SEQ ID NO: 154) cloned between the mesophyll plastid specific C4-Pepc promoter and octopine synthase terminator in a plasmid vector. The vector will be transformed into Z. mays inbred line B73 using the Agrobacterium tumefaciens-mediated protocol. The further development of Z. mays lines with altered PsbS expression will proceed as described in Example 6, with the exception that the transformed inbred line will be crossed with a second productive inbred line to provide hybrid seed, because in maize the commercial seed is hybrid. The lines will be planted following established agronomic recommendations for maximizing the yield of maize, and tested at a site climatically suited to maize (e.g., Illinois). Otherwise, all experimental protocols and analyses will be performed as in Example 6.

### Example 8

### PsbS Overexpression in Triticum aestivum (Wheat) or Oryza sativa (Rice) Plants Under Controlled and Field-Grown Conditions

The following example describes the development of Triticum aestivum (T. aestivum) or Oryza sativa (O. sativa) lines with altered PsbS expression, and the assessment of these lines under controlled and field-grown conditions.

### Materials and Methods

The constructs used to develop T. aestivum or O. sativa lines with altered PsbS expression will contain the Brachypodium distachyon BdPsbs gene coding sequence (e.g., encoding the protein of SEQ ID NO: 160) cloned between either the 35S promoter or the rbcs promoter and the octopine synthase terminator in a plasmid vector. The further development of T. aestivum or O. sativa lines with altered PsbS expression will proceed as described in Example 6, and all experimental protocols and analyses will be performed as in Example 6.

### Example 9

### PsbS Overexpression in Vigna unguiculata (Cowpea), Lycopersicon esculentum (Tomato), Solanum melogena (Euplant), Solanum tuberosum (Potato), Manihot esculenta (Cassava), or Gossypium hirsutum (Cotton) Plants Under Controlled and Field-Grown Conditions

The following example describes the development and validation of Vigna unguiculata (V. unguiculata), Lycopersicon esculentum (L. esculentum), Solanum melogena (S. melogena), Solanum tuberosum (S. tuberosum), Manihot esculenta (M. esculenta), or Gossypium hirsutum (G. hirsutum) lines with altered PsbS expression grown under controlled and field-grown conditions.

### Materials and Methods

The constructs used to develop V. unguiculata, L. esculentum, S. melogena, S. tuberosum, M. esculenta, or G. hirsutum lines with altered PsbS expression will be codon-optimized for each crop. Each crop will be planted following established agronomic recommendations for maximizing the yield of the given crop, and tested at a site climatically suited to the crop (e.g., Puerto Rico for cowpea and cassava). All experimental protocols and analyses will be performed as in Example 6.

What is claimed is:

1. A genetically altered plant or part thereof comprising one or more genetic alterations that increase activity of a PsbS protein as compared to a WT plant without the one or more genetic alterations, wherein the genetically altered plant shows increased water use efficiency as compared to the WT plant grown under the same conditions, and wherein the genetically altered plant or a progenitor thereof was selected for increased water use efficiency compared to the WT plant grown under the same conditions.

2. The genetically altered plant of claim 1, wherein the conditions are lower irrigation conditions than standard irrigation conditions for the WT plant, rain fed conditions, higher density growth conditions than standard growth density conditions for the WT plant, higher salinity conditions than standard salinity conditions for the WT plant, or conditions resulting in wet leaf surfaces.

3. The genetically altered plant of claim 2, wherein the increased activity of the PsbS protein provides the genetically altered plant with a higher yield, an increased growth rate, an increased tolerance of salinity, an increased ability to withstand salinity, an increased flow of nutrients to the roots, an increased availability of nutrients over time, an increased utilization of fertilizer, an increased utilization of nutrients, a decreased susceptibility to a plant disease requiring wet leaf surfaces for infection, a decreased susceptibility to infection by the plant disease, a reduced incidence of the plant disease, or a reduced incidence of infection by the plant disease, in each case as compared to the WT plant without the increased activity grown under the same conditions.

4. The genetically altered plant of claim 1, wherein the genetically altered plant does not comprise increased activity of zeaxanthin epoxidase (ZEP) protein, violaxanthin de-epoxidase (VDE) protein, or both, as compared to a WT plant, and wherein the genetically altered plant does not comprise reduced activity of K+ efflux antiporter 3 (KEA3) as compared to a WT plant.

5. The genetically altered plant of claim 1, wherein the increased activity of the PsbS protein provides the genetically altered plant with substantially similar photosynthetic efficiency as compared to the WT plant grown under the same conditions where the activity of the PsbS protein is increased.

6. The genetically altered plant of claim 2, wherein increased activity is increased expression.

7. The genetically altered plant of claim 6, wherein the increased expression is due to expression of a heterologous PsbS protein.

8. The genetically altered plant of claim 7, wherein the heterologous PsbS protein is encoded by a first nucleic acid and the first nucleic acid is operably linked to a second nucleic acid comprising a promoter, and wherein the first nucleic acid sequence and the second nucleic acid sequence are stably integrated into a nuclear genome of the plant or into a chloroplast genome of the plant.

9. The genetically altered plant of claim 8, wherein the promoter is selected from the group of a CaMV35S promoter, a ubiquitin promoter, a Rbcs1A promoter, a GAPA-1 promoter, a FBA2 promoter, or any combination thereof

10. The genetically altered plant of claim 8, wherein the heterologous PsbS protein comprises a protein with at least 70% sequence identity to, at least 75% sequence identity to, at least 80% sequence identity to, at least 85% sequence identity to, at least 90% sequence identity to, at least 95% sequence identity to, or at least 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

11. The genetically altered plant of claim 8, wherein the heterologous PsbS protein comprises a glutamate at a position corresponding to amino acid 149 of reference sequence SEQ ID NO: 21 and a glutamate at a position corresponding to amino acid 255 of reference sequence SEQ ID No: 21.

12. The genetically altered plant of claim 6, wherein the increased expression is due to overexpression of an endogenous PsbS protein.

13. The genetically altered plant of claim 12, wherein overexpression of the endogenous PsbS protein was achieved using a gene editing technique to introduce the one or more genetic alterations that increase the activity of the endogenous PsbS protein, and wherein the gene editing technique is a transcription activator-like effector nuclease (TALEN) gene editing technique, a clustered Regularly Interspaced Short Palindromic Repeat (CRISPR/Cas) gene editing technique, a zinc-finger nuclease (ZFN) gene editing technique, or combinations of the foregoing.

14. The genetically altered plant of claim 1, wherein the PsbS protein is localized to a thylakoid membrane of at least one chloroplast within a chloroplast containing leaf cell of the genetically altered plant, optionally wherein the cell is a mesophyll cell or a guard cell.

15. A plant part of the genetically altered plant of claim 1, wherein the plant part comprises a leaf, a stem, a root, a flower, a seed, a kernel, a grain, a pod, a bean, a pea, a fruit, a chloroplast, a cell, or a portion thereof, and wherein the genetically altered plant part includes the one or more genetic alterations.

16. A genetically altered tissue culture produced from protoplasts or cells from the genetically altered plant of claim 1, wherein the cells or protoplasts are produced from a plant part selected from the group of leaf, leaf mesophyll cell, anther, pistil, stem, petiole, root, root tip, fruit, seed, kernel, grain, pod, bean, pea, flower, cotyledon, hypocotyl, embryo, or meristematic cell, wherein genetically altered tissue culture includes the one or more genetic alterations.

17. A genetically altered plant regenerated from the genetically altered tissue culture of claim 16, wherein the genetically altered plant includes the one or more genetic alterations.

18. The genetically altered plant of claim 1, wherein the plant is not a tobacco plant.

19. A genetically altered plant or part thereof comprising one or more genetic alterations that increase activity of a PsbS protein as compared to a WT plant without the one or more genetic alterations, wherein the genetically altered plant shows increased water use efficiency as compared to the WT plant grown under the same conditions.

20. The genetically altered plant of claim 19, wherein the genetically altered plant does not comprise increased activity of zeaxanthin epoxidase (ZEP) protein, violaxanthin de-epoxidase (VDE) protein, or both, as compared to a WT plant, and wherein the genetically altered plant does not comprise reduced activity of K+ efflux antiporter 3 (KEA3) as compared to a WT plant.
